SEQUENCE LISTING (26 sequences)

SEQ ID NO: 1 - protein, 9 aa, synthetic construct
  REGION 1..9: note = Synthetic
GYSITSGYY

SEQ ID NO: 2 - protein, 7 aa, synthetic construct
  REGION 1..7: note = Synthetic
ITFDGAN

SEQ ID NO: 3 - protein, 12 aa, synthetic construct
  REGION 1..12: note = Synthetic
TRSSYDYDVLDY

SEQ ID NO: 4 - protein, 6 aa, synthetic construct
  REGION 1..6: note = Synthetic
QDISNF

SEQ ID NO: 5 - skipped

SEQ ID NO: 6 - protein, 9 aa, synthetic construct
  REGION 1..9: note = Synthetic
QQGHTLPYT

SEQ ID NO: 7 - protein, 6 aa, synthetic construct
  REGION 1..6: note = Synthetic
SGYYWN

SEQ ID NO: 8 - protein, 16 aa, synthetic construct
  REGION 1..16: note = Synthetic
YITFDGANNYNPSLKN

SEQ ID NO: 9 - protein, 10 aa, synthetic construct
  REGION 1..10: note = Synthetic
SSYDYDVLDY

SEQ ID NO: 10 - protein, 11 aa, synthetic construct
  REGION 1..11: note = Synthetic
RASQDISNFLN

SEQ ID NO: 11 - protein, 7 aa, synthetic construct
  REGION 1..7: note = Synthetic
YTSRLHS

SEQ ID NO: 12 - protein, 8 aa, synthetic construct
  REGION 1..8: note = Synthetic
GYSITSGY

SEQ ID NO: 13 - skipped

SEQ ID NO: 14 - protein, 8 aa, synthetic construct
  REGION 1..8: note = Synthetic
SYDYDVLD

SEQ ID NO: 15 - protein, 7 aa, synthetic construct
  REGION 1..7: note = Synthetic
SQDISNF

SEQ ID NO: 16 - protein, 6 aa, synthetic construct
  REGION 1..6: note = Synthetic
GHTLPY

SEQ ID NO: 17 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - RNA, 16 nt, synthetic construct
  misc_feature 1..16: note = Synthetic
  source 1..16: mol_type = other RNA
cagcgcccaccagtca

SEQ ID NO: 22 - DNA, 16 bp, synthetic construct
  misc_feature 1..16: note = Synthetic
  source 1..16: mol_type = other DNA
tgactggtgggcgctg

SEQ ID NO: 23 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 24 - DNA, 2859 bp, Homo sapiens
  source 1..2859: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctgga

SEQ ID NO: 25 - DNA, 2683 bp, Mus musculus
  source 1..2683: mol_type = genomic DNA
gaactggccagagagacccaagggatagtcagggacgggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcgccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaa

SEQ ID NO: 26 - DNA, 19841 bp, Homo sapiens
  source 1..19841: mol_type = genomic DNA
tttcctacttgaagcctgacgtagtaaagatcgggggagggttagacagatacagtggtccccaaccattttggcaccagggactggtcttatggaagacagtttttccacagactgttggggggatggttttgggatgaaaccgttctgcctctgatcatcaggtgttagattctaataaggagcgcacacctagatccctcgcatgcatagttcatggtggggttcgcactcttacgaggattgaatggtgcgctgctccggtaggaggctgggctcaggctgtaatgcctgctcgcccaccactcacctcctgctgcatggcctggttcctaacaggccacggaccactactgttccatggcccggaggttgaggaccccgagatacaggacaattctgtggcaagcaggactgtcccctcgccaaagatgggacatcgaggctccttggagcaccctgtggccaccttgcagcagcctctgtttccccatgtttccatgacctggtgtccatctgtcttccccagtttgggagcttctctccgaggaggacctgggcctggtgtggcacctgctgtgtgagcggggccatgtccaacggccttcttggggactttgggtcggggagaagttctgcctgggttttactgccttctcccaaccccacactgtctcccctggcagcggttgatcgacaagaccaaggtgacatatctgaagtggctgcctgagtcggagagcctgttcctggcatcacacgccagtggccacctgtacctgtacaacgtcagccacccctgcgcctcggccccgccccagtacagcctgctgaagcagggcgagggcttctctgtctatgctgccaagagcaaggcaccccgcaacccgctggccaagtgggcggtgggtgaggggcccctcaacgagttcgccttctcgcccgatggccggcacctggcctgtgtgagccaggatggctgcctgcgcgtcttccacttcgactccatgctcctgcgtgggctcatgaagagctactttgggggcctgctgtgtgtgtgctggagccctgacggccgctacgtggtgacgggtggcgaagatgacctggtcaccgtgtggtccttcaccgagggccgcgtggtggctcgaggccatggccacaagtcctgggtcaacgctgtggcctttgacccctacaccacaagggcagaggaggcggcgacagcagccggtgctgatggggagcggagcggcgaagaggaggaggaggagcccgaggctgcgggcacaggctcggccgggggcgccccgctctctccactgcccaaggctggctccattacttaccgctttggctcggcgggccaggacacgcagttctgcctgtgggacctcactgaagacgtgctctacccgcacccccccctggcccgcacccgcaccctccctggcacacctggcaccacgccaccggccgccagcagctcgaggggtggcgagcctggcccaggccccctgcctcgctcgctgtcccgctccaacagtctcccgcacccagctggcgggggcaaggcgggcggcccgggtgtggcggcagagcctggcacaccattcagcattggccgcttcgccacgctcacactgcaggagcggcgggaccggggggcagagaaggagcacaagcgctaccacagcctgggcaacatcagccggggtggcagtggcggcagtggcagtggtggggagaagcccagcggccctgttccccgcagccgcctggaccccgccaaggtgctgggcactgcgctgtgcccgcgcatccacgaggtgcccctgctggagccccttgtgtgcaagaagatcgcccaggagcggctcacagtcctcctgttcctggaggactgcatcatcactgcctgccaggagggcctcatctgcacctgggcccggccgggcaaggcggtgagtggccccacaccagcctgccggggacctggcaggacctttcgtgggaagaggcaggcattggcagagagagggctttgttgctgtcacagcctctggctccgtggggtgaggggaagccagggaaatcttagtgtctcagtacaagacctctcagatccttagagtgagggggtctagccctaggcagcaggcagcagaaagaggggtgggtgtgagagccagctaggaattggggcatccaaggctggccgtctgaagggcagcagatgggccccacatggccaggtcttactgcctgtcactcgaaccagaatctatttctgttgaacatctgttttttaaatcgtgaaacttttttgagtacttcaggccaaaactaggggcgagctcaagcctgtgggcatggctgccagcctgggtctgggactcaggatctgagcctcctgctgaaggcacaggctgggaatcccaggcctgggttccagtcccactccctctgtgaccctggacaagtcactgccccctctgacctccaactcatcacctcttagaacagagcctgtaggatgggcagtgggtggatgtgcttgcctcctgggtgggctgtggcgttgggaaggtcatagtaggcgaatcaggcctggcatcttgtaagttcggagctcgtcttgggtgtctcagcttcttagggcttggactcagttgcccagggtcctggaggccgtggcttggttcctcagatcctcagttttggaatcgtagagtcctgagtccctagaacttgagagcacagtctgagtgactcagaggcaagagtggtgggatttggggagtctggttgagtcctaaaagagacccctctgtctccgtagttcacagacgaggagaccgaggcccagacaggggaaggaagttggcccaggtcacccagcaagtcagtggtagaggtaggactgtccctgagttctttccccagcacctcagggtccctcccaagttagaagggagctccagtttccccctcccctcccacccttacccttaccccatggtctcactcaggatccgccaaggactttgattattgcgtgaaagtgctgactgccaggacaggaagctagctaagatgcaagttcccagcctagagcagtggcctctggggggtctagggcggacccaagggcaaggccagggtggcagcagctttggggactctgggctggctccctccccttgacactggctgaagcccaggtggtctctaacccctcccatctctccctctcatcttccccagggcatctcctcccaaccaggcaactccccgagtggcacagtggtgtgaagccatggatatcgggcccccccaaccccatgcccccagcctcctagccataaccctccctgctgacctcacagatcaacgtattaacaagactaaccatgatggatggactgctccagtccccccacctgcacaaaatttgggggccccccagactggcccggacacgggcgatgtaatagcccttgtggcctcagccttgtcccccacccactgccaagtacaatgacctcttcctctgaaacatcagtgttaccctcatccctgtccccagcatgtgactggtcactcctggggagagactccccgcccctgccacaagagccccaggtctgcagtgtgcccctcagttgagtgggcagggccgggggtggtccagccctcgcccggcccccaccccagctgcccttgctattgtctgtgcttttgaagagtgttaaattatggaagcccctcaggttcctccctgtcccgcaggacctcttatttatactaaagttccctgttttctcagcgggtctgtccccttcggaggagatgatgtagaggacctgtgtgtgtactctgtggttctaggcagtccgctttccccagaggaggagtgcaggcctgctcccagcccagcgcctcccaccccttttcatagcaggaaaagccggagcccagggagggaacggacctgcgagtcacacaactggtgacccacaccagcggctggagcaggaccctcttggggagaagagcatcctgcccgcagccagggcccctcatcaaagtcctcggtgttttttaaattatcagaactgcccaggaccacgtttcccaggccctgcccagctgggactcctcggtccttgcctcctagtttctcaggcctggccctctcaaggcccaggcaccccaggccggttggaggccccgacttccactctggagaaccgtccaccctggaaagaagagctcagattcctcttggctctcggagccgcagggagtgtgtcttcccgcgccaccctccaccccccgaaatgtttctgtttctaatcccagcctgggcaggaatgtggctccccggccaggggccaaggagctattttggggtctcgtttgcccagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaaagctggtataaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgtttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtcctcctggacggcccgttaccttctgcgttgtcccgatggggaaactgaggccctgagccagaagcacacgctggggggaggcagaaagcgcggccagaggcggagggaaaacaaagggagaatcacagacagacgggagggggacggacacacacaaggggacagagacccgagtggagagctggatctcgccttcccggcgtggggcgcagggtcggccagaaagaagatcgagaagagcggggagtgggggcgaaaaggggggacaggtgggggaggaggctggggaaagcccgagggaggaagagagggagggaggaacttcccaaagttgcaaaacatggctaccttgcctgcggagccgagcgcggggccggcggctgggggggaggcggtggcggcggcggcggcgaccgaagaggaggaggaggaagcgcgccagctcttgcagactttgcaggcggccgagggtgaggcggcggcggcggccggggccggggcgggcgcagcggctgcgggagctgagggcccgggatccccgggcgtccccgggtcgccccccgaggccgcttccgaaccgcccacgggcctccgcttctcgcccgagcaggtggcgtgcgtctgcgaggcgctgctccaggcgggccacgccggccgcttgagccgcttcctgggcgcactgcccccggccgagcgcctacgtggcagcgacccggtgttgcgcgcgcgggccctggtggccttccagcggggcgagtacgccgagctctaccggctactcgagagccgccccttccccgccgcccaccacgccttcctgcaggacctctacctgcgcgcgcgctaccatgaggccgagcgggcccgcggccgcgcgcttggcgcagtggacaagtatcgactgcgcaagaagttcccgctgcccaagaccatctgggacggcgaggagacagtctactgcttcaaggagcgctcccgcgcagcgctcaaggcctgctaccgcggcaaccgctaccccacgccggacgagaagcgccgcctggccacactcaccggcctgtcgctcacgcaggtcagcaactggttcaagaaccggcgacagcgcgaccggaccggggccggaggcggcgcgccctgcaagaggtgaggggcctcgggcggcgcaagtccagctctcccggggacatcccgtccaccagccctcttcccccgtgcccactgctggggccggcgcgccgaggtcctcggacatctcccgggaccagctcacaatctcaggcgcccgcggggcgcggggactaagtgtggacgggacaggcacccgcccgggccctctccccgcacgcgtctcctcttccagcggctccattccgagctccttcccaaatcccatcggtgttggggaatcacactgcggggggcactagagggactgaggaaaaaggacagggcctgtggccactccact

(19) United States
(12) Patent Application Publication
(10) Pub. No.: US 2025/0249117 A1
(43) Pub. Date: Aug. 7, 2025

(54) DOSING OF MUSCLE TARGETING COMPLEXES FOR TREATING MYOTONIC DYSTROPHY

(71) Applicant: Dyne Therapeutics, Inc., Waltham, MA (US)

(72) Inventors: Stefano Zanotti, Waltham, MA (US); Tyler Picariello, Waltham, MA (US); Oxana Beskrovnaya, Waltham, MA (US); John Davis, Waltham, MA (US); Tracy McGregor, Boxborough, MA (US); Clarence Edwin Garner, Placitas, NM (US); Curtis Johnston, Tariffville, CT (US); Timothy Weeden, Waltham, MA (US); John Najim, Waltham, MA (US); Romesh R. Subramanian, Framingham, MA (US); Mohammed T. Qatanani, Waltham, MA (US); Cody A. Desjardins, Waltham, MA (US); Kim Tang, Belmont, MA (US); Brendan Quinn, Waltham, MA (US); Scott Hilderbrand, Waltham, MA (US)

(73) Assignee: Dyne Therapeutics, Inc., Waltham, MA (US)

(21) Appl. No.: 18/856,266

(22) PCT Filed: Apr. 14, 2023
(86) PCT No.: PCT/US2023/065774

§ 371 Date: Oct. 11, 2024

Related U.S. Application Data
(60) Provisional application No. 63/368,691, filed on Jul. 18, 2022.
(60) Provisional application No. 63/331,733, filed on Apr. 15, 2022.

(51) Int. Cl. A61K 47/68; C07K 16/28; C12N 15/113

(52) U.S. Cl. CPC A61K 47/6807; A61K 47/6849; C07K 16/2881; C12N 15/1137; C12Y 207/11001; C07K 2317/55; C12N 2310/11; C12N 2310/313; C12N 2310/321; C12N 2310/3231

(57) ABSTRACT

Aspects of the disclosure relate to methods of reducing expression or activity of DMPK (e.g., reducing the level of a mutant or wild-type DMPK RNA, or the activity of a DMPK gene product) and/or methods of treating myotonic dystrophy (e.g., DM1) in a subject. In some embodiments, the methods comprise administering to the subject a composition comprising complexes (e.g., muscle targeting complexes) comprising an oligonucleotide (e.g., a DMPK—targeting oligonucleotide) covalently linked to an antibody (e.g., anti-TfR1 antibody).

### RELATED APPLICATIONS

This application claims the benefit under 35 U.S.C. § 119(e) of U.S. Provisional Application Ser. No. 63/368,691, entitled “DOSING OF MUSCLE TARGETING COMPLEXES FOR TREATING MYOTONIC DYSTROPHY,” filed on Jul. 18, 2022, and of U.S. Provisional Application Ser. No. 63/331,733, entitled “DOSING OF MUSCLE TARGETING COMPLEXES FOR TREATING MYOTONIC DYSTROPHY,” filed on Apr. 15, 2022, the entire contents of each of which are incorporated herein by reference.

### FIELD OF THE INVENTION

The present application relates to targeting complexes for delivering an effective amount of oligonucleotide molecular payloads to cells and uses thereof, particularly uses relating to treatment of disease.

### REFERENCE TO AN ELECTRONIC SEQUENCE LISTING

The contents of the electronic sequence listing (D082470077W000-SEQ-COB.xml; Size: 56,306 bytes; and Date of Creation: Apr. 7, 2023) are herein incorporated by reference in their entirety.

### BACKGROUND

Myotonic dystrophy (DM) is a dominantly inherited genetic disease that is characterized by myotonia, muscle loss or degeneration, diminished muscle function, insulin resistance, cardiac arrhythmia, smooth muscle dysfunction, and neurological abnormalities. DM is the most common form of adult-onset muscular dystrophy, with a worldwide incidence of about 1 in 8000 people worldwide. Two types of the disease, myotonic dystrophy type 1 (DM1) and myotonic dystrophy type 2 (DM2), have been described. DM1, the more common form of the disease, results from a repeat expansion of a CTG trinucleotide repeat in the 3′ non-coding region of DMPK on chromosome 19; DM2 results from a repeat expansion of a CCTG tetranucleotide repeat in the first intron of ZNF9 on chromosome 3. In DM1 patients, the repeat expansion of a CTG trinucleotide repeat, which may comprise greater than about 50 to about 3,000 or more total repeats, leads to generation of toxic RNA repeats capable of forming hairpin structures that bind essential intracellular proteins, e.g. muscleblind-like proteins, with high affinity resulting in protein sequestration and the loss-of-function phenotypes that are characteristic of the disease. Apart from supportive care and treatments to address the symptoms of the disease, no effective therapeutic for DM1 is currently available.

### SUMMARY

According to some aspects, the present disclosure provides methods (e.g., methods of delivering oligonucleotides to a subject, methods of administering complexes to a subject, methods of reducing expression or activity of DMPK in a subject, and/or methods of treating myotonic dystrophy (e.g., DM1) in a subject), the method comprising administering to the subject a composition comprising an effective amount of complexes comprising an anti-transferrin receptor 1 (TfR1) antibody covalently linked to one or more oligonucleotides.

According to some aspects, a method of reducing DMPK expression in a subject provided herein comprises administering to the subject a composition comprising an effective amount of complexes comprising an anti-transferrin receptor 1 (TfR1) antibody covalently linked to one or more oligonucleotides, wherein the effective amount provides to the subject 5 mg to 110 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotides of the complexes comprise the nucleobase sequence of CAGCGCCCACCAGUCA (SEQ ID NO: 21).

In some embodiments, the oligonucleotides of the complexes comprise a 5′-X—Y—Z-3′ configuration, wherein X and Z are flanking regions comprising one or more modified nucleosides and Y is a gap region comprising one or more 2′-deoxyribonucleosides.

In some embodiments, each complex comprises a structure of formula (I): [R¹]ₙ₁—R², wherein:
each R¹ comprises a group of the formula (Ia):

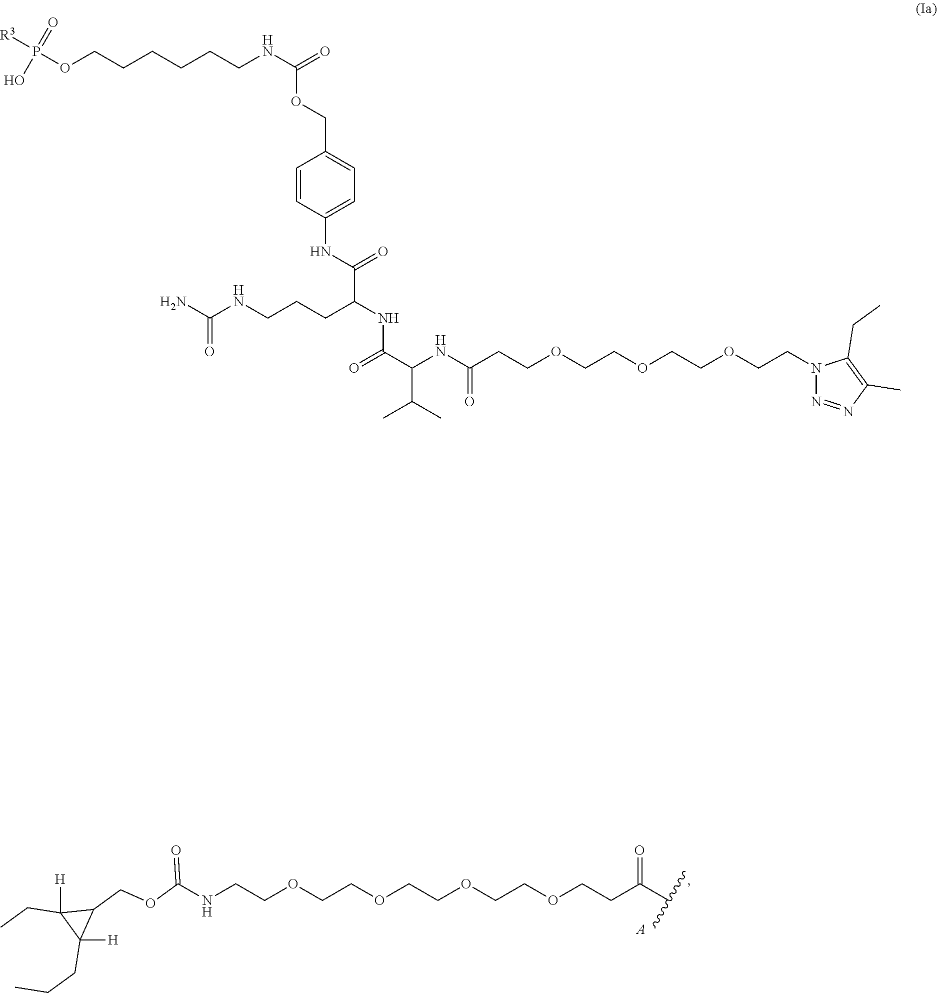

in which R³ comprises an oligonucleotide comprising a nucleobase sequence of CAGCGCCCACCAGUCA (SEQ ID NO: 21) and comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
wherein R² comprises the anti-TfR1 antibody; and
wherein in each complex, n1 is independently an integer of one or greater representing the number of instances of R¹, wherein each instance of R¹ is covalently linked via attachment point A to a different lysine of the anti-TfR1 antibody. In some embodiments, the average value of n1 of the complexes of the composition is in the range of 0.5-5.

In some embodiments, each complex comprises a structure of formula (I): [R¹]ₙ₁—R², wherein:
each R¹ comprises a group of the formula (Ib):

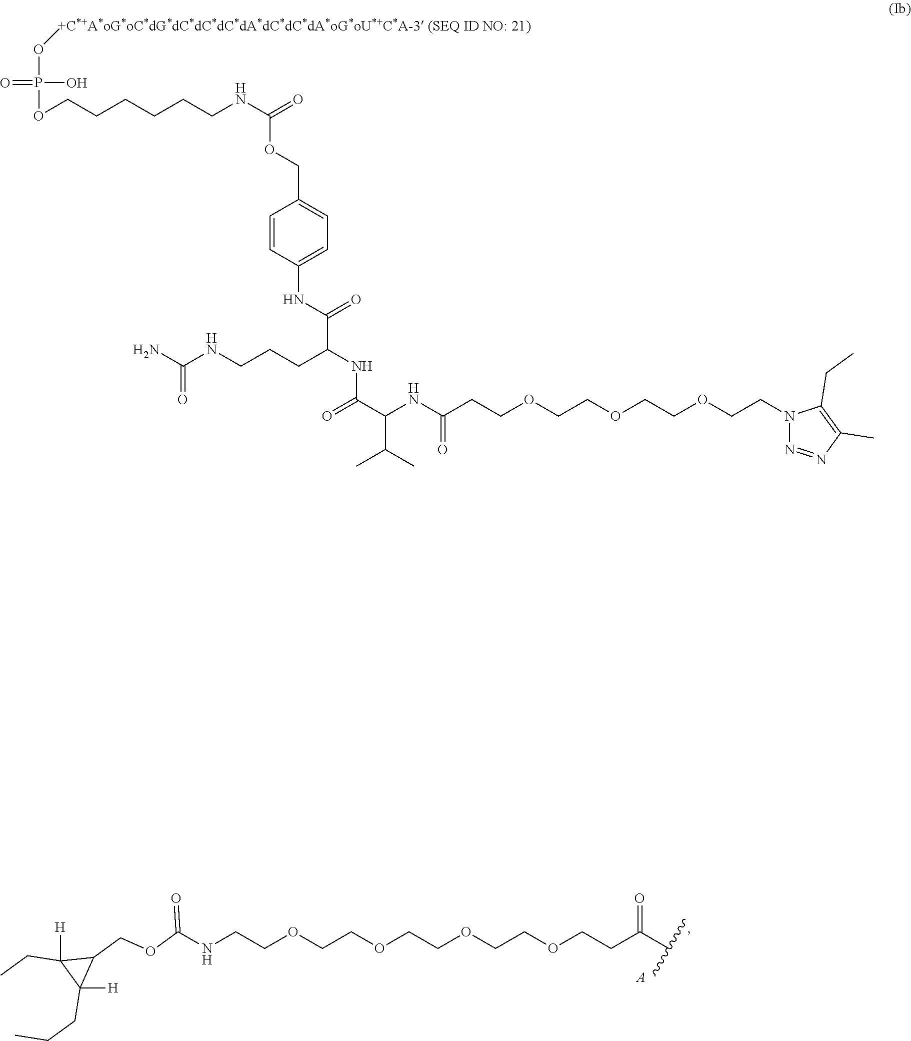

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
wherein R² comprises the anti-TfR1 antibody; and
wherein in each complex, n1 is independently an integer of one or greater representing the number of instances of R¹, wherein each instance of R¹ is covalently linked via attachment point A to a different lysine of the anti-TfR1 antibody. In some embodiments, the average value of n1 of the complexes of the composition is in the range of 0.5-5.

In some embodiments, each complex comprises a structure of formula (I): [R¹]ₙ₁—R², wherein:
each R¹ comprises a group of the formula (Ic):

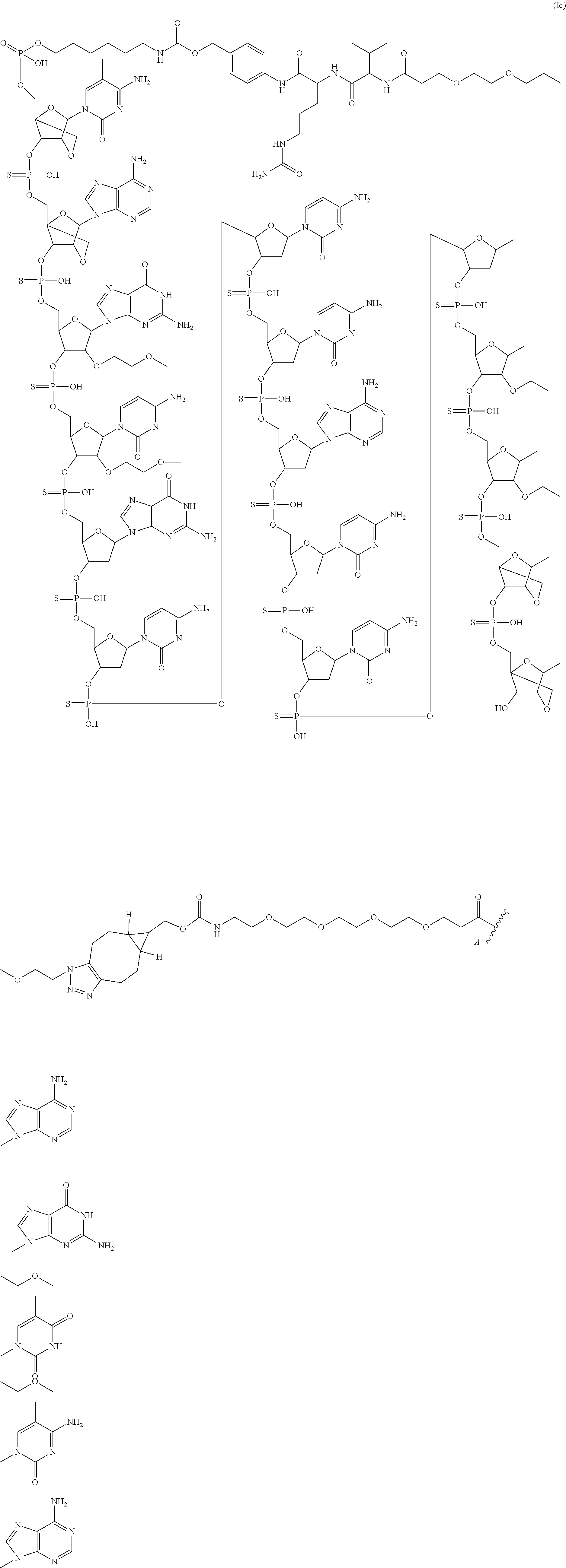

wherein R² comprises the anti-TfR1 antibody; and
wherein in each complex, n1 is independently an integer of one or greater representing the number of instances of R¹, wherein each instance of R¹ is covalently linked via attachment point A to a different lysine of the anti-TfR1 antibody. In some embodiments, the average value of n1 of the complexes of the composition is in the range of 0.5-5.

In some embodiments, each complex comprises a structure of the formula (Id):

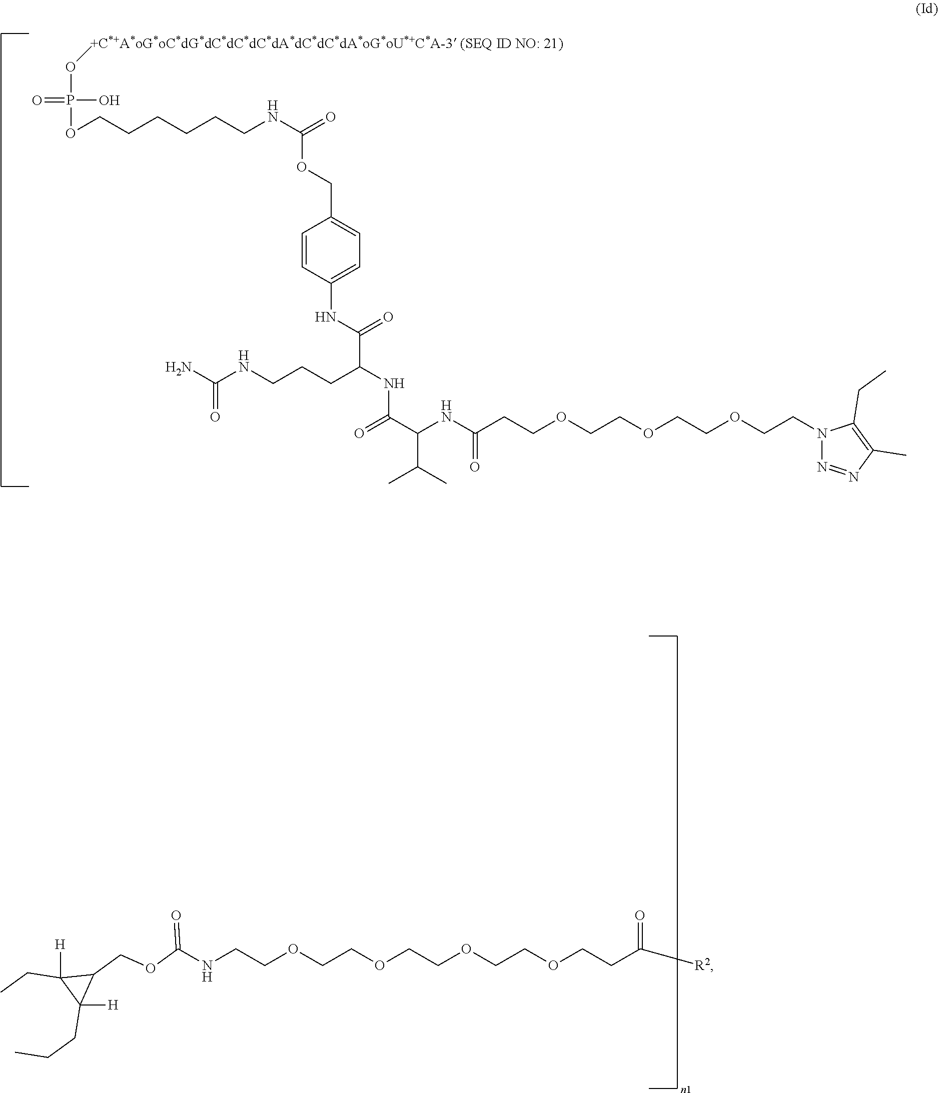

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
wherein R² comprises the anti-TfR1 antibody; and
wherein in each complex, n1 is independently an integer of one or greater representing the number of instances of R¹, wherein each instance of R¹ is covalently linked via attachment point A to a different lysine of the anti-TfR1 antibody. In some embodiments, the average value of n1 of the complexes of the composition is in the range of 0.5-5.

In some embodiments, the anti-TfR1 antibody is a Fab fragment.

In some embodiments, the anti-TfR1 antibody comprises a heavy chain variable region (VH) comprising the amino acid sequence of SEQ ID NO: 17 and a light chain variable region (VL) comprising the amino acid sequence of SEQ ID NO: 18. In some embodiments, the anti-TfR1 antibody comprises a heavy chain comprising the amino acid sequence of SEQ ID NO: 19 and a light chain comprising the amino acid sequence of SEQ ID NO: 20.

In some embodiments, the administration occurs one or more times.

In some embodiments, the effective amount of each administration provides to the subject 10 mg to 110 mg of the anti-TfR1 antibodies of the complexes per kg of the subject.

In some embodiments, the effective amount of each administration provides to the subject 5 mg to 90 mg of the anti-TfR1 antibodies of the complexes per kg of the subject.

In some embodiments, the effective amount of each administration provides to the subject 10 mg to 20 mg of the anti-TfR1 antibodies of the complexes per kg of the subject.

In some embodiments, the effective amount of each administration provides to the subject 13 mg of the anti-TfR1 antibodies of the complexes per kg of the subject.

In some embodiments, the effective amount of each administration provides to the subject 18 mg to 36 mg of the anti-TfR1 antibodies of the complexes per kg of the subject.

In some embodiments, the effective amount of each administration provides to the subject 25 mg of the anti-TfR1 antibodies of the complexes per kg of the subject.

In some embodiments, the effective amount of each administration provides to the subject 36 mg to 72 mg of the anti-TfR1 antibodies of the complexes per kg of the subject.

In some embodiments, the effective amount of each administration provides to the subject 50 mg of the anti-TfR1 antibodies of the complexes per kg of the subject.

In some embodiments, the effective amount of each administration provides to the subject 55 mg to 110 mg of the anti-TfR1 antibodies of the complexes per kg of the subject.

In some embodiments, the effective amount of each administration provides to the subject 75 mg of the anti-TfR1 antibodies of the complexes per kg of the subject.

In some embodiments, the effective amount of each administration provides to the subject 6 mg to 12 mg of the anti-TfR1 antibodies of the complexes per kg of the subject.

In some embodiments, the effective amount of each administration provides to the subject 8 mg of the anti-TfR1 antibodies of the complexes per kg of the subject.

In some embodiments, the effective amount of each administration provides to the subject 11 mg to 22 mg of the anti-TfR1 antibodies of the complexes per kg of the subject.

In some embodiments, the effective amount of each administration provides to the subject 15 mg of the anti-TfR1 antibodies of the complexes per kg of the subject.

In some embodiments, the effective amount of each administration provides to the subject 22 mg to 44 mg of the anti-TfR1 antibodies of the complexes per kg of the subject.

In some embodiments, the effective amount of each administration provides to the subject 30 mg of the anti-TfR1 antibodies of the complexes per kg of the subject.

In some embodiments, the effective amount of each administration provides to the subject 44 mg to 88 mg of the anti-TfR1 antibodies of the complexes per kg of the subject.

In some embodiments, the effective amount of each administration provides to the subject 60 mg of the anti-TfR1 antibodies of the complexes per kg of the subject.

In some embodiments, the effective amount of each administration provides to the subject 20 mg to 43 mg of the anti-TfR1 antibodies of the complexes per kg of the subject. In some embodiments, the effective amount of each administration provides to the subject 29 mg of the anti-TfR1 antibodies of the complexes per kg of the subject.

In some embodiments, the effective amount of each administration provides to the subject 26 mg to 53 mg of the anti-TfR1 antibodies of the complexes per kg of the subject.

In some embodiments, the effective amount of each administration provides to the subject 37 mg of the anti-TfR1 antibodies of the complexes per kg of the subject.

In some embodiments, during a period of administration, the composition is administered once every 4 weeks, once every 8 weeks, or once every 12 weeks. In some embodiments, the period of administration is less than 10 years. In some embodiments, the period of administration is the remainder of the subject's lifetime.

In some embodiments, the composition is administered once every 4 weeks during a first period of administration, and subsequently administered once every 8 weeks during a second period of administration. In some embodiments, the first period of administration is 8-16 weeks, and/or the second period of administration is 16 weeks to the remainder of the subject's lifetime.

In some embodiments, the composition is in the form of an aqueous solution and further comprises tris(hydroxymethyl)aminomethane and sucrose. In some embodiments, the tris(hydroxymethyl)aminomethane is present in the aqueous solution at a concentration of 25 mM, the sucrose is present in the aqueous solution at a concentration of 10 w/v %, and the aqueous solution is at a pH of 7.5. In some embodiments, the complexes are present in the composition at a concentration in the range of 10 mg/mL to 50 mg/mL.

In some embodiments, the administering reduces DMPK expression in a muscle cell of the subject. In some embodiments, reducing DMPK expression comprises reducing the amount of DMPK RNA in the muscle cell. In some embodiments, the DMPK RNA amount is reduced in the nucleus of the muscle cell.

In some embodiments, reducing DMPK expression in the muscle cell comprises reducing the amount of DMPK protein in the muscle cell.

In some embodiments, the subject is human.

In some embodiments, the complex is administered systemically. In some embodiments, the complex is administered intravenously. In some embodiments, the complex is administered by infusion.

In some embodiments, the composition further comprises one or more anti-TfR1 antibodies that are not covalently linked to an oligonucleotide.

### BRIEF DESCRIPTION OF THE DRAWINGS

FIGS. 1A-1D show the amount of DMPK-targeting oligonucleotide (ASO) in the heart (FIG. 1A), diaphragm (FIG. 1B), gastrocnemius (FIG. 1C), or tibialis anterior (FIG. 1D), respectively, after administration of conjugates containing an anti-TfR1 Fab covalently linked to the ASO.

FIGS. 2A-2D show the ability of conjugates containing an anti-TfR1 Fab conjugated to a DMPK-targeting oligonucleotide (ASO) to knock down human DMPK RNA in the heart (FIG. 2A), diaphragm (FIG. 2B), tibialis anterior (FIG. 2C) and gastrocnemius (FIG. 2D) of mice expressing both human TfR1 and two copies of a mutant human DMPK transgene that harbors expanded CTG repeats.

FIGS. 3A-3B show reduced DMPK foci in nuclei of cardiac muscle fibers in mice expressing both human TfR1 and two copies of a mutant human DMPK transgene that harbors expanded CTG repeats and treated with anti-TfR1 Fab conjugated to DMPK-targeting oligonucleotide (ASO). FIG. 3A shows representative images of samples following in situ hybridization staining for DMPK foci and fluorescence staining of myofibers (inset panels). In the microscopy images shown in FIG. 3A, the light rounded shapes show cell nuclei, and the bright puncta within the nuclei show DMPK foci. FIG. 3B shows quantification of DMPK foci.

FIG. 4 shows the splicing correction activity of conjugates containing an anti-TfR1 Fab covalently linked to a DMPK-targeting oligonucleotide (ASO) in the heart of mice expressing both human TfR1 and two copies of a mutant human DMPK transgene that harbors expanded CTG repeats (hTfR1/DMSXL mice). Composite splicing indices based on splicing of Ldb3 exon 11, Mbnl2 exon 6, and Nfix exon 7 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 5 shows the splicing correction activity of conjugates containing an anti-TfR1 Fab covalently linked to a DMPK-targeting oligonucleotide (ASO) in the diaphragm of mice expressing both human TfR1 and two copies of a mutant human DMPK transgene that harbors expanded CTG repeats (hTfR1/DMSXL mice). Composite splicing indices based on splicing of Bin1 exon 11, Insr exon 11, Ldb3 exon 11 and Nfix exon 7 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 6 shows the splicing correction activity of conjugates containing an anti-TfR1 Fab covalently linked to a DMPK-targeting oligonucleotide (ASO) in the tibialis anterior of mice expressing both human TfR1 and two copies of a mutant human DMPK transgene that harbors expanded CTG repeats (hTfR1/DMSXL mice). Composite splicing indices based on splicing of Bin1 exon 11, Ldb3 exon 11, Mbnl2 exon 6, and Nfix exon 7 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 7 shows the splicing correction activity of conjugates containing an anti-TfR1 Fab covalently linked to a DMPK-targeting oligonucleotide (ASO) in the gastrocnemius of mice expressing both human TfR1 and two copies of a mutant human DMPK transgene that harbors expanded CTG repeats (hTfR1/DMSXL mice). Composite splicing indices based on splicing of Mbnl2 exon 6, Nfix exon 7, and Ttn exon 313 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 8 shows DMPK knockdown in DM1 patient myotubes and wild-type non-human primate (NHP) myotubes resulting from incubation with conjugates containing an anti-TfR1 Fab covalently linked to a DMPK-targeting oligonucleotide (ASO). Results are shown normalized to expression in DM1 patient myotubes or NHP myotubes treated with vehicle only. Data are shown as mean+standard deviation for n=4 replicates per condition. Statistics were calculated by one-way ANOVA (*, P<0.05, **, P<0.01).

FIGS. 9A-9D show DMPK expression in the heart (FIG. 9A), diaphragm (FIG. 9B), tibialis anterior (FIG. 9C), or gastrocnemius (FIG. 9D), respectively, of mice over 12 weeks after administration of conjugates containing an anti-TfR1 Fab covalently linked to a DMPK-targeting ASO. DMPK expression is relative to expression in vehicle-treated animals. Data are shown as means±standard deviation for n=5−12 replicates per tissue. Statistics were calculated by ANOVA followed by uncorrected Fisher's Least Significant Difference test (*, P<0.05; **, P<0.01; ***, P<0.001; ****, P<0.0001).

FIGS. 10A-10D show tissue DMPK expression in the heart (FIG. 10A), diaphragm (FIG. 10B), tibialis anterior (FIG. 10C), or gastrocnemius (FIG. 10D), respectively, of mice after administration of conjugates containing an anti-TfR1 Fab covalently linked to a DMPK-targeting ASO. The conjugates were administered as either one 10 mg/kg ASO-equivalent dose (“10”), two 5 mg/kg ASO-equivalent doses one week apart (“2×5 Weekly”), or two 5 mg/kg ASO-equivalent doses two weeks apart (“2×5 Every 2 weeks”). Tissue DMPK levels were measured four weeks after administration of the final dose and are presented relative to vehicle-treated controls (“Vehicle”). Data are shown as means+standard deviation for n=3-6 replicates per tissue. Statistics were calculated by one-way ANOVA (*, P<0.05; **, P<0.01; ***, P<0.001; ****, P<0.0001).

FIGS. 11A-11D show DMPK expression in the heart (FIG. 11A), diaphragm (FIG. 11B), tibialis anterior (FIG. 11C), or gastrocnemius (FIG. 11D), respectively, of mice after administration of varying dosages of conjugates containing an anti-TfR1 Fab covalently linked to a DMPK-targeting ASO. The conjugates were administered as either one 5 mg/kg ASO-equivalent dose (“5”), one 10 mg/kg ASO-equivalent dose (“10”), or one 20 mg/kg ASO-equivalent dose (“20”), four weeks after which tissue DMPK levels were measured. Data are shown as means+standard deviation and are presented relative to vehicle-treated controls (“Vehicle”). Statistics were calculated by one-way ANOVA with Dunnet's post-hoc analysis (*, P<0.05; **, P<0.01; ***, P<0.001).

FIGS. 12A-12D show DMPK expression in the heart (FIG. 12A), diaphragm (FIG. 12B), tibialis anterior (FIG. 12C), or gastrocnemius (FIG. 12D), respectively, of mice after administration varying dosages of conjugates containing an anti-TfR1 Fab covalently linked to a DMPK-targeting ASO. The conjugates were administered as either two 5 mg/kg ASO-equivalent doses administered one week apart (“2×5”) or two 10 mg/kg ASO-equivalent doses administered one week apart (“2×10”). Tissue DMPK levels were measured four weeks after administration of the first dose and are presented relative to vehicle-treated controls (“Vehicle”). Data are shown as means+standard deviation for n=5-6 replicates per tissue. Statistics were calculated by Brown-Forsythe and Welch ANOVA test with Dunnett's T3 multiple comparisons test (*, P<0.05; **, P<0.01; ****, P<0.0001).

FIGS. 13A-13D show DMPK expression in the heart (FIG. 13A), diaphragm (FIG. 13B), tibialis anterior (FIG. 13C), or gastrocnemius (FIG. 13D), respectively, of mice after administration of varying dosages of conjugates containing an anti-TfR1 Fab covalently linked to a DMPK-targeting ASO. The conjugates were administered as four 5 mg/kg ASO-equivalent doses (“4×5”) or four 10 mg/kg ASO-equivalent doses (“4×10”), each dose administered four weeks apart (on days 0, 28, 56, and 84, respectively). Four weeks after the final dose was administered (16 weeks following administration of the first dose), tissue DMPK levels were measured and are presented relative to vehicle-treated controls (“Vehicle”). Data are shown as means+standard deviation (n=6 mice per group). Statistics were calculated by one-way ANOVA followed by uncorrected Fisher's Least Significant Difference (LSD) test (*, P<0.05; **, P<0.01; ***, P<0.001; ****, P<0.0001).

FIGS. 14A-14B show DMPK expression the gastrocnemius (FIG. 14A) and tibialis anterior (FIG. 14B), respectively, of non-human primates after administration of 10 mg/kg ASO-equivalent of conjugates containing an anti-TfR1 Fab covalently linked to a DMPK-targeting ASO. Tissue DMPK levels were measured 4, 8 and 12 weeks after administration of the conjugates and are presented relative to vehicle-treated controls. Data are shown as means±standard deviation. Statistics were calculated by unpaired t-test (*, P<0.05).

FIGS. 15A-15D show DMPK expression the heart (FIG. 15A), diaphragm (FIG. 15B), tibialis anterior (FIG. 15C), or gastrocnemius (FIG. 15D), respectively, of non-human primates after administration of 5 mg/kg (“5”) or 10 mg/kg (“10”) ASO-equivalent of conjugates containing an anti-TfR1 Fab covalently linked to a DMPK-targeting ASO. Following administration of either a single 5 mg/kg ASO-equivalent dose or a single 10 mg/kg ASO-equivalent dose to a non-human primate model, tissue DMPK levels were measured after four weeks and are presented relative to vehicle-treated controls (“Vehicle”). Data are shown as means+standard deviation. Statistics were calculated by one-way ANOVA (*, P<0.05; **, P<0.01).

FIGS. 16A-16G show DMPK expression in the heart (FIG. 16A), diaphragm (FIG. 16B), tibialis anterior (FIG. 16C), gastrocnemius (FIG. 16D), masseter (FIG. 16E), esophagus (FIG. 16F), or duodenum (FIG. 16G) respectively, of non-human primates after administration of either two 5 mg/kg ASO-equivalent doses (“2×5”) or two 10 mg/kg ASO-equivalent doses (“2×10”) of conjugates containing an anti-TfR1 Fab covalently linked to a DMPK-targeting ASO. Following administration of either two 5 mg/kg ASO-equivalent doses or two 10 mg/kg ASO-equivalent doses to a non-human primate model, each four weeks apart, tissue DMPK levels were measured four weeks after the final dose and are presented relative to vehicle-treated controls (“Vehicle”). Data are shown as means+standard deviation. Statistics were calculated by one-way ANOVA followed by an uncorrected Fisher's Least Significant Difference (LSD) test (*, P<0.05; **. P<0.01; ***. P<0.001).

### DETAILED DESCRIPTION OF INVENTION

According to some aspects, the present disclosure provides methods of reducing expression or activity of DMPK (e.g., reducing the level of a mutant or wild-type DMPK RNA) and/or methods of treating myotonic dystrophy (e.g., DM1) in a subject. In some embodiments, the methods described herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 0.5 mg to 20 mg (e.g., about 1 mg to about 12 mg, about 1.8 mg, about 3.4 mg, about 6.8 mg, or about 10.2 mg) of the oligonucleotides of the complexes per kg of the subject. In some embodiments, the effective amount provides to the subject about 4 mg or about 5 mg of the oligonucleotides of the complexes per kg of the subject. In some embodiments, the effective amount provides to the subject 2 mg to 220 mg (e.g., about 5 mg to about 110 mg, about 13 mg, about 25 mg, about 50 mg, or about 75 mg) of the anti-TfR1 antibody (e.g., Fab) of the complexes per kg of the subject. In some embodiments, the effective amount provides to the subject about 29 mg or about 37 mg of the anti-TfR1 antibody (e.g., Fab) of the complexes per kg of the subject. In some embodiments, the administration is once every two weeks to once every 12 weeks (e.g., once every two weeks, once every four weeks, once every 8 weeks, or once every 12 weeks). In some embodiments, the subject has a DMPK allele associated with DM1 (e.g., wherein the DMPK allele comprises a DM1 disease-associated repeat expansion).

Further aspects of the disclosure, including a description of defined terms, are provided below.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M. P. et al., Nucleic Acids Res., 27:209-212 (1999); Ruiz, M. et al., Nucleic Acids Res., 28:219-221 (2000); Lefranc, M. P., Nucleic Acids Res., 29:207-209 (2001); Lefranc, M. P., Nucleic Acids Res., 31:307-310 (2003); Lefranc, M. P. et al., In Silico Biol., 5, 0006 (2004) [Epub], 5:45-60 (2005); Lefranc, M. P. et al., Nucleic Acids Res., 33:D593-597 (2005); Lefranc, M. P. et al., Nucleic Acids Res., 37:D1006-1012 (2009); Lefranc, M. P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M. -P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Disease-associated-repeat: As used herein, the term “disease-associated-repeat” refers to a repeated nucleotide sequence at a genomic location for which the number of units of the repeated nucleotide sequence is correlated with and/or (e.g., and) directly or indirectly contributes to, or causes, genetic disease. Each repeating unit of a disease associated repeat may be 2, 3, 4, 5 or more nucleotides in length. For example, in some embodiments, a disease associated repeat is a dinucleotide repeat. In some embodiments, a disease associated repeat is a trinucleotide repeat. In some embodiments, a disease associated repeat is a tetranucleotide repeat. In some embodiments, a disease associated repeat is a pentanucleotide repeat. In some embodiments, the disease-associated-repeat comprises CAG repeats, CTG repeats, CUG repeats, CGG repeats, CCTG repeats, or a nucleotide complement of any thereof. In some embodiments, a disease-associated-repeat is in a non-coding portion of a gene. However, in some embodiments, a disease-associated-repeat is in a coding region of a gene. In some embodiments, a disease-associated-repeat is expanded from a normal state to a length that directly or indirectly contributes to, or causes, genetic disease. In some embodiments, a disease-associated-repeat is in RNA (e.g., an RNA transcript). In some embodiments, a disease-associated-repeat is in DNA (e.g., a chromosome, a plasmid). In some embodiments, a disease-associated-repeat is expanded in a chromosome of a germline cell. In some embodiments, a disease-associated-repeat is expanded in a chromosome of a somatic cell. In some embodiments, a disease-associated-repeat is expanded to a number of repeating units that is associated with congenital onset of disease. In some embodiments, a disease-associated-repeat is expanded to a number of repeating units that is associated with childhood onset of disease. In some embodiments, a disease-associated-repeat is expanded to a number of repeating units that is associated with adult onset of disease. In DM1, the DMPK gene comprises a disease-associated repeat of CTG units.

DMPK: As used herein, the term “DMPK” refers to a gene that encodes myotonin-protein kinase (also known as myotonic dystrophy protein kinase or dystrophia myotonica protein kinase), a serine/threonine protein kinase. Substrates for this enzyme may include myogenin, the beta-subunit of the L-type calcium channels, and phospholemman. In some embodiments, DMPK may be a human (Gene ID: 1760), non-human primate (e.g., Gene ID: 456139, Gene ID: 715328, Gene ID: 102125829), or rodent gene (e.g., Gene ID: 13400). In humans, a CTG repeat expansion in the 3′ non-coding, untranslated region of DMPK is associated with myotonic dystrophy type I (DM1). In addition, multiple human transcript variants (e.g., as annotated under GenBank RefSeq Accession Numbers: NM_001081563.2, NM_004409.4, NM_001081560.2, NM_001081562.2, NM_001288764.1, NM_001288765.1, and NM_001288766.1) have been characterized that encode different protein isoforms.

DMPK allele: As used herein, the term “DMPK allele” refers to any one of alternative forms (e.g., wild-type or mutant forms) of a DMPK gene. In some embodiments, a DMPK allele may encode for wild-type myotonin-protein kinase that retains its normal and typical functions. In some embodiments, a DMPK allele may comprise one or more disease-associated-repeat expansions. In some embodiments, normal subjects have two DMPK alleles comprising in the range of 5 to 37 repeat units. In some embodiments, the number of CTG repeat units in subjects having DM1 is in the range of about 50 to about 3,000 or more, with higher numbers of repeats leading to an increased severity of disease. In some embodiments, mildly affected DM1 subjects have at least one DMPK allele having in the range of 50 to 150 repeat units. In some embodiments, subjects with classic DM1 have at least one DMPK allele having in the range of 100 to 1,000 or more repeat units. In some embodiments, subjects having DM1 with congenital onset may have at least one DMPK allele comprising more than 2,000 repeat units.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Myotonic dystrophy (DM): As used herein, the term “Myotonic dystrophy (DM)” refers to a genetic disease caused by mutations in the DMPK gene or CNBP (ZNF9) gene that is characterized by muscle loss, muscle weakening, and muscle function. Two types of the disease, myotonic dystrophy type 1 (DM1) and myotonic dystrophy type 2 (DM2), have been described. DM1 is associated with an expansion of a CTG trinucleotide repeat in the 3′ non-coding region of DMPK. DM2 is associated with an expansion of a CCTG tetranucleotide repeat in the first intron of ZNF9. In both DM1 and DM2, the nucleotide expansions lead to toxic RNA repeats capable of forming hairpin structures that bind critical intracellular proteins, e.g., muscleblind-like proteins, with high affinity. Myotonic dystrophy, the genetic basis for the disease, and related symptoms are described in the art (see, e.g. Thornton, C. A., “Myotonic Dystrophy” Neurol Clin. (2014), 32(3): 705-719; and Konieczny et al. “Myotonic dystrophy: candidate small molecule therapeutics” Drug Discovery Today (2017), 22:11). In some embodiments, subjects are born with a variation of DM1 called congenital myotonic dystrophy. Symptoms of congenital myotonic dystrophy are present from birth and include weakness of all muscles, breathing problems, clubfeet, developmental delays and intellectual disabilities. DM1 is associated with Online Mendelian Inheritance in Man (OMIM) Entry #160900. DM2 is associated with OMIM Entry #602668.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a disease-associated-repeat expansion, e.g., in a DMPK allele.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleotides and oligonucleotides comprising the 2′-modified nucleosides have increased affinity to a target sequences, relative to an unmodified oligonucleotide. Examples of structures of 2′-modified nucleosides are provided below:

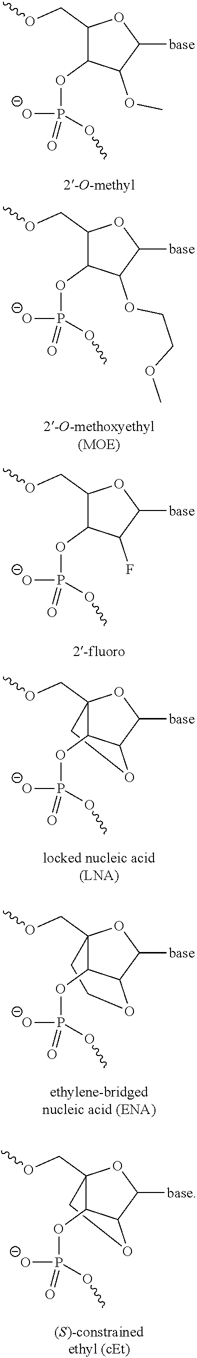

These examples are shown with phosphate groups, but any internucleoside linkages are contemplated between 2′-modified nucleosides.

Ranges: All ranges provided in the present disclosure are inclusive of the end points.

### Complexes

Provided herein are complexes that comprise a targeting agent, e.g., an antibody, covalently linked to an oligonucleotide. In some embodiments, a complex comprises a muscle-targeting antibody covalently linked to one or more oligonucleotides. In some embodiments, the oligonucleotide is an antisense oligonucleotide that targets a DMPK RNA to reduce expression or activity of DMPK (e.g., reduce the level of a mutant or wild-type DMPK RNA, or the activity of a DMPK gene product).

Complexes described herein generally comprise a linker that covalently links an antibody (e.g., an anti-TfR1 antibody) described herein to an oligonucleotide (e.g., an oligonucleotide comprising a 5′-X—Y—Z-3′ configuration). A linker comprises at least one covalent bond.

In some embodiments, complexes described herein comprise a structure of formula (I): [R¹]ₙ₁—R², in which each R¹ independently comprises a compound comprising an oligonucleotide (e.g., an oligonucleotide comprising a 5′-X—Y—Z-3′ configuration) and R² comprises an antibody (e.g., an anti-TfR1 antibody), and wherein in each complex n1 is independently an integer (e.g., one or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² comprises an antibody (e.g., an anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R².

In some embodiments, in each complex n1 is independently an integer (e.g., one or greater). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv. In some embodiments, the antibody is a Fab fragment.

In some embodiments, the value of n1 of each or any complex (e.g., any complex in any of the compositions or formulations disclosed herein) is an integer up to the number of amino acid residues in the antibody to which conjugation is desired or targeted (e.g., the number of lysine residues). In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex the value of n1 is independently in the range of 1-27, 1-26, 1-10, 1-5, or 1-3. In some embodiments, the average value of n1 of complexes of the composition is in the range of 1 to 5 (e.g., 1-5, 1-4, 1-3, 3-5, or 1-2). In some embodiments, compositions described herein comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, compositions are provided (e.g., formulations comprising tris(hydroxymethyl)aminomethane and/or sucrose, as described herein) that comprise a plurality of different complexes. In some embodiments, the plurality of different complexes comprise a common targeting agent (e.g. an antibody) and a common oligonucleotide (e.g., an oligonucleotide comprising a 5′-X—Y—Z-3′ configuration, such as a DMPK-targeting oligonucleotide). In such embodiments, different complex types are characterized by having different numbers of oligonucleotides covalently linked to an antibody. For example, in some embodiments, compositions are provided that comprise a plurality of complexes comprising a structure of formula (I): [R¹]ₙ₁—R², in which each R¹ independently comprises a compound comprising an oligonucleotide (e.g., a DMPK-targeting oligonucleotide) and R² comprises an antibody (e.g., anti-TfR1 antibody), and in which n1 is an integer representing the number of instances of R¹ in a complex, and in which different complexes of the composition may have different n1 values (e.g., n1 values in the range of 1-27, 1-26, 1-10, 1-5, or 1-3). In some embodiments, in complexes of a composition n1 is independently an integer. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, compositions described herein comprise complexes in which n1 is 0.

In some embodiments, a composition described herein comprises antibody that is not conjugated to an oligonucleotide (e.g., in trace amounts) and antibody conjugated to one or more oligonucleotides. In some embodiments, antibody that is not conjugated to an oligonucleotide may be referred to as a compound of the structure of formula (I): [R¹]ₙ₁—R², for which n1 is zero. Accordingly, in some embodiments, a composition for administration to a subject in the methods described herein comprises compounds (e.g., complexes) of the structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds of the structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, each instance of R¹ in a complex is covalently linked to a different amino acid residue of the antibody. In some embodiments, an amino acid to which R¹ is covalently linked comprises an F-amino group (e.g., lysine, arginine). However, in some embodiments, an amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, R¹ is not covalently linked to an amino acid residue residing in a CDR region of the antibody.

In some embodiments, complexes provided herein (e.g., in compositions or formulations described herein) comprise a structure of formula (I): [R¹]ₙ₁—R², in which each instance of R¹ independently comprises a group of the formula (Ia):

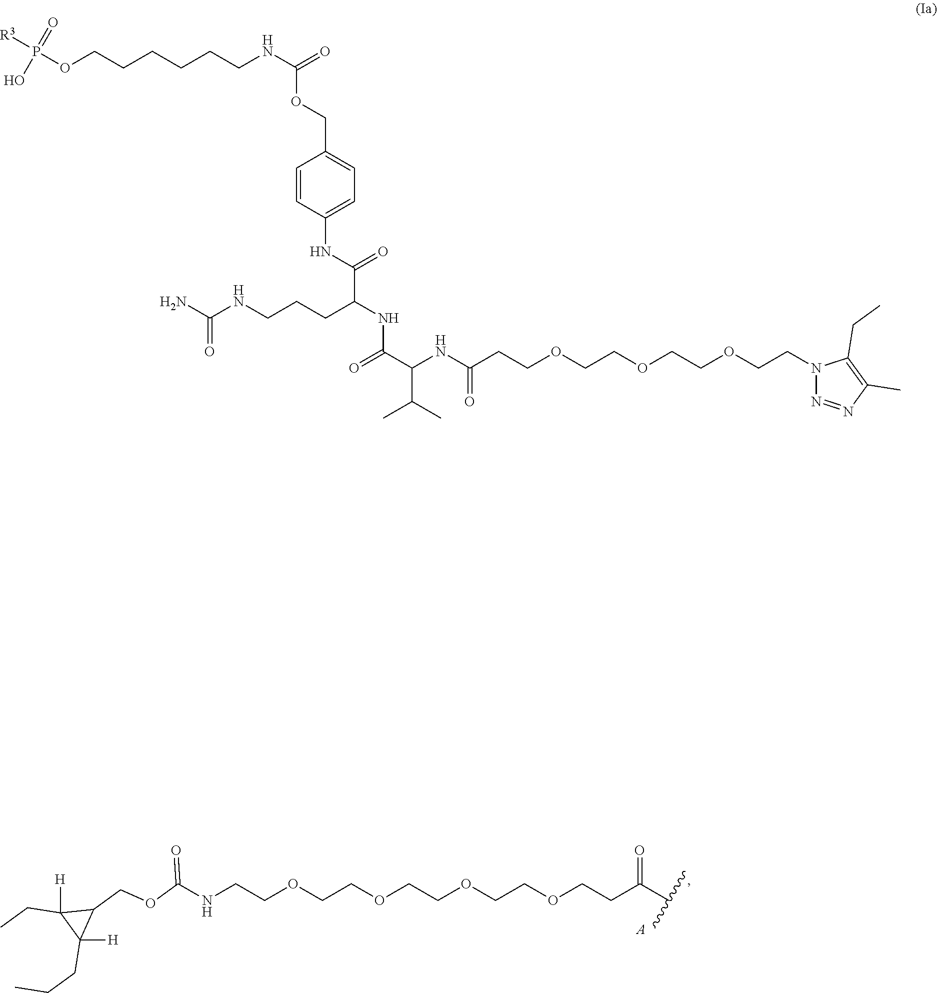

in which R³ comprises an oligonucleotide, e.g., an oligonucleotide comprising a 5′-X—Y—Z-3′ configuration; and R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R³ comprises an oligonucleotide comprising a nucleobase sequence of CAGCGCCCACCAGUCA (SEQ ID NO: 21). In some embodiments, R³ comprises an oligonucleotide comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage.

In some embodiments, complexes provided herein (e.g., in compositions or formulations described herein) comprise a structure of formula (I): [R¹]ₙ₁—R², in which each R¹ comprises a group of the formula (Ib):

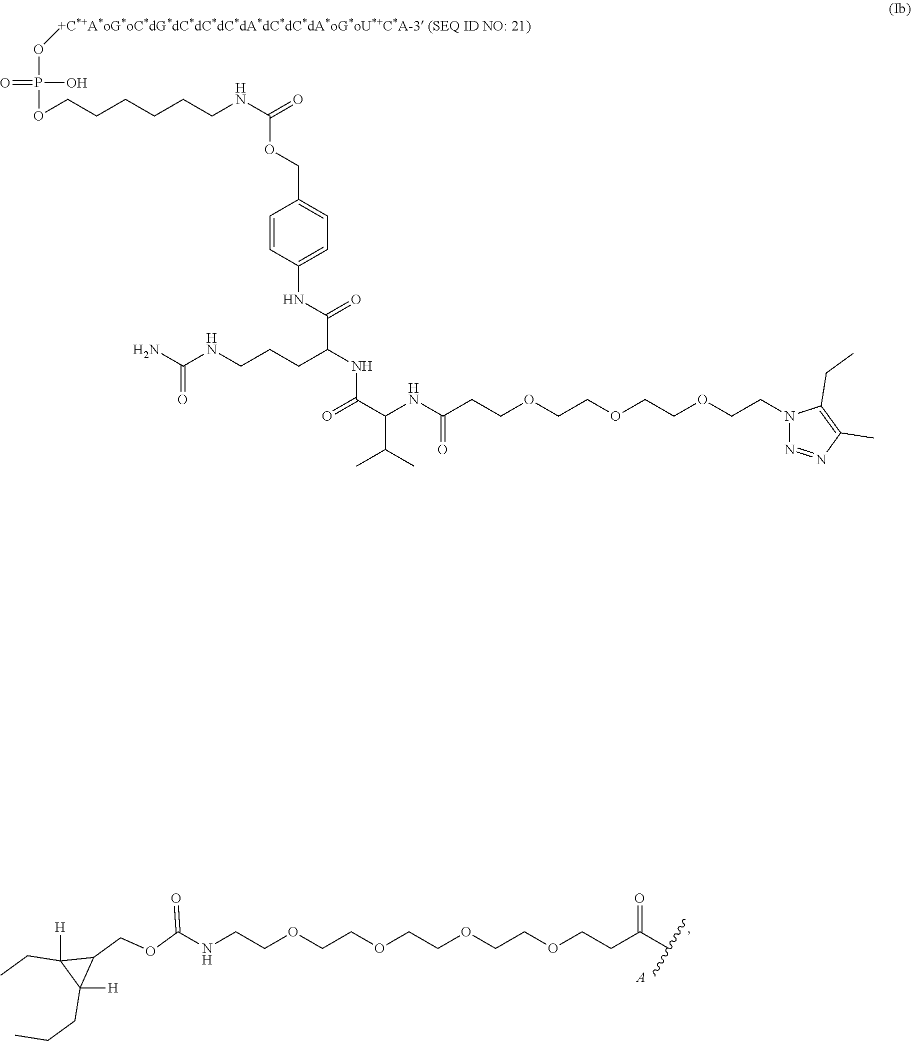

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21), wherein n1 is an integer (e.g., one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv.

In some embodiments, complexes provided herein (e.g., in compositions or formulations described herein) comprise a structure of formula (I): [R¹]ₙ₁— R², in which each R¹ comprises a group of the formula (Ic):

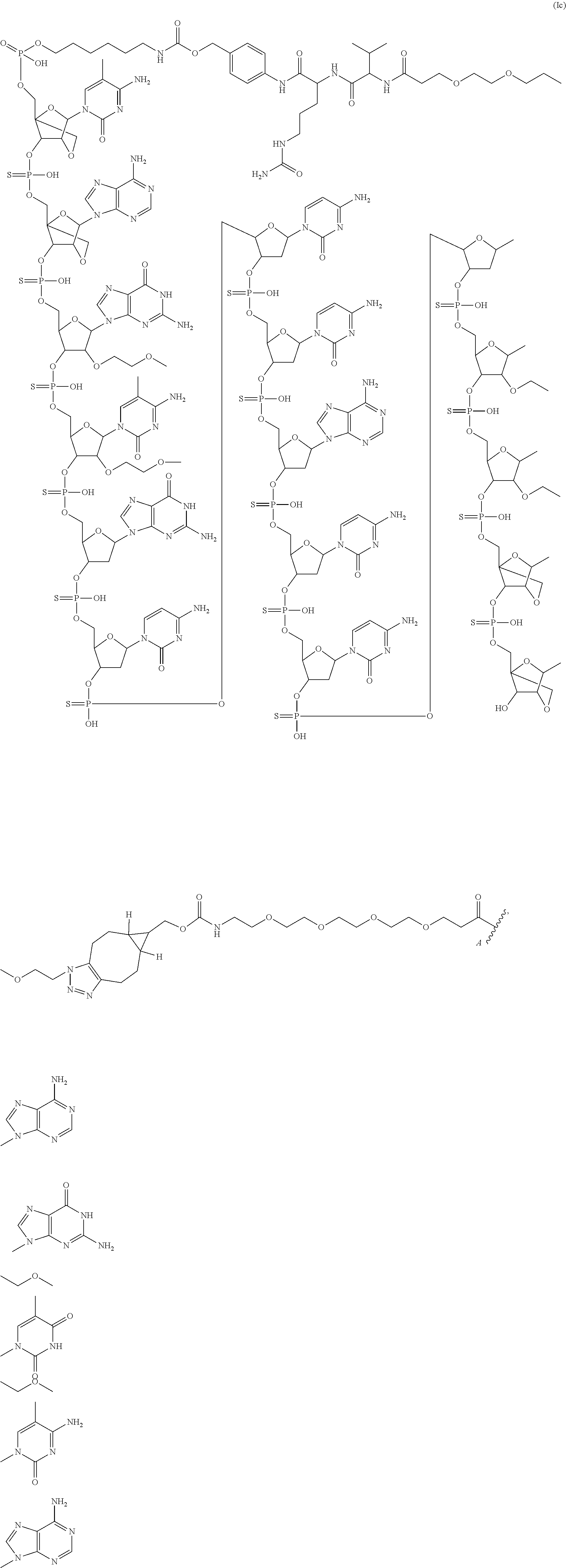

wherein R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv.

In some embodiments, complexes provided herein (e.g., in compositions or formulations described herein) comprise a structure of the formula (Id):

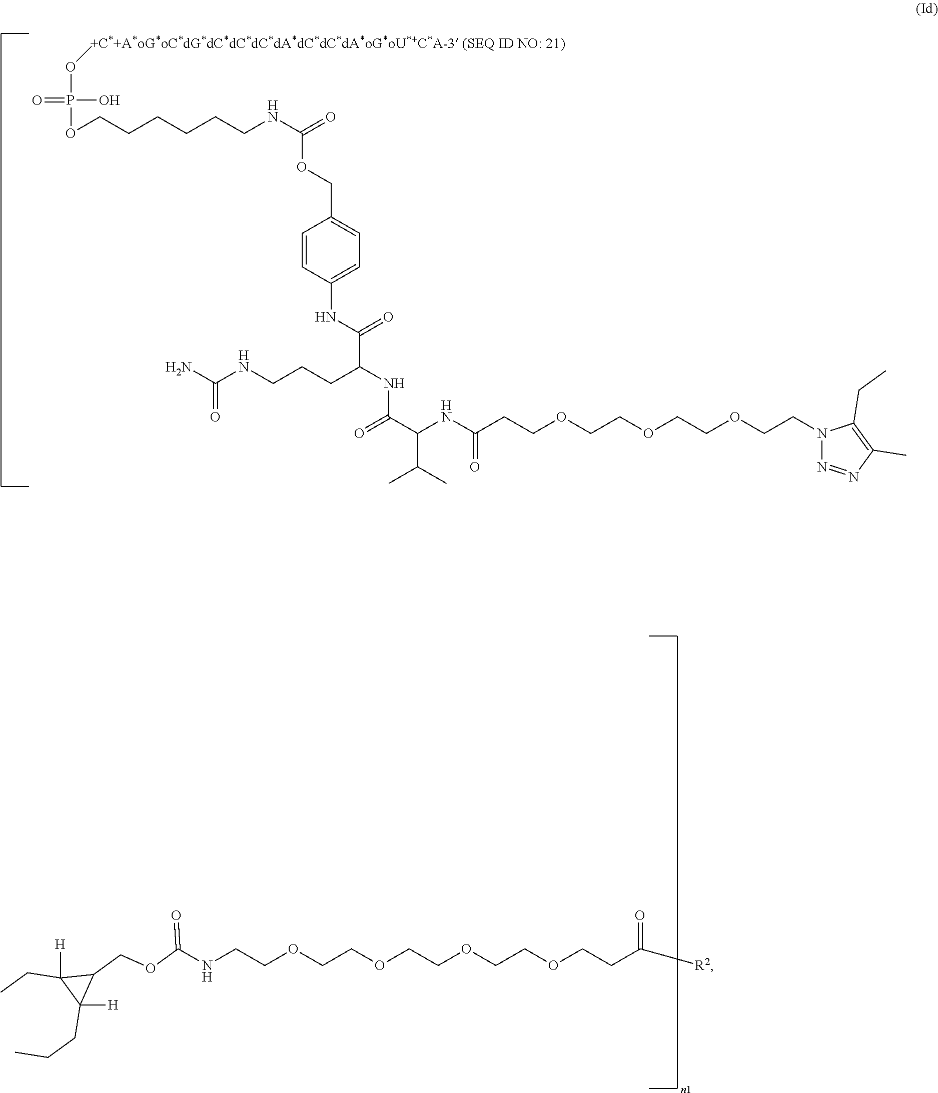

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21); wherein R² comprises an antibody comprising a sequence as set forth in Table 2; wherein n1 is an integer (e.g., one or greater) representing the number of instances of the group enclosed by square brackets, wherein each instance of the group enclosed by square brackets is covalently linked to a different amino acid residue of the antibody, optionally wherein each different amino acid residue is a lysine. In some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv.

In some embodiments, complexes described herein comprise a structure of formula (A):

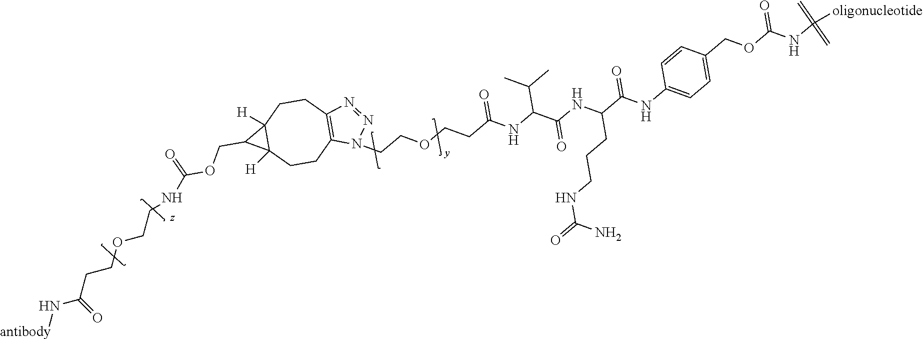

(A), wherein y is 0-15 (e.g., 3) and z is 0-15 (e.g., 4). In some embodiments, the amide shown adjacent the antibody (e.g., anti-TfR1 antibody) in the structure (A) results from a reaction with an amine of the antibody, such as a lysine epsilon amine. In some embodiments, a complex described herein comprises an anti-TfR1 antibody (e.g., an anti-TfR1 Fab) covalently linked via a lysine of the antibody to the 5′ end of an oligonucleotide (e.g., an oligonucleotide comprising a 5′-X—Y—Z-3′ configuration). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

### Antibodies

In some embodiments, complexes described herein comprise an antibody that binds human transferrin receptor 1 (TfR1). An example human TfR1 amino acid sequence, corresponding to NCBI sequence NP_003225.2 (transferrin receptor protein 1 isoform 1, Homo sapiens) is as follows:

(SEQ ID NO: 23)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

Table 2 provides examples of sequences of an anti-TfR1 antibody useful in the complexes provided herein.

TABLE 2
Examples of anti-TfR1 antibody sequences

| Feature | IMGT | Kabat | Chothia |
|---|---|---|---|
| CDR-H1 | GYSITSGYY | SGYYWN (SEQ ID | GYSITSGY |
| | (SEQ ID NO: 1) | NO: 7) | (SEQ ID NO: 12) |
| CDR-H2 | ITFDGAN | YITFDGANNYNPSL | FDG |
| | (SEQ ID NO: 2) | KN (SEQ ID NO: 8) | (SEQ ID NO: 13) |
| CDR-H3 | TRSSYDYDVL | SSYDYDVLDY | SYDYDVLD |
| | DY (SEQ ID | (SEQ ID NO: 9) | (SEQ ID NO: 14) |
| | NO: 3) | | |
| CDR-L1 | QDISNF (SEQ | RASQDISNFLN | SQDISNF |
| | ID NO: 4) | (SEQ ID NO: 10) | (SEQ ID NO: 15) |
| CDR-L2 | YTS (SEQ ID | YTSRLHS (SEQ ID | YTS |
| | NO: 5) | NO: 11) | (SEQ ID NO: 5) |
| CDR-L3 | QQGHTLPYT | QQGHTLPYT (SEQ | GHTLPY |
| | (SEQ ID NO: 6) | ID NO: 6) | (SEQ ID NO: 16) |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. Alternatively or in addition (e.g., in addition), the anti-TfR1 antibody of the present disclosure comprises a light chain comprising the amino acid sequence of SEQ ID NO: 20. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Oligonucleotides

In some embodiments, an oligonucleotide of the complexes described herein is a single stranded oligonucleotide. In some embodiments, the oligonucleotide is useful for targeting DMPK (e.g., for reducing expression or activity of a DMPK RNA, such as the level of a mutant or wild-type DMPK RNA). In some embodiments, an oligonucleotide that is useful for targeting DMPK RNAs. (e.g., for reducing expression or activity of a DMPK RNA, such as the level of a mutant or wild-type DMPK RNA). In some embodiments, the oligonucleotide comprises a region of complementarity to a DMPK RNA. In some embodiments, the oligonucleotide is useful for reducing levels of toxic DMPK having disease-associated repeat expansions, e.g., in a subject having or suspected of having myotonic dystrophy. In some embodiments, the oligonucleotide is designed to direct RNAse H mediated degradation of the target DMPK RNA residing in the nucleus of cells, e.g., muscle cells (e.g., myotubes) or cells of the nervous system (e.g., central nervous system (CNS) cells). In some embodiments, the oligonucleotide is designed to have desirable bioavailability and/or serum-stability properties. In some embodiments, the oligonucleotide is designed to have desirable binding affinity properties. In some embodiments, the oligonucleotide is designed to have desirable toxicity profiles. In some embodiments, the oligonucleotide is designed to have low-complement activation and/or cytokine induction properties.

In some embodiments, DMPK-targeting oligonucleotides described herein are designed to caused RNase H mediated degradation of DMPK mRNA. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

Examples of oligonucleotides useful for targeting DMPK are provided in US Patent Application Publication 20100016215A1, published on Jan. 1, 2010, entitled Compound And Method For Treating Myotonic Dystrophy; US Patent Application Publication 20130237585A1, published Jul. 19, 2010, Modulation Of Dystrophia Myotonica-Protein Kinase (DMPK) Expression; US Patent Application Publication 20150064181A1, published on Mar. 5, 2015, entitled “Antisense Conjugates For Decreasing Expression Of Dmpk”; US Patent Application Publication 20150238627A1, published on Aug. 27, 2015, entitled “Peptide-Linked Morpholino Antisense Oligonucleotides For Treatment Of Myotonic Dystrophy”; and US Patent Application Publication 20160304877A1, published on Oct. 20, 2016, entitled “Compounds And Methods For Modulation Of Dystrophia Myotonica-Protein Kinase (Dmpk) Expression,” the contents of each of which are incorporated herein in their entireties.

In some embodiments, oligonucleotides may comprise a region of complementarity to a sequence set forth as follows, which is an example human DMPK gene sequence (Gene ID 1760; NM_001081560.2):

(SEQ ID NO: 24)
AGGGGGGCTGGACCAAGGGGTGGGGAGAAGGGGAGGAGGCCTCGGCCGGCCGCA
GAGAGAAGTGGCCAGAGAGGCCCAGGGGACAGCCAGGGACAGGCAGACATGCAG
CCAGGGCTCCAGGGCCTGGACAGGGGCTGCCAGGCCCTGTGACAGGAGGACCCCG
AGCCCCCGGCCCGGGGAGGGGCCATGGTGCTGCCTGTCCAACATGTCAGCCGAGG
TGCGGCTGAGGCGGCTCCAGCAGCTGGTGTTGGACCCGGGCTTCCTGGGGCTGGA
GCCCCTGCTCGACCTTCTCCTGGGCGTCCACCAGGAGCTGGGCGCCTCCGAACTGG
CCCAGGACAAGTACGTGGCCGACTTCTTGCAGTGGGCGGAGCCCATCGTGGTGAG
GCTTAAGGAGGTCCGACTGCAGAGGGACGACTTCGAGATTCTGAAGGTGATCGGA
CGCGGGGCGTTCAGCGAGGTAGCGGTAGTGAAGATGAAGCAGACGGGCCAGGTG
TATGCCATGAAGATCATGAACAAGTGGGACATGCTGAAGAGGGGCGAGGTGTCGT
GCTTCCGTGAGGAGAGGGACGTGTTGGTGAATGGGGACCGGCGGTGGATCACGCA
GCTGCACTTCGCCTTCCAGGATGAGAACTACCTGTACCTGGTCATGGAGTATTACG
TGGGCGGGGACCTGCTGACACTGCTGAGCAAGTTTGGGGAGCGGATTCCGGCCGA
GATGGCGCGCTTCTACCTGGCGGAGATTGTCATGGCCATAGACTCGGTGCACCGG
CTTGGCTACGTGCACAGGGACATCAAACCCGACAACATCCTGCTGGACCGCTGTG
GCCACATCCGCCTGGCCGACTTCGGCTCTTGCCTCAAGCTGCGGGCAGATGGAAC
GGTGCGGTCGCTGGTGGCTGTGGGCACCCCAGACTACCTGTCCCCCGAGATCCTGC
AGGCTGTGGGCGGTGGGCCTGGGACAGGCAGCTACGGGCCCGAGTGTGACTGGTG
GGCGCTGGGTGTATTCGCCTATGAAATGTTCTATGGGCAGACGCCCTTCTACGCGG
ATTCCACGGCGGAGACCTATGGCAAGATCGTCCACTACAAGGAGCACCTCTCTCT
GCCGCTGGTGGACGAAGGGGTCCCTGAGGAGGCTCGAGACTTCATTCAGCGGTTG
CTGTGTCCCCCGGAGACACGGCTGGGCCGGGGTGGAGCAGGCGACTTCCGGACAC
ATCCCTTCTTCTTTGGCCTCGACTGGGATGGTCTCCGGGACAGCGTGCCCCCCTTTA
CACCGGATTTCGAAGGTGCCACCGACACATGCAACTTCGACTTGGTGGAGGACGG
GCTCACTGCCATGGAGACACTGTCGGACATTCGGGAAGGTGCGCCGCTAGGGGTC
CACCTGCCTTTTGTGGGCTACTCCTACTCCTGCATGGCCCTCAGGGACAGTGAGGT
CCCAGGCCCCACACCCATGGAACTGGAGGCCGAGCAGCTGCTTGAGCCACACGTG
CAAGCGCCCAGCCTGGAGCCCTCGGTGTCCCCACAGGATGAAACAGCTGAAGTGG
CAGTTCCAGCGGCTGTCCCTGCGGCAGAGGCTGAGGCCGAGGTGACGCTGCGGGA
GCTCCAGGAAGCCCTGGAGGAGGAGGTGCTCACCCGGCAGAGCCTGAGCCGGGA
GATGGAGGCCATCCGCACGGACAACCAGAACTTCGCCAGTCAACTACGCGAGGCA
GAGGCTCGGAACCGGGACCTAGAGGCACACGTCCGGCAGTTGCAGGAGCGGATG
GAGTTGCTGCAGGCAGAGGGAGCCACAGCTGTCACGGGGGTCCCCAGTCCCCGGG
CCACGGATCCACCTTCCCATCTAGATGGCCCCCCGGCCGTGGCTGTGGGCCAGTGC
CCGCTGGTGGGGCCAGGCCCCATGCACCGCCGCCACCTGCTGCTCCCTGCCAGGGT
CCCTAGGCCTGGCCTATCGGAGGCGCTTTCCCTGCTCCTGTTCGCCGTTGTTCTGTC
TCGTGCCGCCGCCCTGGGCTGCATTGGGTTGGTGGCCCACGCCGGCCAACTCACCG
CAGTCTGGCGCCGCCCAGGAGCCGCCCGCGCTCCCTGAACCCTAGAACTGTCTTCG
ACTCCGGGGCCCCGTTGGAAGACTGAGTGCCCGGGGCACGGCACAGAAGCCGCGC
CCACCGCCTGCCAGTTCACAACCGCTCCGAGCGTGGGTCTCCGCCCAGCTCCAGTC
CTGTGATCCGGGCCCGCCCCCTAGCGGCCGGGGAGGGAGGGGCCGGGTCCGCGGC
CGGCGAACGGGGCTCGAAGGGTCCTTGTAGCCGGGAATGCTGCTGCTGCTGCTGC
TGCTGCTGCTGCTGCTGCTGCTGCTGCTGCTGCTGCTGCTGCTGGGGGGATCACAG
ACCATTTCTTTCTTTCGGCCAGGCTGAGGCCCTGACGTGGATGGGCAAACTGCAGG
CCTGGGAAGGCAGCAAGCCGGGCCGTCCGTGTTCCATCCTCCACGCACCCCCACCT
ATCGTTGGTTCGCAAAGTGCAAAGCTTTCTTGTGCATGACGCCCTGCTCTGGGGAG
CGTCTGGCGCGATCTCTGCCTGCTTACTCGGGAAATTTGCTTTTGCCAAACCCGCTT
TTTCGGGGATCCCGCGCCCCCCTCCTCACTTGCGCTGCTCTCGGAGCCCCAGCCGG
CTCCGCCCGCTTCGGCGGTTTGGATATTTATTGACCTCGTCCTCCGACTCGCTGACA
GGCTACAGGACCCCCAACAACCCCAATCCACGTTTTGGATGCACTGAGACCCCGA
CATTCCTCGGTATTTATTGTCTGTCCCCACCTAGGACCCCCACCCCCGACCCTCGCG
AATAAAAGGCCCTCCATCTGCCCAAAGCTCTGGA.

In some embodiments, oligonucleotides may comprise a region of complementarity to a sequence set forth as follows, which is an example mouse DMPK gene sequence (Gene ID 13400; NM_001190490.1).

(SEQ ID NO: 25)
GAACTGGCCAGAGAGACCCAAGGGATAGTCAGGGACGGGCAGACATGCAGCTAG
GGTTCTGGGGCCTGGACAGGGGCAGCCAGGCCCTGTGACGGGAAGACCCCGAGCT
CCGGCCCGGGGAGGGGCCATGGTGTTGCCTGCCCAACATGTCAGCCGAAGTGCGG
CTGAGGCAGCTCCAGCAGCTGGTGCTGGACCCAGGCTTCCTGGGACTGGAGCCCC
TGCTCGACCTTCTCCTGGGCGTCCACCAGGAGCTGGGTGCCTCTCACCTAGCCCAG
GACAAGTATGTGGCCGACTTCTTGCAGTGGGTGGAGCCCATTGCAGCAAGGCTTA
AGGAGGTCCGACTGCAGAGGGATGATTTTGAGATTTTGAAGGTGATCGGGCGTGG
GGCGTTCAGCGAGGTAGCGGTGGTGAAGATGAAACAGACGGGCCAAGTGTATGCC
ATGAAGATTATGAATAAGTGGGACATGCTGAAGAGAGGCGAGGTGTCGTGCTTCC
GGGAAGAAAGGGATGTATTAGTGAAAGGGGACCGGCGCTGGATCACACAGCTGC
ACTTTGCCTTCCAGGATGAGAACTACCTGTACCTGGTCATGGAATACTACGTGGGC
GGGGACCTGCTAACGCTGCTGAGCAAGTTTGGGGAGCGGATCCCCGCCGAGATGG
CTCGCTTCTACCTGGCCGAGATTGTCATGGCCATAGACTCCGTGCACCGGCTGGGC
TACGTGCACAGGGACATCAAACCAGATAACATTCTGCTGGACCGATGTGGGCACA
TTCGCCTGGCAGACTTCGGCTCCTGCCTCAAACTGCAGCCTGATGGAATGGTGAGG
TCGCTGGTGGCTGTGGGCACCCCGGACTACCTGTCTCCTGAGATTCTGCAGGCCGT
TGGTGGAGGGCCTGGGGCAGGCAGCTACGGGCCAGAGTGTGACTGGTGGGCACTG
GGCGTGTTCGCCTATGAGATGTTCTATGGGCAGACCCCCTTCTACGCGGACTCCAC
AGCCGAGACATATGCCAAGATTGTGCACTACAGGGAACACTTGTCGCTGCCGCTG
GCAGACACAGTTGTCCCCGAGGAAGCTCAGGACCTCATTCGTGGGCTGCTGTGTCC
TGCTGAGATAAGGCTAGGTCGAGGTGGGGCAGACTTCGAGGGTGCCACGGACACA
TGCAATTTCGATGTGGTGGAGGACCGGCTCACTGCCATGGTGAGCGGGGGCGGGG
AGACGCTGTCAGACATGCAGGAAGACATGCCCCTTGGGGTGCGCCTGCCCTTCGT
GGGCTACTCCTACTGCTGCATGGCCTTCAGAGACAATCAGGTCCCGGACCCCACCC
CTATGGAACTAGAGGCCCTGCAGTTGCCTGTGTCAGACTTGCAAGGGCTTGACTTG
CAGCCCCCAGTGTCCCCACCGGATCAAGTGGCTGAAGAGGCTGACCTAGTGGCTG
TCCCTGCCCCTGTGGCTGAGGCAGAGACCACGGTAACGCTGCAGCAGCTCCAGGA
AGCCCTGGAAGAAGAGGTTCTCACCCGGCAGAGCCTGAGCCGCGAGCTGGAGGCC
ATCCGGACCGCCAACCAGAACTTCTCCAGCCAACTACAGGAGGCCGAGGTCCGAA
ACCGAGACCTGGAGGCGCATGTTCGGCAGCTACAGGAACGGATGGAGATGCTGCA
GGCCCCAGGAGCCGCAGCCATCACGGGGGTCCCCAGTCCCCGGGCCACGGATCCA
CCTTCCCATCTAGATGGCCCCCCGGCCGTGGCTGTGGGCCAGTGCCCGCTGGTGGG
GCCAGGCCCCATGCACCGCCGTCACCTGCTGCTCCCTGCCAGGATCCCTAGGCCTG
GCCTATCCGAGGCGCGTTGCCTGCTCCTGTTCGCCGCTGCTCTGGCTGCTGCCGCC
ACACTGGGCTGCACTGGGTTGGTGGCCTATACCGGCGGTCTCACCCCAGTCTGGTG
TTTCCCGGGAGCCACCTTCGCCCCCTGAACCCTAAGACTCCAAGCCATCTTTCATT
TAGGCCTCCTAGGAAGGTCGAGCGACCAGGGAGCGACCCAAAGCGTCTCTGTGCC
CATCGCGCCCCCCCCCCCCCCCCACCGCTCCGCTCCACACTTCTGTGAGCCTGGGT
CCCCACCCAGCTCCGCTCCTGTGATCCAGGCCTGCCACCTGGCGGCCGGGGAGGG
AGGAACAGGGCTCGTGCCCAGCACCCCTGGTTCCTGCAGAGCTGGTAGCCACCGC
TGCTGCAGCAGCTGGGCATTCGCCGACCTTGCTTTACTCAGCCCCGACGTGGATGG
GCAAACTGCTCAGCTCATCCGATTTCACTTTTTCACTCTCCCAGCCATCAGTTACAA
GCCATAAGCATGAGCCCCCTATTTCCAGGGACATCCCATTCCCATAGTGATGGATC
AGCAAGACCTCTGCCAGCACACACGGAGTCTTTGGCTTCGGACAGCCTCACTCCTG
GGGGTTGCTGCAACTCCTTCCCCGTGTACACGTCTGCACTCTAACAACGGAGCCAC
AGCTGCACTCCCCCCTCCCCCAAAGCAGTGTGGGTATTTATTGATCTTGTTATCTG
ACTCACTGACAGACTCCGGGACCCACGTTTTAGATGCATTGAGACTCGACATTCCT
CGGTATTTATTGTCTGTCCCCACCTACGACCTCCACTCCCGACCCTTGCGAATAAA
ATACTTCTGGTCTGCCCTAAA.

In some embodiments, an oligonucleotide may comprise a region of complementarity to DMPK gene sequences of multiple species, e.g., selected from human, mouse and non-human species (e.g., cynomolgus monkey).

In some embodiments, the oligonucleotide may comprise a region of complementarity to a mutant form of DMPK, for example, a mutant form as reported in Botta A. et al. “The CTG repeat expansion size correlates with the splicing defects observed in muscles from myotonic dystrophy type 1 patients.” J Med Genet. 2008 October; 45(10):639-46; and Machuca-Tzili L. et al. “Clinical and molecular aspects of the myotonic dystrophies: a review.” Muscle Nerve. 2005 July; 32(1):1-18; the contents of each of which are incorporated herein by reference in their entireties.

In some embodiments, an oligonucleotide provided herein is an antisense oligonucleotide targeting DMPK. In some embodiments, the oligonucleotide targeting DMPK is any one of the antisense oligonucleotides targeting DMPK as described in US Patent Application Publication US20160304877A1, published on Oct. 20, 2016, entitled “Compounds And Methods For Modulation Of Dystrophia Myotonica-Protein Kinase (DMPK) Expression,” incorporated herein by reference). In some embodiments, the DMPK targeting oligonucleotide targets a region of the DMPK gene sequence as set forth in Genbank accession No. NM_001081560.2 (SEQ ID NO: 24) or as set forth in Genbank accession No. NG_009784.1 (SEQ ID NO: 26).

In some embodiments, a DMPK targeting oligonucleotide provided herein comprises a nucleotide sequence comprising a region complementary to a target region that is at least 8 continuous nucleotides (e.g., at least 8, at least 9, at least 10, at least 12, at least 14, at least 16, at least 18, at least 20 or more continuous nucleotides) of SEQ ID NO: 24.

In some embodiments, a DMPK targeting oligonucleotide provided herein is 10-35 (e.g., 10-35, 10-30, 10-25, 10-20, 10-15, 15-35, 15-30, 15-25, 15-20, 20-35, 20-30, 13-18, 14-17, 15-18, 20-30, 15-17, 27-30, 25-35, or 30-35) nucleotides in length. In some embodiments, a DMPK targeting oligonucleotide provided herein is 10, 11, 12, 13, 14, 15, 16, 17, 18, 19, 20, 21, 22, 23, 24, 25, 26, 27, 28, 29, or 30 nucleotides in length, optionally 15-30, or 16 nucleotides in length. In some embodiments, a DMPK targeting oligonucleotide provided herein is 16 nucleotides in length.

In some embodiments, a DMPK targeting oligonucleotide provided herein comprises a region of complementarity of at least 8 (e.g., at least 8, 9, 10, 11, 12, 13, 14, 15, 16, 17, 18, 19, 20, 21, 22, 23, 24, 25, 26, 27, 28, 29, 30, or more) consecutive nucleotides to a DMPK RNA.

In some embodiments, a DMPK targeting oligonucleotide provided herein comprises a region of complementarity of at least 8 (e.g., at least 8, 9, 10, 11, 12, 13, 14, 15, 16, 17, 18, 19, 20, 21, 22, 23, 24, 25, 26, 27, 28, 29, 30, or more) consecutive nucleotides to a DMPK sequence as set forth in SEQ ID NO: 24 or 25.

In some embodiments, a DMPK targeting oligonucleotide provided herein comprises a region of complementarity of at least 8 (e.g., at least 8, 9, 10, 11, 12, 13, 14, 15, or 16) consecutive nucleotides to a target sequence as set forth in SEQ ID NO: 22 (TGACTGGTGGGCGCTG). In some embodiments, an oligonucleotide useful for targeting DMPK comprises at least 8 (e.g., at least 8, 9, 10, 11, 12, 13, 14, 15, or 16) consecutive nucleotides of a sequence as set forth in SEQ ID NO: 21 (CAGCGCCCACCAGUCA). In some embodiments, an oligonucleotide useful for targeting DMPK comprises the nucleotide sequence of SEQ ID NO: 21.

In some embodiments, the DMPK targeting oligonucleotide comprises a 5′-X—Y—Z-3′ configuration. An oligonucleotide comprising a 5′-X—Y—Z-3′ configuration can refer to a chimeric antisense compound in which a gap region having a plurality of nucleosides that support RNase H cleavage is positioned between flanking regions having one or more nucleosides, wherein the nucleosides comprising the gap region are chemically distinct from the nucleoside or nucleosides comprising the flanking regions. In some embodiments, an oligonucleotide described herein (e.g., a DMPK-targeting oligonucleotide described herein) comprises a 5′-X—Y—Z-3′ configuration, with X and Z as flanking regions around a gap region Y. In some embodiments, the gap region Y comprises one or more 2′-deoxyribonucleosides. In some embodiments, each nucleoside in the gap region Y is a 2′-deoxyribonucleoside, and neither the flanking region X nor the flanking region Z contains any 2′-deoxyribonucleosides.

In some embodiments, the gap region Y comprises a continuous stretch of 6 or more 2′-deoxyribonucleosides, which are capable of recruiting an RNAse, such as RNAse H. In some embodiments, the oligonucleotide binds to the target nucleic acid, at which point an RNAse is recruited and can then cleave the target nucleic acid. In some embodiments, the flanking regions X and Z each comprise one or more modified nucleosides. In some embodiments, flanking regions X and Z each comprise one or more high-affinity modified nucleosides, e.g., one to six high-affinity modified nucleosides. Examples of high affinity modified nucleosides include, but are not limited to, 2′-modified nucleosides (e.g., 2′-MOE, 2′-O-Me, 2′-F) or 2′-4′ bicyclic nucleosides (e.g., LNA, cEt, ENA). In some embodiments, the flanking regions X and Z may be of 1-20 nucleotides, 1-8 nucleotides, or 1-5 nucleotides in length. The flanking regions X and Z may be of similar length or of dissimilar lengths. In some embodiments, the gap region Y may comprise a nucleotide sequence of 5-20 nucleotides, 5-15 nucleotides, 5-12 nucleotides, or 6-10 nucleotides in length.

In some embodiments, the gap region Y comprises one or more unmodified internucleoside linkages. In some embodiments, one or both flanking regions X and Z each independently comprise phosphorothioate internucleoside linkages (e.g., phosphorothioate internucleoside linkages or other linkages) between at least two, at least three, at least four, at least five or more nucleotides. In some embodiments, the gap region Y and two flanking regions X and Z each independently comprise modified internucleoside linkages (e.g., phosphorothioate internucleoside linkages or other linkages) between at least two, at least three, at least four, at least five or more nucleotides.

In some embodiments, the gap region Y in the gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosine.

In some embodiments, the flanking region X of the oligonucleotide (X in the 5′-X—Y—Z-3′ configuration) and the flanking region Z of the oligonucleotide (Z in the 5′-X—Y—Z-3′ configuration) are independently 1-20 nucleosides long. For example, the flanking region X of the oligonucleotide and the flanking region Z of the oligonucleotide may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the flanking region X of the oligonucleotide and the flanking region Z of the oligonucleotide are independently 1, 2, 3, 4, 5, 6, 7, 8, 9, 10, 11, 12, 13, 14, 15, 16, 17, 18, 19, or 20 nucleosides long. In some embodiments, the flanking region X of the oligonucleotide and the flanking region Z of the oligonucleotide are of the same length. In some embodiments, the flanking region X of the oligonucleotide and the flanking region Z of the oligonucleotide are of different lengths. In some embodiments, the flanking region X of the oligonucleotide is longer than the flanking region Z of the oligonucleotide. In some embodiments, the flanking region X of the oligonucleotide is shorter than the flanking region Z of the oligonucleotide.

In some embodiments, an oligonucleotide described herein (e.g., a DMPK targeting oligonucleotide) comprises a 5′-X—Y—Z-3′ configuration of 5-10-5, 4-12-4, 3-14-3, 2-16-2, 1-18-1, 3-3, 2-10-2, 1-10-1, 2-8-2, 4-6-4, 3-6-3, 2-6-2, 4-7-4, 3-7-3, 2-7-2, 4-8- 4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12-3, 4-12- 7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions, respectively, in an oligonucleotide comprising the 5′-X—Y—Z-3′ configuration.

In some embodiments, one or more nucleosides in the flanking region X of the oligonucleotide (X in the 5′-X—Y—Z-3′ configuration) or the flanking region Z of the oligonucleotide (Z in the 5′-X—Y—Z-3′ configuration) are modified nucleosides (e.g., high-affinity modified nucleosides). In some embodiments, the modified nucleoside (e.g., high-affinity modified nucleosides) is a 2′-modified nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA)).

In some embodiments, an oligonucleotide described herein (e.g., a DMPK targeting oligonucleotide described herein) comprises a 5′-X—Y—Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′ deoxyribonucleoside. In some embodiments, an oligonucleotide described herein (e.g., a DMPK targeting oligonucleotide described herein) comprises a 5′-X—Y—Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′ deoxyribonucleoside. In some embodiments, an oligonucleotide described herein (e.g., a DMPK targeting oligonucleotide described herein) comprises a 5′-X—Y—Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′ deoxyribonucleoside.

In some embodiments, an oligonucleotide described herein (e.g., a DMPK targeting oligonucleotide) is 10⁻²⁰ nucleosides (e.g., 10, 11, 12, 13, 14, 15, 16, 17, 18, 19, or 20 nucleosides) in length, comprises a region of complementarity to at least 8 consecutive nucleosides (e.g., at least 8, at least 9, at least 10, at least 11, at least 12, at least 13, at least 14, at least 15, or 16 consecutive nucleosides) of SEQ ID NO: 22 (TGACTGGTGGGCGCTG), and comprises a 5′-X—Y—Z-3′ configuration, wherein X comprises 3-5 (e.g., 3, 4, or 5) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-10 (e.g., 6, 7, 8, 9, or 10) linked 2′-deoxyribonucleosides, wherein each cytosine in Y is optionally and independently a 5-methyl-cytosine; and Z comprises 3-5 (e.g., 3, 4, or 5) linked nucleosides, wherein at least one of the nucleosides in Z is a 2′-modified nucleoside (e.g., 2′-MOE modified nucleoside, 2′-O-Me modified nucleoside, LNA, cEt, or ENA).

In some embodiments, an oligonucleotide described herein (e.g., a DMPK targeting oligonucleotide described herein) comprises at least 8 consecutive nucleosides (e.g., at least 8, at least 9, at least 10, at least 11, at least 12, at least 13, at least 14, at least 15, or 16 consecutive nucleosides consecutive nucleosides) of the nucleotide sequence of SEQ ID NO: 21 (CAGCGCCCACCAGUCA), and comprises a 5′-X—Y—Z-3′ configuration, wherein X comprises 3-5 (e.g., 3, 4, or 5) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-10 (e.g., 6, 7, 8, 9, or 10) linked 2′-deoxyribonucleosides, wherein each cytosine in Y is optionally and independently a 5-methyl-cytosine; and Z comprises 3-5 (e.g., 3, 4, or 5) linked nucleosides, wherein at least one of the nucleosides in Z is a 2′-modified nucleoside (e.g., 2′-MOE modified nucleoside, 2′-O-Me modified nucleoside, LNA, cEt, or ENA). In some embodiments, each thymine base (T) of the nucleotide sequence of the antisense oligonucleotide may independently and optionally be replaced with a uracil base (U), and each U may independently and optionally be replaced with a T.

In some embodiments, an oligonucleotide described herein (e.g., a DMPK targeting oligonucleotide described herein) comprises the nucleotide sequence of SEQ ID NO: 21 and comprises a 5′-X—Y—Z-3′ configuration, wherein X comprises 3-5 (e.g., 3, 4, or 5) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-10 (e.g., 6, 7, 8, 9, or 10) linked 2′-deoxyribonucleosides, wherein each cytosine in Y is optionally and independently a 5-methyl-cytosine; and Z comprises 3-5 (e.g., 3, 4, or 5) linked nucleosides, wherein at least one of the nucleosides in Z is a 2′-modified nucleoside (e.g., 2′-MOE modified nucleoside, 2′-O-Me modified nucleoside, LNA, cEt, or ENA). In some embodiments, each thymine base (T) of the nucleotide sequence of the antisense oligonucleotide may independently and optionally be replaced with a uracil base (U), and each U may independently and optionally be replaced with a T.

In some embodiments, X comprises at least one 2′-4′ bicyclic nucleoside (e.g., LNA, cEt, or ENA) and at least one non-bicyclic 2′-modified nucleoside e.g., 2′-MOE modified nucleoside or 2′-O-Me modified nucleoside, and/or (e.g., and) Z comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside).

In some embodiments, the 2′-4′ bicyclic nucleoside is selected from LNA, cEt, and ENA nucleosides. In some embodiments, the non-bicyclic 2′-modified nucleoside is a 2′-MOE modified nucleoside or a 2′-OMe modified nucleoside.

In some embodiments, the nucleosides of the oligonucleotides are joined together by phosphorothioate internucleoside linkages, phosphodiester internucleoside linkages or a combination thereof. In some embodiments, the oligonucleotide comprises only phosphorothioate internucleoside linkages joining each nucleoside (i.e., the oligonucleotide comprises a fully phosphorothioate backbone). In some embodiments, the oligonucleotide comprises at least one phosphorothioate internucleoside linkage. In some embodiments, the oligonucleotide comprises a mix of phosphorothioate and phosphodiester internucleoside linkages. In some embodiments, the oligonucleotide comprises only phosphorothioate internucleoside linkages joining each pair of 2′-deoxyribonucleosides and a mix of phosphorothioate and phosphodiester internucleoside linkages joining the remaining nucleosides.

In some embodiments, the oligonucleotide comprises a 5′-X—Y—Z-3′ configuration of LLEE-(D)₈-EELL, wherein “E” is a 2′-MOE modified ribonucleoside; “L” is LNA; “D” is a 2′-deoxyribonucleoside; and “10” or “8” is the number of 2′-deoxyribonucleosides in Y, and wherein the oligonucleotide comprises phosphorothioate internucleoside linkages, phosphodiester internucleoside linkages or a combination thereof.

In some embodiments, each cytidine (e.g., a 2′-modified cytidine) in X and/or Z of the oligonucleotide is optionally and independently a 5-methyl-cytidine, and/or each uridine (e.g., a 2′-modified uridine) in X and/or Z is optionally and independently a 5-methyl-uridine.

In some embodiments, an oligonucleotide described herein (e.g., a DMPK targeting oligonucleotide described herein) comprises a 5′-X—Y—Z-3′ configuration and comprises a nucleobase sequence of CAGCGCCCACCAGUCA (SEQ ID NO: 21). In some embodiments, an oligonucleotide described herein (e.g., a DMPK targeting oligonucleotide described herein) comprises a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage.

In some embodiments, an oligonucleotide described herein (e.g., a DMPK targeting oligonucleotide described herein) comprises a structure of the formula (Ie):

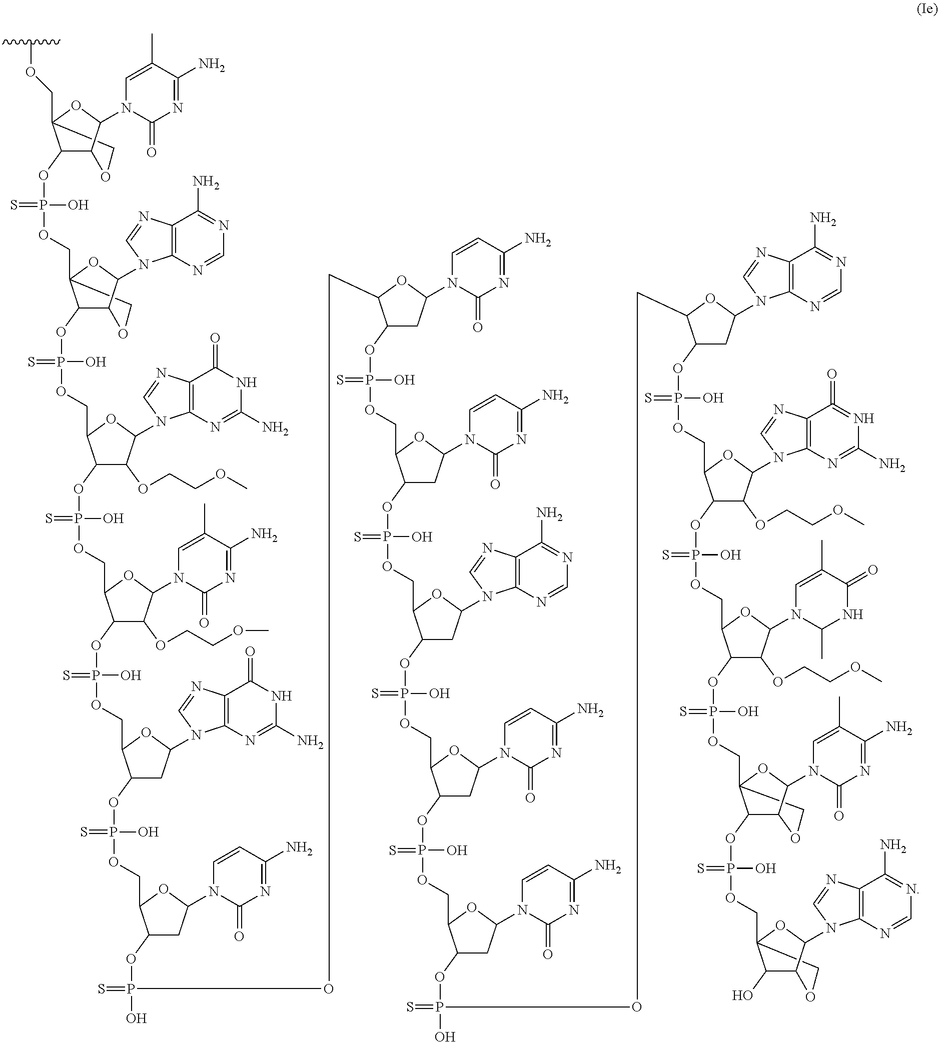

In some embodiments, an oligonucleotide described herein (e.g., a DMPK targeting oligonucleotide described herein) can be in salt form, e.g., as sodium, potassium, or magnesium salts.

In some embodiments, the 5′ or 3′ nucleoside (e.g., terminal nucleoside) of the oligonucleotide is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of an oligonucleotide described herein is covalently linked to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—. —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of the oligonucleotide is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via an amine group of a lysine of the targeting agent.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein may independently and optionally be uracil bases (U's), and/or any one or more of the U's in the oligonucleotides provided herein (e.g., the oligonucleotide as set forth in SEQ ID NO: 21) may independently and optionally be T's.

### Compositions

In some embodiments, compositions comprising complexes (i.e., a plurality of complexes) are prepared in a manner suitable for the methods described herein. In some embodiments, compositions comprising muscle-targeting complexes are delivered to a subject in a manner that minimizes degradation, facilitates delivery and/or (e.g., and) uptake, or provides another beneficial property to the complexes in the composition. Accordingly, in some embodiments, compositions comprising complexes (e.g., a plurality of complexes comprising an oligonucleotide covalently linked with a Fab) comprise tris(hydroxymethyl)aminomethane and/or sucrose. In some embodiments, compositions comprising muscle-targeting complexes (e.g., complexes comprising an oligonucleotide covalently linked with a Fab) comprise tris(hydroxymethyl)aminomethane and/or sucrose in aqueous solutions. In some embodiments, compositions comprising a plurality of the complexes, tris(hydroxymethyl)aminomethane, and sucrose can be lyophilized (e.g., for storage). In some embodiments, the lyophilized composition may be reconstituted (e.g., with water) for administration to a subject. In some embodiments, compositions comprising a plurality of the complexes, tris(hydroxymethyl)aminomethane, and sucrose can be frozen (e.g., for storage). In some embodiments, the frozen composition may be thawed prior to administration to a subject, e.g., to produce an aqueous solution. The compositions (e.g., in aqueous solutions, in frozen compositions, or in lyophilized compositions) can be suitably prepared such that when administered to a subject, either into the immediate environment of a target cell or systemically, a sufficient amount of the complexes enter target muscle cells.

In some embodiments, compositions (e.g., in aqueous solutions) for administration to a subject in the methods described herein comprise complexes (i.e., a plurality of complexes), each of which complex comprises an oligonucleotide (e.g., an oligonucleotide comprising a 5′-X—Y—Z-3′ configuration) covalently linked to an antibody. In some embodiments, compositions (e.g., in aqueous solutions) for administration to a subject in the methods described herein comprise complexes, in which each complex comprises an oligonucleotide (e.g., an oligonucleotide comprising a 5′-X—Y—Z-3′ configuration) covalently linked to an anti-TfR1 antibody, optionally wherein the antibody of such complexes comprises a CDR-H1, CDR-H2, CDR-H3, CDR-L1, CDR-L2, and CDR-L3 as set forth in Table 2, and further, in some embodiments, wherein the composition further comprises tris(hydroxymethyl)aminomethane and sucrose. In some embodiments, the antibody is an anti-TfR1 Fab.

In some embodiments, compositions (e.g., in aqueous solutions) for administration to a subject in the methods described herein comprise complexes (i.e., a plurality of complexes) comprising a structure of formula (I): [R¹]ₙ₁—R², in which each R¹ independently comprises a compound comprising an oligonucleotide (e.g., an oligonucleotide comprising a 5′-X—Y—Z-3′ configuration) and is covalently linked to R², wherein R² comprises an antibody (e.g., anti-TfR1 antibody), and in which in each complex n1 is independently an integer of one or greater representing the number of instances of R¹ in each complex.

In some embodiments, the value of n1 of each complex in the composition is independently and optionally an integer from one up to the number of amino acid residues to which conjugation is desired or targeted (e.g., the number of lysine residues) in the antibody (R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 2, 1 to 3, 1 to 5, 1 to 10, 1 to 26, or 1 to 27.

In some embodiments, a composition for administration to a subject in the methods described herein comprises unconjugated antibody (e.g., in trace amounts) and antibody conjugated to one or more oligonucleotides. In some embodiments, unconjugated antibody may be referred to as a compound comprising a structure of formula (I): [R¹]ₙ₁—R², for which n1 is zero. Accordingly, in some embodiments, a composition for administration to a subject in the methods described herein comprises compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, each instance of R¹ in a complex herein (e.g., a complex of a composition provided herein) is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, compositions are provided in which complexes for which R¹ is covalently linked to an amino acid residue residing in a CDR region of the antibody are present in only trace amounts, or in undetectable amount, or not at all. In some embodiments, compositions are provided in which complexes for which R¹ is covalently linked to an amino acid residue residing in a CDR region of the antibody are not detectable in the composition using standard detection techniques.

In some embodiments, compositions (e.g., in aqueous solutions) for administration to a subject in the methods described herein comprise complexes that comprise a structure of formula (I): [R¹]ₙ₁—R², in which each instance of R¹ in a complex of a composition provided herein independently comprises a group of the formula (Ia):

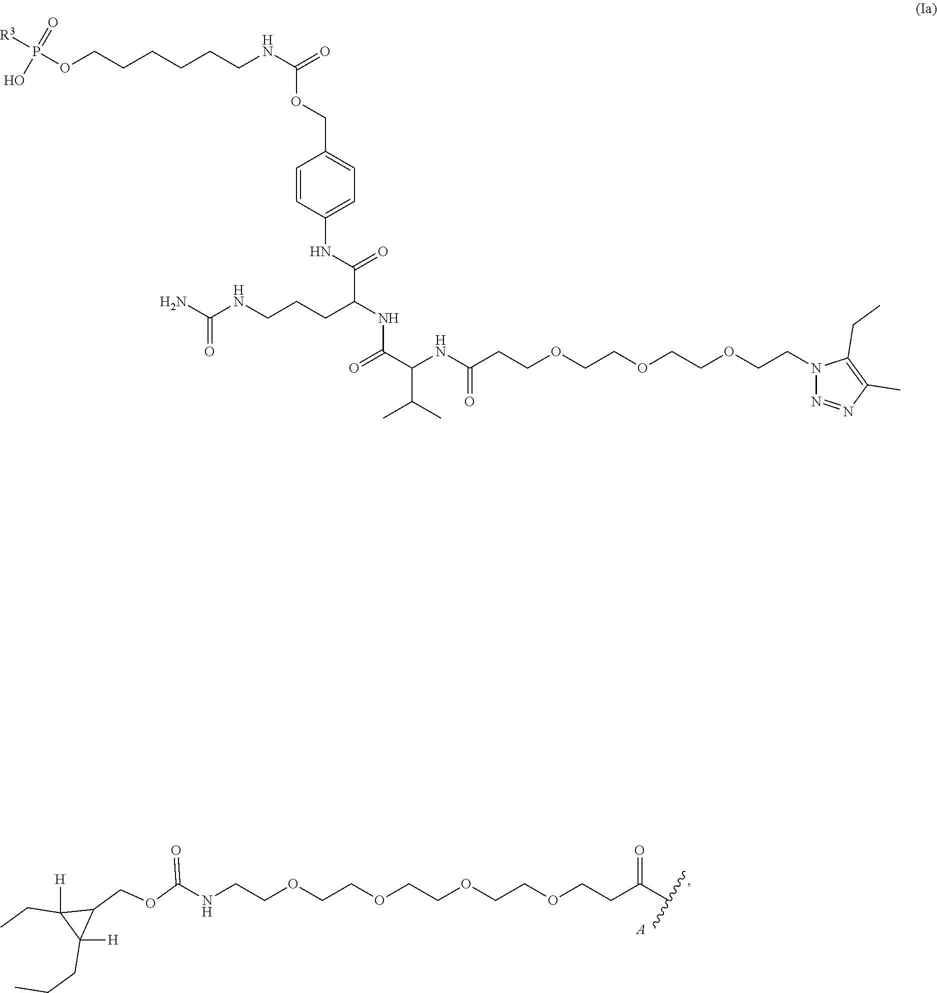

in which R³ comprises an oligonucleotide, e.g., an oligonucleotide comprising a 5′-X—Y—Z-3′ configuration; and R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R³ comprises an oligonucleotide comprising a nucleobase sequence of CAGCGCCCACCAGUCA (SEQ ID NO: 21). In some embodiments, R³ comprises an oligonucleotide comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage. In some embodiments, in each complex n1 is independently an integer (e.g., an integer in the range of 1-27, 1-26, 1-10, 1-5, or 1-3). In some embodiments, compositions (e.g., in aqueous solutions) for administration to a subject in the methods described herein further comprise complexes in which n1 is 0.

In some embodiments, compositions (e.g., in aqueous solutions) for administration to a subject in the methods described herein comprise complexes that comprise a structure of formula (I): [R¹]ₙ₁—R², in which each instance of R¹ in a complex of a composition provided herein comprises a group of the formula (Ib):

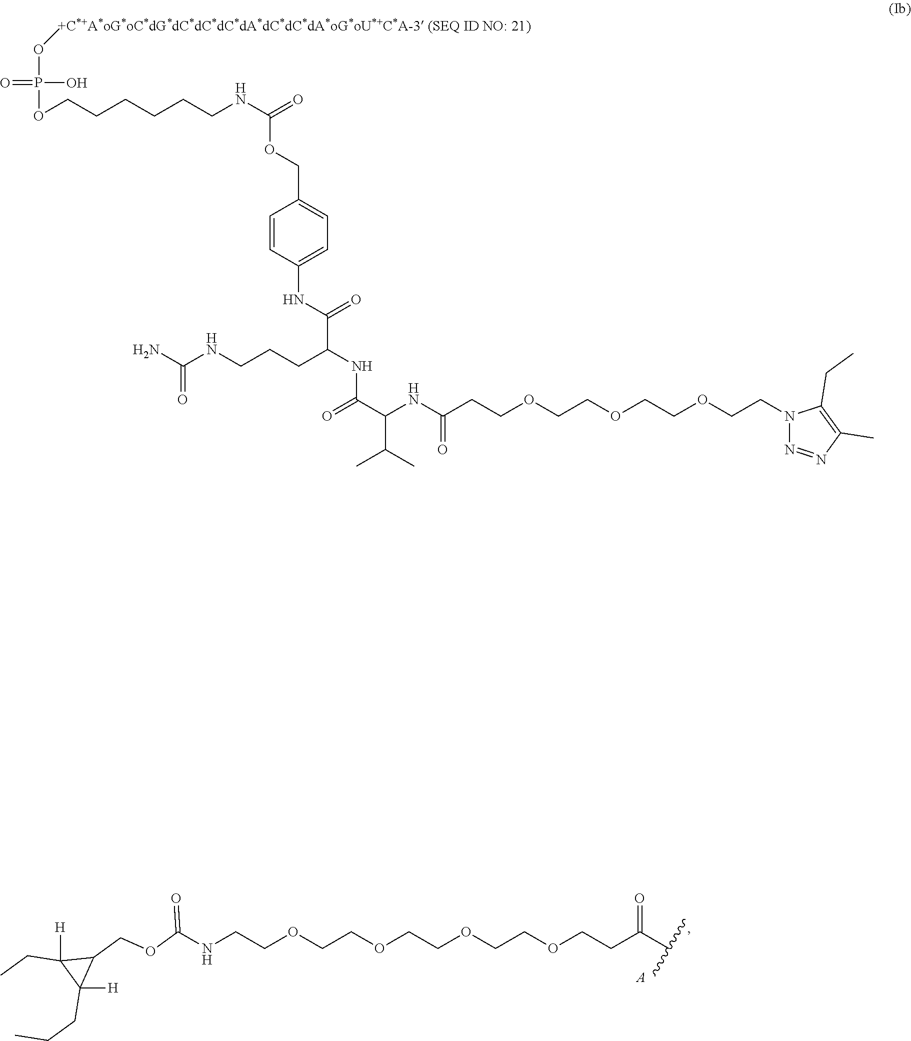

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21), wherein n1 is an integer (e.g., one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, compositions (e.g., in aqueous solutions) for administration to a subject in the methods described herein further comprise complexes in which n1 is 0.

In some embodiments, compositions (e.g., in aqueous solutions) for administration to a subject in the methods described herein comprise complexes that comprise a structure of formula (I): [R¹]ₙ₁—R², in which each instance of R¹ in a complex of a composition provided herein comprises a group of the formula (Ic):

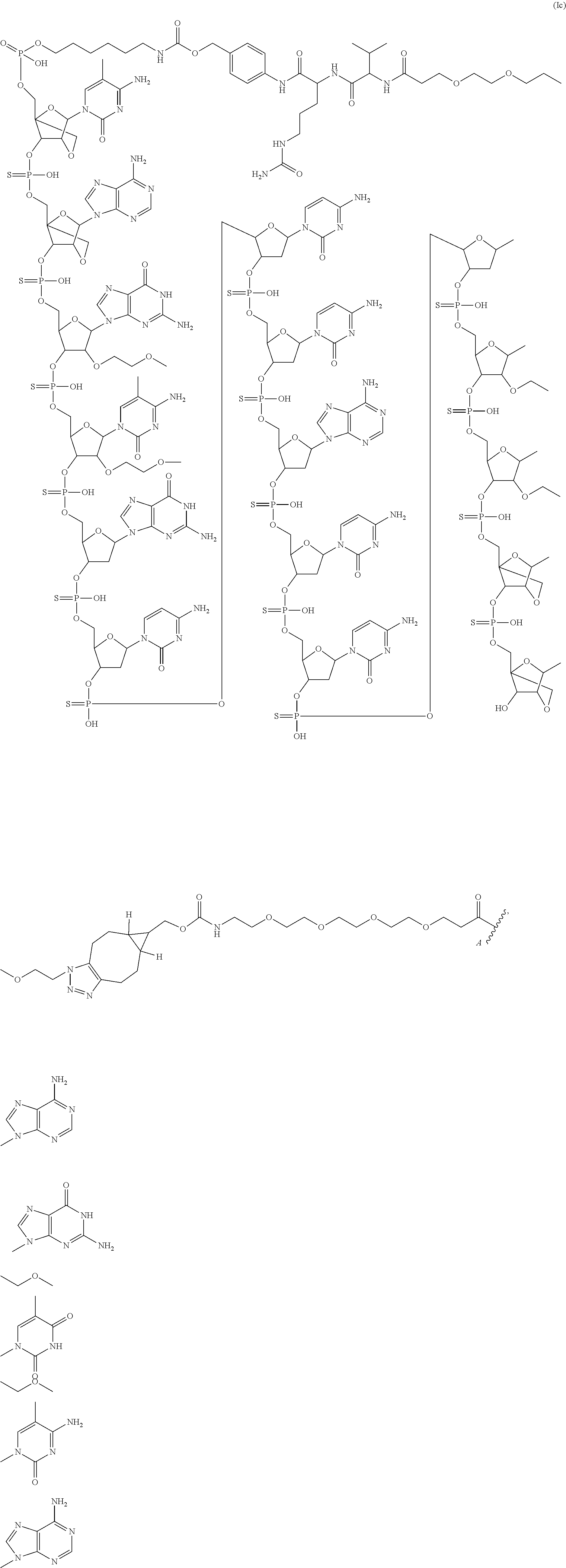

wherein R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, compositions (e.g., in aqueous solutions) for administration to a subject in the methods described herein further comprise complexes in which n1 is 0.

In some embodiments, compositions (e.g., in aqueous solutions) for administration to a subject in the methods described herein comprise complexes that comprise a structure of the formula (Id):

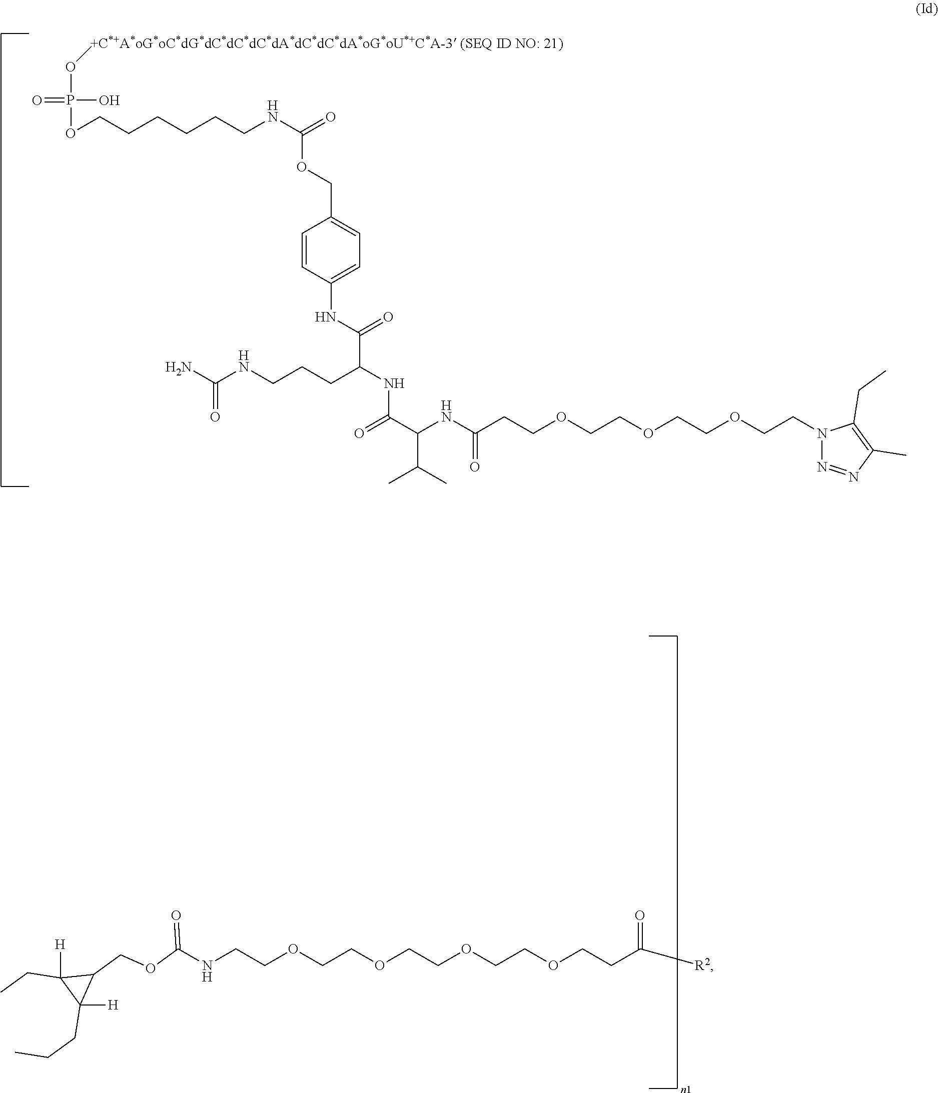

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21); wherein R² comprises an antibody comprising a sequence as set forth in Table 2; wherein n1 is an integer (e.g., one or greater) representing the number of instances of the group enclosed by square brackets, wherein each instance of the group enclosed by square brackets is covalently linked to a different amino acid residue of the antibody, optionally wherein each different amino acid residue is a lysine. In some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, compositions (e.g., in aqueous solutions) for administration to a subject in the methods described herein further comprise complexes in which n1 is 0.

In some embodiments, compositions (e.g., aqueous solutions) for administration to a subject in the methods described herein comprise a structure of formula (A):

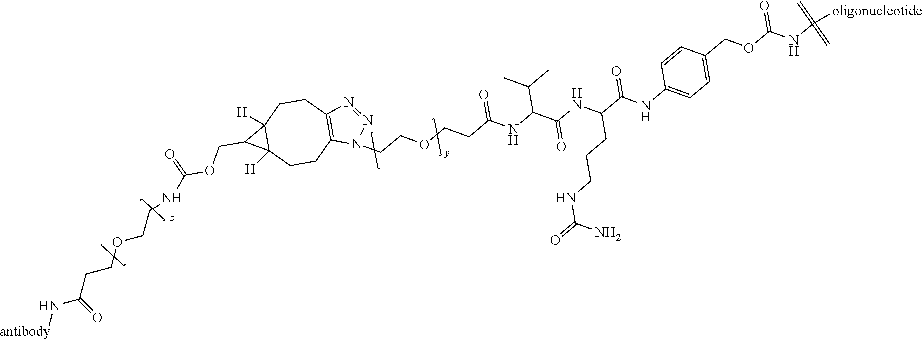

(A), wherein y is 0-15 (e.g., 3) and z is 0-15 (e.g., 4). In some embodiments, the amide shown adjacent the antibody (e.g., anti-TfR1 antibody) in the structure (A) results from a reaction with an amine of the antibody, such as a lysine epsilon amine. In some embodiments, a complex described herein comprises an anti-TfR1 antibody (e.g., an anti-TfR1 Fab) covalently linked via a lysine of the antibody to the 5′ end of an oligonucleotide (e.g., an oligonucleotide comprising a 5′-X—Y—Z-3′ configuration). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment.

In some embodiments, a composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment/Dosing

Complexes comprising an anti-TfR1 antibody (e.g., Fab) covalently linked to a molecular payload (e.g., a DMPK-targeting oligonucleotide) as described herein are effective in treating a subject having a myotonic dystrophy, e.g., DM1. In some embodiments, complexes comprise a molecular payload that is an oligonucleotide, e.g., an oligonucleotide that facilitates reduced expression or activity of DMPK (e.g., reduced level of a mutant or wild-type DMPK RNA) in a subject (e.g., a subject having DM1).

In some embodiments, a subject may be a human subject, a non-human primate subject (e.g., cynomolgus monkey), a rodent subject, or any suitable mammalian subject. In some embodiments, the subject is human. In some embodiments, the subject is a neonatal or young pediatric human subject (e.g., a human subject that is less than 4 years old, less than 3 years old, less than 2 years old, less than 1 year old, about 11 months old, about 10 months old, about 9 months old, about 8 months old, about 7 months old, about 6 months old, about 5 months old, about 4 months old, about 3 months old, about 2 months old, or about 1 month old). In some embodiments, the subject is a human subject that is between 2 and 60 (e.g., 2-60, 2-50, 2-40, 2-30, 2-20, 2-10) years old. In some embodiments, the subject is a human subject that is between 5 and 30 (e.g., 5, 6, 7, 8, 9, 10, 11, 12, 13, 14, 15, 16, 17, 18, 19, 20, 21, 22, 23, 24, 25, 26, 27, 28, 29, or 30) years old. In some embodiments, the subject is a human subject that is between 18 and 50 (e.g., 18-50, 18-45, 18-40, 18-35, 18-30, 18-25, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) years old. In some embodiments, the subject is a human subject that is 60 years old or older (e.g., about 60 years old, about 65 years old, about 70 years old, about 75 years old, or about 80 years old). In some embodiments, the subject is a human subject that is between 5 and 12 (e.g., 5, 6, 7, 8, 9, 10, 11, or 12) years old. In some embodiments, the subject is a human subject that is 4-16 (e.g., 4-16, 5-16, 6-16, 7-16, 8-16, 9-16, 10-16, 11-16, 12-16, 13-16, 14-16, 15-16, 4-15, 5-15, 6-15, 7-15, 8-15, 9-15, 10-15, 11-15, 12-15, 13-15, 14-15, 4-14, 5-14, 6-14, 7-14, 8-14, 9-14, 10-14, 11-14, 12-14, 13-14, 4-13, 5-13, 6-13, 7-13, 8-13, 9-13, 10-13, 11-13, 12-13, 4-12, 5-12, 6-12, 7-12, 8-12, 9-12, 10-12, 11-12, 4-11, 5-11, 6-11,7-11, 8-11, 9-16, 10-11, 4-10, 5-10, 6-10, 7-10, 8-10, 9-10, 4-9, 5-9, 6-9, 7-9, 8-9, 4-9, 5-9, 6-9, 7-9, 8-9, 4-8, 5-8, 6-8, 7-8, 4-7, 5-7, 6-7, 4-6, 5-6, or 4-5) years old. In some embodiments, the subject is a human subject that is about 4, 5, 6, 7, 8, 9, 10, 11, 12, 13, 14, 15, or 16 years old.

In some embodiments, a subject may have myotonic dystrophy, such as DM1. In some embodiments, a subject has a DMPK allele, which may optionally contain a disease-associated repeat, e.g., a CTG trinucleotide repeat expansion. In some embodiments, a subject may have a DMPK allele with an expanded disease-associated-repeat that comprises about 2-10 repeat units, about 2-50 repeat units, about 2-100 repeat units, about 50-1,000 repeat units, about 50-500 repeat units, about 50-250 repeat units, about 50-100 repeat units, about 500-10,000 repeat units, about 500-5,000 repeat units, about 500-2,500 repeat units, about 500-1,000 repeat units, or about 1,000-10,000 repeat units. In some embodiments, a subject is suffering from symptoms of DM1, e.g. muscle atrophy, muscle loss, excessive daytime sleepiness or cognitive delay. In some embodiments, a subject is not suffering from symptoms of DM1. In some embodiments, subjects have congenital myotonic dystrophy. In some embodiments, a subject is ambulant. In some embodiments, a subject is non-ambulant.

An aspect of the disclosure includes methods involving administering to a subject an effective amount of a composition (e.g., aqueous solution) comprising complexes as described herein. In some embodiments, an effective amount of a composition (e.g., aqueous solution) that comprises complexes comprising an anti-TfR1 antibody (e.g., Fab) described herein covalently linked to an oligonucleotide (e.g., a DMPK-targeting oligonucleotide) described herein can be administered to a subject in need of treatment. In some embodiments, a composition (e.g., aqueous solution) is administered systemically. In some embodiments, a pharmaceutical composition comprising complexes as described herein may be administered by a suitable route, which may include intravenous administration, e.g., as a bolus or by continuous infusion over a period of time. In some embodiments, administration may be performed by intravenous, intramuscular, intraperitoneal, intracerebrospinal, subcutaneous, intra-articular, intrasynovial, or intrathecal routes. In some embodiments, a composition (e.g., aqueous solution) comprising complexes as described herein is administered by infusion (e.g., intravenous infusion).

In some embodiments, a composition comprising a plurality of complexes described herein may be in solid form, aqueous form, or a liquid form. In some embodiments, an aqueous or liquid form may be nebulized or lyophilized. In some embodiments, a lyophilized form may be reconstituted with an aqueous or liquid solution (e.g., prior to administration, such as by intravenous infusion).

In some embodiments, provided are methods of and/or uses for treating a subject having a DMPK allele associated with myotonic dystrophy (e.g., DM1), comprising administering to the subject a composition comprising a complex or plurality of complexes described herein with an effective amount of the complexes. In some embodiments, provided are methods of and/or uses for reducing the expression or activity of DMPK (e.g., reducing the level of a mutant or wild-type DMPK RNA, or the activity of a DMPK gene product) in a subject, the methods comprising contacting the cell with the composition comprising a plurality of complexes described herein with an effective amount of the complex(es). In some embodiments, the method comprises administering a lyophilized form (e.g., lyophilized powder) of a composition comprising a plurality of complexes described herein, comprising reconstituting a lyophilized form of the composition in an aqueous solution, and administering the aqueous solution to a subject in need thereof. For example, in some embodiments, a lyophilized form of the composition comprising a complex or plurality of complexes is shipped and/or stored in the lyophilized form, reconstituted in an aqueous solution at a location (e.g., healthcare provider location) for administration, and administered in the reconstituted form (e.g., as an aqueous solution) by injection or intravenously, e.g., by infusion. In some embodiments, the subject has a DMPK allele, which may optionally contain a disease-associated repeat, e.g., a CTG trinucleotide repeat expansion.

In some embodiments, a composition is administered via site-specific or local delivery techniques. Examples of these techniques include implantable depot sources of the complex, local delivery catheters, site specific carriers, direct injection, or direct application.

In some aspects, provided herein are methods of reducing expression or activity of DMPK (e.g., reducing the level of a mutant or wild-type DMPK RNA, or the activity of a DMPK gene product) in a subject. In some aspects, provided herein are methods of treating myotonic dystrophy (e.g., DM1) in a subject. In some embodiments, methods provided herein comprise administering to the subject a composition comprising an effective amount of complexes, each complex comprising an anti-transferrin receptor 1 (TfR1) antibody (e.g., a Fab) covalently linked to one or more oligonucleotides, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises a nucleobase sequence of CAGCGCCCACCAGUCA (SEQ ID NO: 21),
optionally wherein the oligonucleotide comprises a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
further optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20.

In some embodiments, methods provided herein comprise administering to the subject a composition comprising an effective amount of complexes, wherein each complex comprises a structure of formula (I): [R¹]ₙ₁—R², wherein each instance of R¹ in a complex of a composition provided herein comprises a group of the formula (Ia):

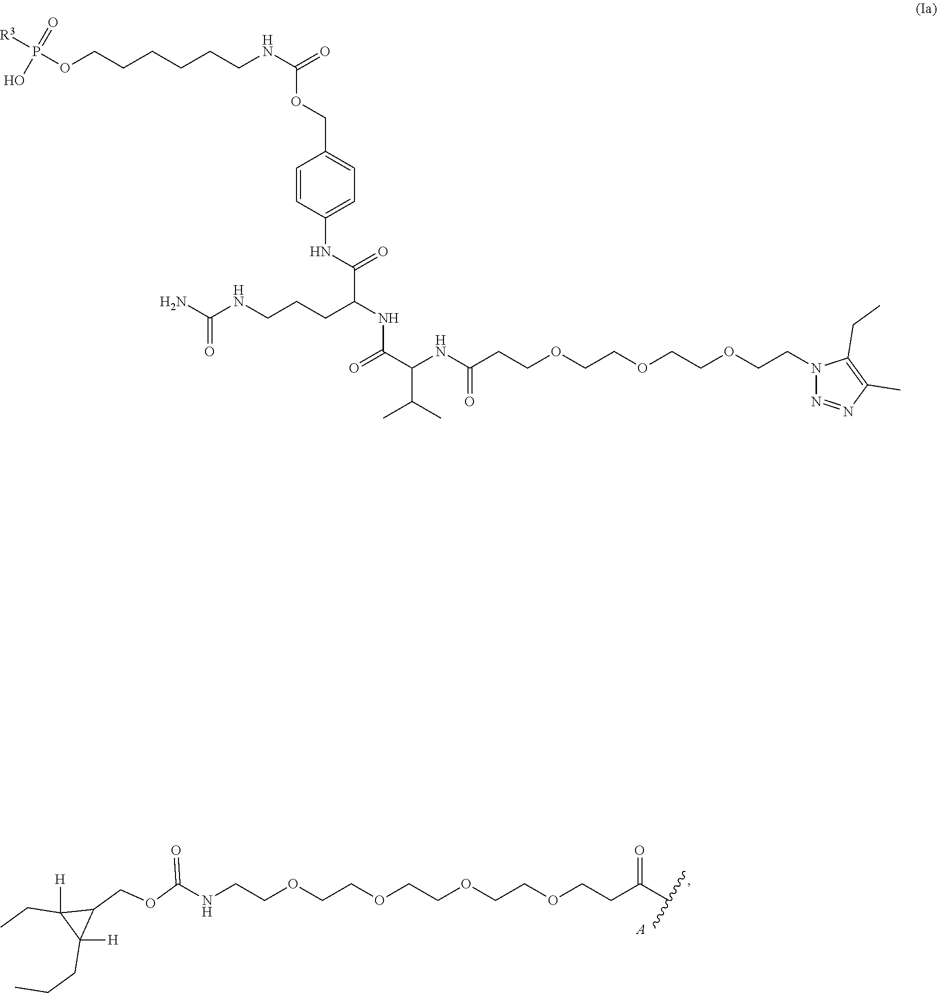

in which R³ comprises an oligonucleotide comprising a nucleobase sequence of CAGCGCCCACCAGUCA (SEQ ID NO: 21), optionally wherein R³ comprises an oligonucleotide comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, compositions (e.g., in aqueous solutions) for administration to a subject in the methods described herein further comprise complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, methods provided herein comprise administering to the subject a composition comprising an effective amount of complexes, wherein each complex comprises a structure of formula (I): [R¹]ₙ₁—R², wherein each instance of R¹ in a complex of a composition provided herein comprises a group of the formula (Ib):

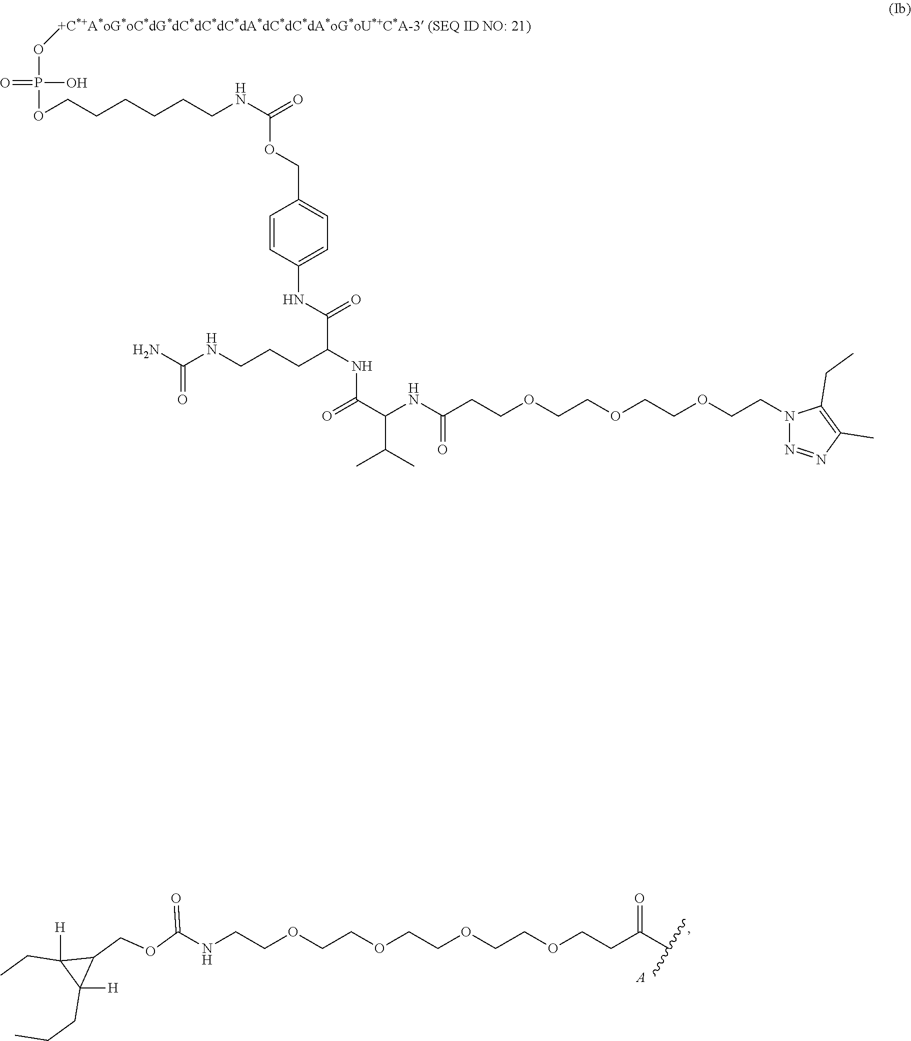

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21);
wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, compositions (e.g., in aqueous solutions) for administration to a subject in the methods described herein further comprise complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, methods provided herein comprise administering to the subject a composition comprising an effective amount of complexes, wherein each complex comprises a structure of formula (I): [R¹]ₙ₁—R², wherein each R¹ of the complexes comprises a group of the formula (Ic):

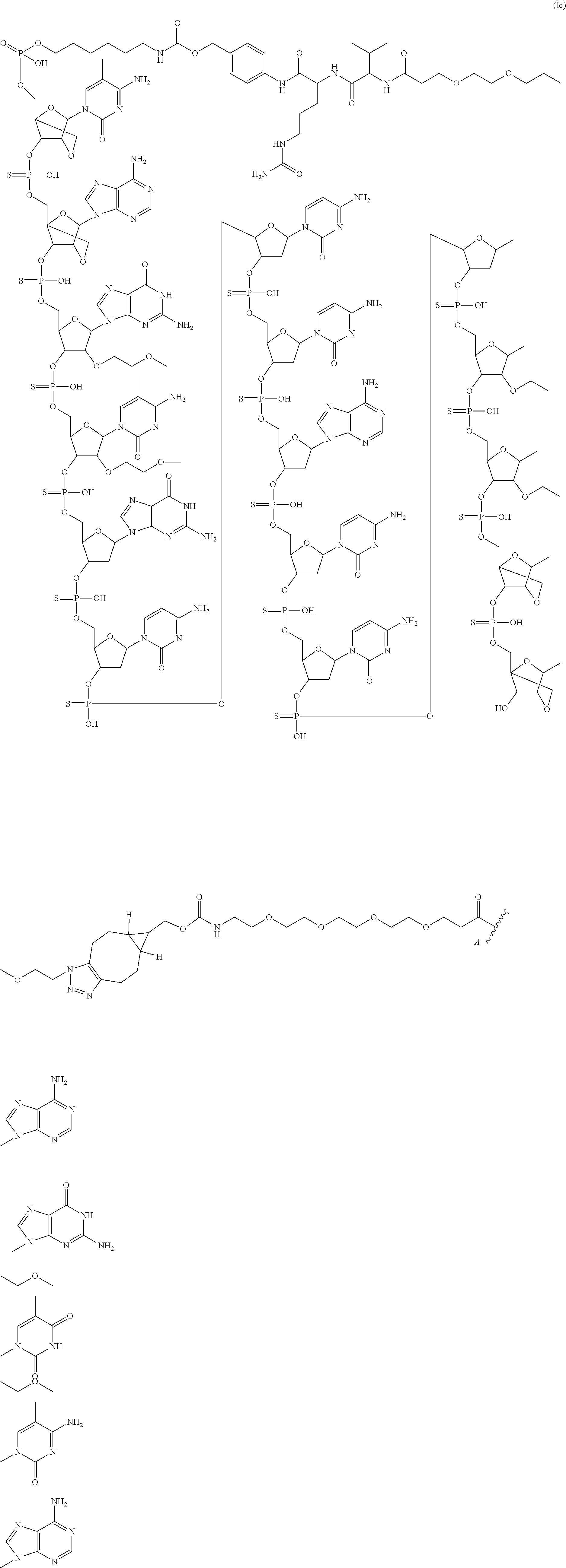

wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, compositions (e.g., in aqueous solutions) for administration to a subject in the methods described herein further comprise complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, methods provided herein comprise administering to the subject a composition comprising an effective amount of muscle targeting complexes, wherein each complex comprises a group of the formula (Id):

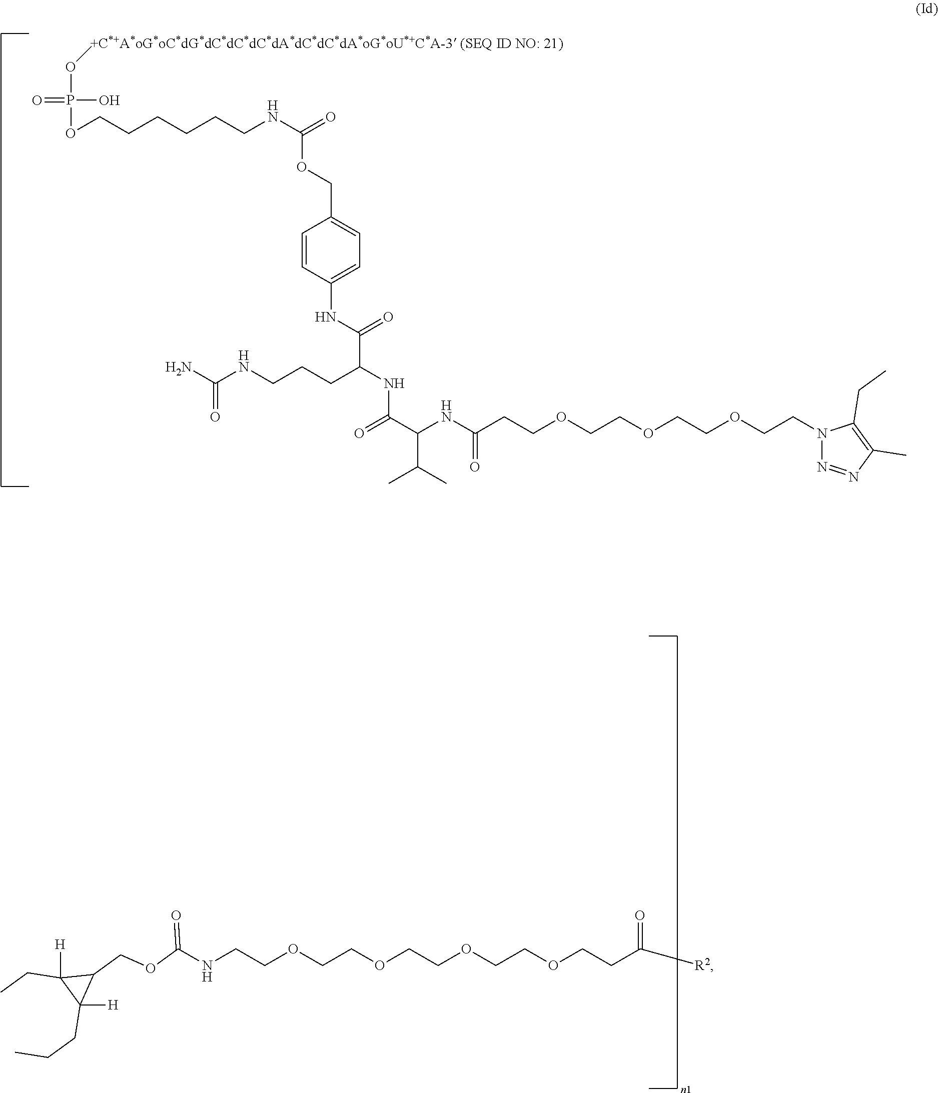

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21);
wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20; and
wherein in each complex n1 is independently an integer of one or greater, optionally wherein the average value of n1 of the complexes of the composition is in the range of 1 to 5, optionally wherein the anti-TfR1 antibody (e.g., Fab) covalently linked via different amino acid residue of the antibody (e.g., Fab), optionally wherein each different amino acid residue is a lysine. In some embodiments, compositions (e.g., in aqueous solutions) for administration to a subject in the methods described herein further comprise complexes in which n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, in any one of the methods described herein, the composition is in an aqueous solution and further comprises tris(hydroxymethyl)aminomethane and sucrose, wherein the tris(hydroxymethyl)aminomethane is present in the aqueous solution at a concentration of 5 mM to 50 mM, the sucrose is present in the aqueous solution at a concentration of 5 w/v % to 15 w/v %. In some embodiments, in any one of the methods described herein, the composition is in an aqueous solution and further comprises tris(hydroxymethyl)aminomethane and sucrose, wherein the tris(hydroxymethyl)aminomethane is present in the aqueous solution at a concentration of or about 25 mM, the sucrose is present in the aqueous solution at a concentration of or about 10 w/v %, and the composition is at a pH of or about 7.5. In some embodiments, the complexes are present in the composition at a concentration in the range of 10 mg/mL to 50 mg/mL (e.g., 10 mg/mL, 15 mg/mL, 20 mg/mL, 25 mg/mL, 30 mg/mL, 35 mg/mL, 40 mg/mL, 45 mg/mL, 50 mg/mL).

In some embodiments, a method described herein comprises administering to a subject a composition comprising an effective amount of complexes comprising a structure of formula (I): [R¹]ₙ₁—R² as described herein (e.g., comprising a group of the formula (Ia), (Ib), (Ic), or (Id)). In some embodiments, in any one of the methods described herein, an effective amount of the complexes provides to the subject an amount of the anti-TfR1 antibody (e.g., Fab) of the complexes per kg of the subject, and the amount of oligonucleotides of the complexes provided to the subject per kg of the subject can be derived using the equation (Equation A) below:

$$oligonucleotide amount ( mg ) = ( \frac{anti - TfR 1 antibody amount ( mg )}{MW of anti - TfR 1 antibody ( \frac{g}{mol} )} ) * average value of n 1 * MW of oligonucleotide ( \frac{g}{mol} ) ,$$

in which “MW” indicates molecular weight (g/mol), and “average value of n1” indicates the average value of n1 of the complexes of the composition.

In some embodiments, a method described herein comprises administering to a subject a composition comprising an effective amount of complexes comprising a structure of formula (I): [R¹]ₙ₁—R² as described herein (e.g., comprising a group of the formula (Ia), (Ib), (Ic), or (Id)). In some embodiments, in any one of the methods described herein, an effective amount of the complexes provides an amount of the oligonucleotides of the complexes to the subject per kg of the subject, and the amount of the anti-TfR1 antibody (e.g., Fab) of the complexes provided to the subject per kg of the subject can be derived using the equation (Equation B) below:

$$anti TfR 1 antibody amount ( mg ) = ( \frac{( \frac{oligonucleotide amount ( mg )}{MW of oligonucleotide ( \frac{g}{mol} )} )}{average value of n 1} ) * MW of anti TfR 1 antibody ( \frac{g}{mol} ) ,$$

in which “MW” indicates molecular weight (g/mol), and “average value of n1” indicates the average value of n1 of the complexes of the composition.

It is to be understood that the average value of n1 need not be an integer and can be a decimal. In some embodiments, the present disclosure contemplates variation in the average value of n1 of up to 30% (e.g., ±up to 30%, ±up to 25%, ±up to 20%, ±up to 15%, ±up to 10%, ±up to 5%, ±up to 3%, or ±up to 1%). In some embodiments, the average value of n1 of the complexes of the composition is in the range of 0.5 to 5 (e.g., 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5 4.5-5, 0.8-1.5, 0.9-1.4, or 1-1.3), with a variation of up to 30% (e.g., ±up to 30%, ±up to 25%, ±up to 20%, ±up to 15%, ±up to 10%, ±up to 5%, ±up to 3%, or ±up to 1%). In some embodiments, a method provided herein comprises administering to the subject a composition comprising an effective amount of complexes comprising a structure of formula (I): [R¹]ₙ₁—R² as described herein (e.g., comprising a group of the formula (Ia), (Ib), (Ic), or (Id)), wherein the average value of n1 of the complexes of the composition is in the range of 0.5 to 5 (e.g., 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5 4.5-5, 0.8-1.5, 0.9-1.4, or 1-1.3), with a variation of up to 30% (e.g., ±up to 30%, ±up to 25%, ±up to 20%, ±up to 15%, ±up to 10%, ±up to 5%, ±up to 3%, or ±up to 1%). In some embodiments, a method provided herein comprises administering to the subject a composition comprising an effective amount of complexes comprising a structure of formula (I): [R¹]ₙ₁—R² as described herein (e.g., comprising a group of the formula (Ia), (Ib), (Ic), or (Id)), wherein the average value of n1 of the complexes of the composition is about 0.5, 0.55, 0.6, 0.65, 0.7, 0.75, 0.8, 0.85, 0.9, 0.95, 1, 1.05, 1.1, 1.15, 1.2, 1.25, 1.3, 1, 35, 1.4, 1.45, or 1.5, with a variation of up to 30% (e.g., ±up to 30%, ±up to 25%, ±up to 20%, ±up to 15%, ±up to 10%, ±up to 5%, ±up to 3%, or ±up to 1%). In some embodiments, a method provided herein comprises administering to the subject a composition comprising an effective amount of complexes comprising a structure of formula (I): [R¹]ₙ₁—R² as described herein (e.g., comprising a group of the formula (Ia), (Ib), (Ic), or (Id)), wherein the average value of n1 of the complexes of the composition is about 1.1 or 1.15, with a variation of up to 30% (e.g., ±up to 30%, ±up to 25%, ±up to 20%, ±up to 15%, ±up to 10%, ±up to 5%, ±up to 3% or ±up to 1%).

It should be understood that any amount of anti-TfR1 antibody (e.g., Fab) provided herein can be represented as an amount of oligonucleotide (e.g., DMPK-targeting oligonucleotide) according to Equation A. As such, it should be understood that description herein relating to providing any amount of anti-TfR1 antibody (e.g., Fab) alternatively can be understood as providing oligonucleotide (e.g., DMPK-targeting oligonucleotide) of a corresponding amount according to Equation A. Conversely, any amount of oligonucleoside (e.g., DMPK-targeting oligonucleotide) provided herein can be represented as an amount of anti-TfR1 antibody (e.g., Fab) according to Equation B. As such, it should be understood that description herein relating to providing any amount of oligonucleoside (e.g., DMPK-targeting oligonucleotide) alternatively can be understood as providing anti-TfR1 antibody (e.g., Fab) of a corresponding amount according to Equation B.

In some embodiments, a method provided herein comprises administering to the subject a composition comprising an effective amount of complexes comprising a structure of formula (I): [R¹]ₙ₁—R² as described herein (e.g., comprising a group of the formula (Ia), (Ib), (Ic), or (Id)), wherein the effective amount of complexes provides to the subject an amount of the anti-TfR1 antibody (e.g., Fab) of the complexes per kg of the subject, and wherein the amount of oligonucleotides provided to the subject per kg of the subject is derived using Equation A provided herein based on the average value of n1 of the complexes of the composition and the amount of anti-TfR1 antibody (e.g., Fab) of the complexes provided to the subject per kg of the subject. In some embodiments, the amount of oligonucleotide varies up to 30% from the amount derived from Equation A (e.g., ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising an effective amount of complexes comprising a structure of formula (I): [R¹]ₙ₁—R² as described herein, wherein the composition comprises an amount of the oligonucleotides of the complexes per kg of the subject, and wherein the amount of anti-TfR1 antibody (e.g., Fab) provided to the subject per kg of the subject is derived using Equation B provided herein based on the average value of n1 of the complexes of the composition and the amount of oligonucleotides of the complexes provided to the subject per kg of the subject. In some embodiments, the amount of anti-TfR1 antibody (e.g., Fab) varies up to 45% from the amount derived from Equation B (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%).

In some embodiments, for purposes of determining the molecular weight of an oligonucleotide, the oligonucleotide is represented by the structure of:

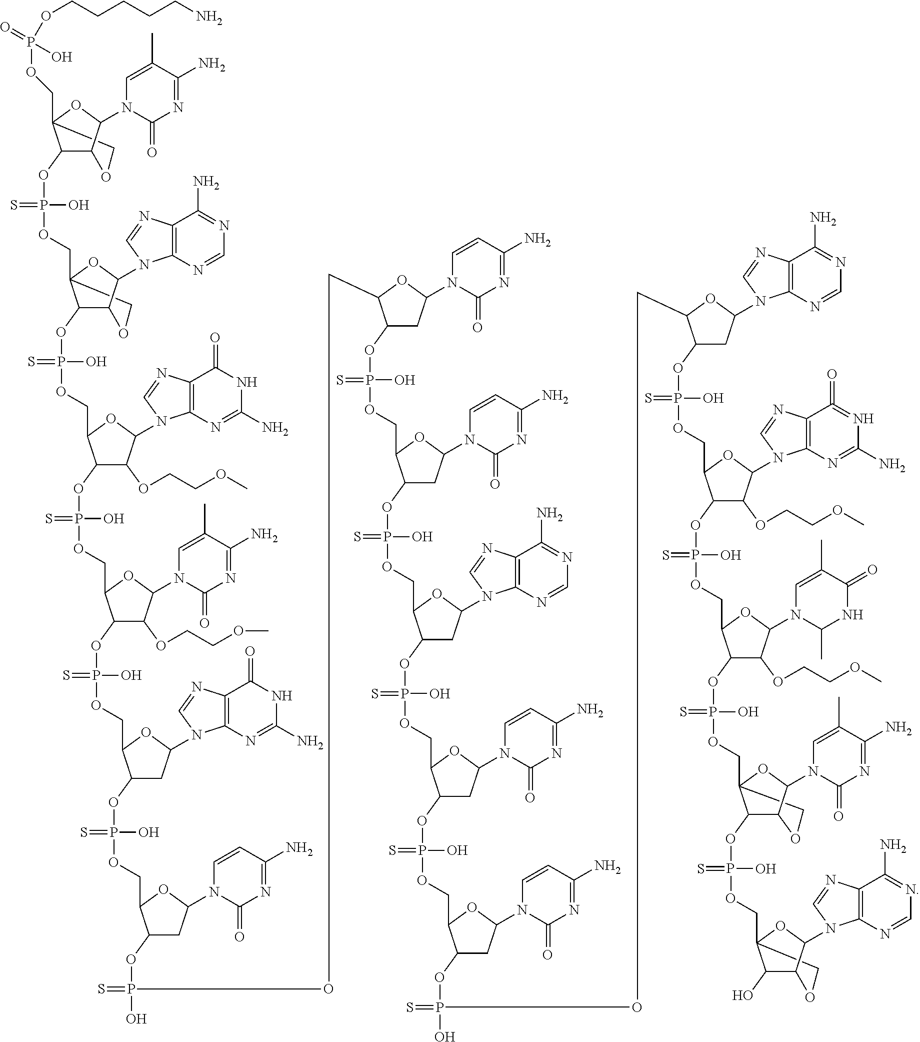

which corresponds to a molecular weight of 5,667 g/mol.

In some embodiments, an anti-TfR1 Fab (e.g., an anti-TfR1 Fab comprising a heavy chain and a light chain provided in Table 2) of a complex used in the methods described herein has a molecular weight of 47,986 g/mol.

Accordingly, in some embodiments, a method described herein comprises administering to a subject a composition comprising an effective amount of complexes comprising a structure of formula (I): [R¹]ₙ₁—R² as described herein (e.g., comprising a group of the formula (Ia), (Ib), (Ic), or (Id)), wherein the effective amount of complexes provides to the subject an amount of the anti-TfR1 Fab of the complexes per kg of the subject, and an amount of oligonucleotide per kg of the subject, wherein the amount of oligonucleotide is:

$$oligonucleotide amount ( mg ) = ( \frac{anti - TfR 1 antibody amount ( mg )}{47986 ( \frac{g}{mol} )} ) * 1.15 * 5667 ( \frac{g}{mol} ) ,$$

provided that the average value of n1 of the complexes of the composition is 1.15. In some embodiments, the amount of oligonucleotide varies up to 30% from the amount derived from Equation A (e.g., ±up to 30%, ±up to 25%, ±up to 20%, ±up to 15%, ±up to 10%, ±up to 5%, ±up to 3%, or ±up to 1%).

In some embodiments, a method described herein comprises administering to a subject a composition comprising an effective amount of complexes comprising a structure of formula (I): [R¹]ₙ₁—R² as described herein (e.g., comprising a group of the formula (Ia), (Ib), (Ic), or (Id)), wherein the effective amount of complexes provides to the subject an amount of the anti-TfR1 Fab of the complexes per kg of the subject, and an amount of oligonucleotide per kg of the subject, wherein the amount of oligonucleotide is:

$$oligonucleotide amount ( mg ) = ( \frac{anti - TfR 1 antibody amount ( mg )}{47968 ( \frac{g}{mol} )} ) * 1.15 * 5667 ( \frac{g}{mol} ) ,$$

provided that the average value of n1 of the complexes of the composition is 1.15. In some embodiments, the amount of oligonucleotide varies up to 30% from the amount derived from Equation A (e.g., ±up to 30%, ±up to 25%, ±up to 20%, ±up to 15%, ±up to 10%, ±up to 5%, ±up to 3%, or ±up to 1%).

In some embodiments, a method described herein comprises administering to a subject a composition comprising an effective amount of complexes comprising a structure of formula (I): [R¹]ₙ₁—R² as described herein, wherein the effective amount of complexes provides to the subject an amount of the oligonucleotides of the complexes per kg of the subject, and an amount of anti-TfR1 Fab per kg of the subject, wherein the amount of anti-TfR1 Fab is:

$$anti TfR 1 Fab amount ( mg ) = ( \frac{( \frac{oligonucleotide amount ( mg )}{5667 ( \frac{g}{mol} )} )}{1.15} ) * 47986 ( \frac{g}{mol} ) ,$$

provided that the average value of n1 of the complexes of the composition is 1.15. In some embodiments, the amount of anti-TfR1 antibody (e.g., Fab) varies up to 45% from the amount derived from Equation B (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%).

In some embodiments, a method described herein comprises administering to a subject a composition comprising an effective amount of complexes comprising a structure of formula (I): [R¹]ₙ₁—R² as described herein, wherein the effective amount of complexes provides to the subject an amount of the oligonucleotides of the complexes per kg of the subject, and an amount of anti-TfR1 Fab per kg of the subject, wherein the amount of anti-TfR1 Fab is:

$$anti TfR 1 Fab amount ( mg ) = ( \frac{( \frac{oligonucleotide amount ( mg )}{5667 ( \frac{g}{mol} )} )}{1.15} ) * 47968 ( \frac{g}{mol} ) ,$$

provided that the average value of n1 of the complexes of the composition is 1.15. In some embodiments, the amount of anti-TfR1 antibody (e.g., Fab) varies up to 45% from the amount derived from Equation B (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the administration occurs one or more times. In some embodiments, the subject is administered a single dose of any one of the compositions comprising an effective amount of the complexes described herein (e.g., complexes comprising a structure of formula (I): [R¹]ₙ₁—R² such as one comprising a group of the formula (Ia), (Ib), (Ic), or (Id)). In some embodiments, the subject is administered multiple doses of any one of the compositions comprising an effective amount of the complexes described herein (e.g., complexes comprising a structure of formula (I): [R¹]ₙ₁—R² such as one comprising a group of the formula (Ia), (Ib), (Ic), or (Id)). In some embodiments, a method described herein comprises administering to the subject a composition comprising an effective amount of the complexes described herein (e.g., complexes comprising a structure of formula (I): [R¹]ₙ₁—R² such as one comprising a group of the formula (Ia), (Ib), (Ic), or (Id)) once a week to once every 16 weeks. For example, in some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, or once every 16 weeks.

In some embodiments, in any one of the methods described herein a composition is administered to a subject during a period of administration. In some embodiments, a period of administration is 1-24 (e.g., 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 1-18, 1-12, 1-8, 1-6, 1-5, 1-4, 1-3, 1-2, 2-18, 2-12, 2-8, 2-6, 2-4, 4-18, 4-12, 4-8, 4-6, 6-18, 6-12, 6-8, 8-18, 8-12, or 12-18) months. In some embodiments, a period of administration is less than 1 month, less than 2 months, less than 3 months, less than 4 months, less than 5 months, less than 6 months, less than 7 months, less than 8 months, less than 9 months, less than 10 months, less than 11 months, less than 12 months, less than 13 months, less than 14 months, less than 15 months, less than 16 months, less than 17 months, or less than 18 months. In some embodiments, a period of administration is 1-20 (e.g., 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 2-18, 2-12, 2-10, 2-5, 4-18, 4-12, 4-8, 6-18, 6-12, or 6-8) years. In some embodiments, a period of administration is less than 1 year, less than 2 years, less than 3 years, less than 4 years, less than 5 years, less than 6 years, less than 7 years, less than 8 years, less than 9 years, less than 10 years, less than 11 years, less than 12 years, less than 13 years, less than 14 years, less than 15 years, less than 16 years, less than 17 years, less than 18 years, less than 19 years, or less than 20 years. In some embodiments, a period of administration is at least 1 year, at least 2 years, at least 3 years, at least 4 years, at least 5 years, at least 6 years, at least 7 years, at least 8 years, at least 9 years, at least 10 years, at least 11 years, at least 12 years, at least 13 years, at least 14 years, at least 15 years, at least 16 years, at least 17 years, at least 18 years, at least 19 years, or at least 20 years. In some embodiments, a period of administration is 1-50 years (e.g., 1-50 years, 2-50 years, 3-50 years, 4-50 years, 5-50 years, 6-50 years, 7-50 years, 8-50 years, 9-50 years, 10⁻⁵⁰ years, 20-50 years, 30-50 years, 40-50 years, 10⁻²⁰ years, 10⁻³⁰ years, 10⁻⁴⁰ years, 20-30 years, 20-40 years, or 30-40 years). In some embodiments, a period of administration is the remainder of the subject's life.

In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, or once every 16 weeks for the remainder of the subject's lifetime. In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 4 weeks for the remainder of the subject's lifetime. In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 8 weeks for the remainder of the subject's lifetime. In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 12 weeks for the remainder of the subject's lifetime.

In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once a week to once every four weeks (e.g., once a week, once every 2 weeks, once every 3 weeks, or once every 4 weeks) during a first period of administration, followed by administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 4 weeks to once every 16 weeks (e.g., once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, or once every 16 weeks) during a second period of administration. In some embodiments, the first period of administration is 2-24 weeks (e.g., 2-24, 2-20, 2-26, 2-12, 2-8, 2-4, 4-24, 4-20, 4-16, 4-12, 4-8, 8-24, 8-20, 8-16, 8-12, 12-24, 12-20, 12-16, 16-24, 16-20, or 20-24 weeks). In some embodiments, the first period of administration is 2, 3, 4, 5, 6, 7, 8, 9, 10, 11, 12, 13, 14, 15, 16, 17, 18, 19, 20, 21, 22, 23, or 24 weeks. In some embodiments, the second period of administration is 4 weeks to the remainder of the subject's lifetime (e.g., 4, 8, 12, 16, 18, 20, 22, 24, 28, 32, 36, 40, 44, 48, 52, 56, 60, 64, 68, 72, 76, 80, 84, 88, 92, 96 weeks or longer to the remainder of the subject's lifetime). In some embodiments, the first period of administration is 8 weeks, and the second period of administration is 16 weeks. In some embodiments, the first period of administration is 8 weeks, and the second period of administration is the remainder of the subject's lifetime.

In some embodiments, each administration during the first period of administration provides the subject with a same amount of complexes (e.g., complexes comprising a structure of formula (I): [R¹]ₙ₁—R² such as one comprising a group of the formula (Ia), (Ib), (Ic), or (Id)) as each administration during the second period of administration. In some embodiments, each administration during the first period of administration provides the subject with a different amount of complexes (e.g., complexes comprising a structure of formula (I): [R¹]ₙ₁—R² such as one comprising a group of the formula (Ia), (Ib), (Ic), or (Id)) from each administration during the second period of administration.

In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 4 weeks during a first period of administration of 8 weeks, 12 weeks, or 16 weeks, followed by administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 8 weeks during a second period of administration of 16 weeks, 24 weeks, 32 weeks, 40 weeks, 48 weeks, 56 weeks, 64 weeks, 72 weeks, 80 weeks, 88 weeks, 96 weeks, 104 weeks, 112 weeks, 120 weeks, 128 weeks, 136 weeks, 144 weeks, 152 weeks, 160 weeks, or longer, or the remainder of the subject's lifetime, wherein each administration during the first period of administration provides the subject with a same amount of complexes (e.g., complexes comprising a structure of formula (I): [R¹]ₙ₁—R² such as one comprising a group of the formula (Ia), (Ib), (Ic), or (Id)) as each administration during the second period of administration.

In some embodiments, a method described herein comprises administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 4 weeks during a first period of administration of 8 weeks, 12 weeks, or 16 weeks, followed by administering to the subject a composition comprising an effective amount of complexes described herein (e.g., complexes comprising a structure of formula (I): [R¹]ₙ₁—R² such as one comprising a group of the formula (Ia), (Ib), (Ic), or (Id)) once every 8 weeks during a second period of administration of 16 weeks, 24 weeks, 32 weeks, 40 weeks, 48 weeks, 56 weeks, 64 weeks, 72 weeks, 80 weeks, 88 weeks, 96 weeks, 104 weeks, 112 weeks, 120 weeks, 128 weeks, 136 weeks, 144 weeks, 152 weeks, 160 weeks, or longer, or the remainder of the subject's lifetime, wherein each administration during the first period of administration provides the subject with a different amount of complexes (e.g., complexes comprising a structure of formula (I): [R¹]ₙ₁—R² such as one comprising a group of the formula (Ia), (Ib), (Ic), or (Id)) from each administration during the second period of administration.

In some embodiments, in any one of the methods described herein, additional administrations may be given to the subject during any period of administration.

In some embodiments, in any one of the methods described herein, the effective amount of complexes (e.g., complexes comprising a structure of formula (I): [R¹]ₙ₁—R² such as one comprising a group of the formula (Ia), (Ib), (Ic), or (Id)) provides to the subject 2 mg to 220 mg (e.g., 2 mg to 220 mg, 5 mg to 220 mg, 5 mg to 210 mg, 5 mg to 200 mg, 5 mg to 190 mg, 5 mg to 180 mg, 5 mg to 170 mg, 5 mg to 160 mg, 5 mg to 150 mg, 5 mg to 140 mg, 5 mg to 130 mg, 5 mg to 120 mg, 5 mg to 110 mg, 5 mg to 100 mg, 5 mg to 90 mg, 5 mg to 80 mg, 5 mg to 75 mg, 5 mg to 70 mg, 5 mg to 60 mg, 5 mg to 50 mg, 5 mg to 40 mg, 5 mg to 30 mg, 5 mg to 20 mg, 5 mg to 10 mg, 8 mg to 220 mg, 8 mg to 210 mg, 8 mg to 200 mg, 8 mg to 190 mg, 8 mg to 180 mg, 8 mg to 170 mg, 8 mg to 160 mg, 8 mg to 150 mg, 8 mg to 140 mg, 8 mg to 130 mg, 8 mg to 120 mg, 8 mg to 110 mg, 8 mg to 100 mg, 8 mg to 90 mg, 8 mg to 80 mg, 8 mg to 75 mg, 8 mg to 70 mg, 8 mg to 60 mg, 8 mg to 50 mg, 8 mg to 40 mg, 8 mg to 35 mg, 8 mg to 30 mg, 8 mg to 25 mg, 8 mg to 20 mg, 8 mg to 15 mg, 8 mg to 13 mg, 10 mg to 150 mg, 10 mg to 140 mg, 10 mg to 130 mg, 10 mg to 120 mg, 10 mg to 110 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 13 mg to 150 mg, 13 mg to 140 mg, 13 mg to 130 mg, 13 mg to 120 mg, 13 mg to 110 mg, 13 mg to 100 mg, 13 mg to 90 mg, 13 mg to 80 mg, 13 mg to 75 mg, 13 mg to 70 mg, 13 mg to 60 mg, 13 mg to 50 mg, 13 mg to 40 mg, 13 mg to 35 mg, 13 mg to 30 mg, 13 mg to 25 mg, 13 mg to 20 mg, 13 mg to 15 mg, 15 mg to 150 mg, 15 mg to 140 mg, 15 mg to 130 mg, 15 mg to 120 mg, 15 mg to 110 mg, 15 mg to 100 mg, 15 mg to 90 mg, 15 mg to 80 mg, 15 mg to 75 mg, 15 mg to 70 mg, 15 mg to 60 mg, 15 mg to 50 mg, 15 mg to 40 mg, 15 mg to 35 mg, 15 mg to 30 mg, 15 mg to 25 mg, 15 mg to 20 mg, 20 mg to 100 mg, 20 mg to 90 mg, 20 mg to 80 mg, 20 mg to 75 mg, 20 mg to 70 mg, 20 mg to 60 mg, 20 mg to 50 mg, 20 mg to 40 mg, 20 mg to 30 mg, 20 mg to 25 mg, 25 mg to 90 mg, 25 mg to 80 mg, 25 mg to 75 mg, 25 mg to 70 mg, 25 mg to 60 mg, 25 mg to 50 mg, 25 mg to 40 mg, 25 mg to 35 mg, 25 mg to 30 mg, 30 mg to 100 mg, 30 mg to 90 mg, 30 mg to 80 mg, 30 mg to 75 mg, 30 mg to 70 mg, 30 mg to 60 mg, 30 mg to 50 mg, 30 mg to 40 mg, 40 mg to 100 mg, 40 mg to 90 mg, 40 mg to 80 mg, 40 mg to 75 mg, 40 mg to 70 mg, 40 mg to 60 mg, 40 mg to 50 mg, 50 mg to 100 mg, 50 mg to 90 mg, 50 mg to 80 mg, 50 mg to 75 mg, 50 mg to 70 mg, 50 mg to 60 mg, 60 mg to 100 mg, 60 mg to 90 mg, 60 mg to 80 mg, 60 mg to 75 mg, or 60 mg to 70 mg) of the anti-TfR1 antibody (e.g., Fab) of the complexes per kg of the subject. In some embodiments, in any one of the methods described herein, the effective amount of complexes (e.g., complexes comprising a structure of formula (I): [R¹]ₙ₁—R² such as one comprising a group of the formula (Ia), (Ib), (Ic), or (Id)) provides to the subject about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, or 220 mg of the anti-TfR1 antibody (e.g., Fab) of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%).

In some embodiments, in any one of the methods described herein, the effective amount of complexes (e.g., complexes comprising a structure of formula (I): [R¹]ₙ₁—R² such as one comprising a group of the formula (Ia), (Ib), (Ic), or (Id)) provides to the subject 0.5 mg to 20 mg (e.g., 0.5 mg to 20 mg, 1 mg to 20 mg, 1 mg to 15 mg, 1 mg to 12 mg, 1 mg to 10 mg, 1 mg to 9 mg, 1 mg to 8 mg, 1 mg to 7 mg, 1 mg to 6 mg, 1 mg to 5 mg, 1 mg to 4 mg, 1 mg to 3 mg, 1 mg to 2 mg, 2 mg to 20 mg, 2 mg to 15 mg, 2 mg to 12 mg, 2 mg to 10 mg, 2 mg to 9 mg, 2 mg to 8 mg, 2 mg to 7 mg, 2 mg to 6 mg, 2 mg to 5 mg, 2 mg to 4 mg, 2 mg to 3 mg, 3 mg to 20 mg, 3 mg to 15 mg, 3 mg to 12 mg, 3 mg to 10 mg, 3 mg to 9 mg, 3 mg to 8 mg, 3 mg to 7 mg, 3 mg to 6 mg, 3 mg to 5 mg, 3 mg to 4 mg, 3.5 mg to 20 mg, 3.5 mg to 15 mg, 3.5 mg to 12 mg, 3.5 mg to 10 mg, 3.5 mg to 9 mg, 3.5 mg to 8 mg, 3.5 mg to 7 mg, 3.5 mg to 6 mg, 3.5 mg to 5 mg, 3.5 mg to 4 mg, 4 mg to 20 mg, 4 mg to 15 mg, 4 mg to 12 mg, 4 mg to 10 mg, 4 mg to 9 mg, 4 mg to 8 mg, 4 mg to 7 mg, 4 mg to 6 mg, 4 mg to 5 mg, 5 mg to 20 mg, 5 mg to 15 mg, 5 mg to 12 mg, 5 mg to 10 mg, 5 mg to 9 mg, 5 mg to 8 mg, 5 mg to 7 mg, 5 mg to 6 mg, 6 mg to 20 mg, 6 mg to 15 mg, 6 mg to 12 mg, 6 mg to 10 mg, 6 mg to 9 mg, 6 mg to 8 mg, 6 mg to 7 mg, 7 mg to 20 mg, 7 mg to 15 mg, 7 mg to 12 mg, 7 mg to 10 mg, 7 mg to 9 mg, 7 mg to 8 mg, 8 mg to 20 mg, 8 mg to 15 mg, 8 mg to 12 mg, 8 mg to 10 mg, 8 mg to 9 mg, 9 mg to 20 mg, 9 mg to 15 mg, 9 mg to 12 mg, 9 mg to 10 mg, 10 mg to 20 mg, 10 mg to 15 mg, 10 mg to 12 mg, 12 mg to 20 mg, 12 mg to 15 mg, or 15 mg to 20 mg) of the oligonucleotides of the complexes per kg of the subject. In some embodiments, in any one of the methods described herein, the effective amount of complexes (e.g., complexes comprising a structure of formula (I): [R¹]ₙ₁—R² such as one comprising a group of the formula (Ia), (Ib), (Ic), or (Id)) provides to the subject about 0.5 mg, 1 mg, 1.1 mg, 1.5 mg, 1.8 mg, 2 mg, 2.5 mg, 3 mg, 3.4 mg, 3.5 mg, 4 mg, 4.1 mg, 4.5 mg, 5 mg, 5.5 mg, 6 mg, 6.5 mg, 6.8 mg, 7 mg, 7.5 mg, 8 mg, 8.2 mg, 8.5 mg, 9 mg, 9.5 mg, 10 mg, 10.2 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, or 20 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 10 mg to 110 mg (e.g., 10 mg to 110 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 110 mg, 20 mg to 100 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 110 mg, 30 mg to 100 mg, 30 mg to 90 mg, 30 mg to 80 mg, 30 mg to 70 mg, 30 mg to 60 mg, 30 mg to 50 mg, 30 mg to 40 mg, 40 mg to 110 mg, 40 mg to 100 mg, 40 mg to 90 mg, 40 mg to 80 mg, 40 mg to 70 mg, 40 mg to 60 mg, 40 mg to 50 mg, 50 mg to 110 mg, 50 mg to 100 mg, 50 mg to 90 mg, 50 mg to 80 mg, 50 mg to 70 mg, 50 mg to 60 mg, 60 mg to 110 mg, 60 mg to 100 mg, 60 mg to 90 mg, 60 mg to 80 mg, 60 mg to 70 mg, 70 mg to 110 mg, 70 mg to 100 mg, 70 mg to 90 mg, 70 mg to 80 mg, 80 mg to 110 mg, 80 mg to 100 mg, 80 mg to 90 mg, 90 mg to 110 mg, 90 mg to 100 mg, 100 mg to 110 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 13 mg, about 25 mg, about 50 mg, or about 75 mg of the anti-TfR1 antibodies of the complexes per kg of the subject. In some embodiments, the effective amount (e.g., of each administration) provides to the subject about 29 mg or about 37 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%. ±up to 25%, ±up to 20%, ±up to 15%. ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 10 mg to 20 mg (e.g., 10 mg to 20 mg, 10.5 mg to 18 mg, 11 mg to 16 mg, 11.5 mg to 15 mg, 12 mg to 14 mg, or 12.5 mg to 13 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 13 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 18 mg to 36 mg (e.g., 18 mg to 36 mg, 19 mg to 33 mg, 20 mg to 30 mg, 22 mg to 27 mg, or 23 mg to 26 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 25 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 20 mg to 43 mg (e.g., 20 mg to 43 mg, 22 mg to 40 mg, 24 mg to 34 mg, 26 mg to 32 mg, or 28 mg to 30 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 29 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%. ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 26 mg to 53 mg (e.g., 26 mg to 53 mg, 29 mg to 50 mg, 31 mg to 45 mg, 33 mg to 41 mg, or 35 mg to 40 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 37 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 36 mg to 72 mg (e.g., 36 mg to 72 mg, 37 mg to 69 mg, 38 mg to 66 mg, 39 mg to 63 mg, 40 mg to 60 mg, 41 mg to 57 mg, 42 mg to 54 mg, or 45 mg to 51 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 50 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 55 mg to 110 mg (e.g., 55 mg to 110 mg, 58 mg to 100 mg, 61 mg to 95 mg, 64 mg to 90 mg, 67 mg to 85 mg, or 70 mg to 80 mg, or 73 mg to 76 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 75 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 5 mg to 90 mg (e.g., 5 mg to 90 mg, 5 mg to 80 mg, 5 mg to 70 mg, 5 mg to 60 mg, 5 mg to 50 mg, 5 mg to 40 mg, 5 mg to 30 mg, 5 mg to 20 mg, 5 mg to 10 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 90 mg, 30 mg to 80 mg, 30 mg to 70 mg, 30 mg to 60 mg, 30 mg to 50 mg, 30 mg to 40 mg, 40 mg to 90 mg, 40 mg to 80 mg, 40 mg to 70 mg, 40 mg to 60 mg, 40 mg to 50 mg, 50 mg to 90 mg, 50 mg to 80 mg, 50 mg to 70 mg, 50 mg to 60 mg, 60 mg to 90 mg, 60 mg to 80 mg, 60 mg to 70 mg, 70 mg to 90 mg, 70 mg to 80 mg, or 80 mg to 90 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 8 mg, about 15 mg, about 30 mg, or about 60 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%±up to 40%, ±up to 35%, ±up to 30%±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 6 mg to 12 mg (e.g., 6 mg to 12 mg, 6.5 mg to 11 mg, 7 mg to 10 mg, 7.5 mg to 9 mg, or 8 mg to 8.5 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 8 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 11 mg to 22 mg (e.g., 11 mg to 22 mg, 11.5 mg to 20 mg, 12 mg to 19 mg, 12.5 mg to 18 mg, 13 mg to 17 mg, 14 mg to 16 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 15 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 22 mg to 44 mg (e.g., 22 mg to 44 mg, 23 mg to 42 mg, 24 mg to 40 mg, 25 mg to 38 mg, 26 mg to 36 mg, 27 mg to 34 mg, 28 mg to 32 mg, or 29 mg to 31 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 30 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 44 mg to 88 mg (e.g., 44 mg to 88 mg, 46 mg to 84 mg, 48 mg to 80 mg, 50 mg to 76 mg, 52 mg to 72 mg, 54 mg to 68 mg, 56 mg to 64 mg, or 58 mg to 62 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 60 mg of the anti-TfR1 antibodies of the complexes per kg of the subject. In some embodiments, with respect to any of the preceding amounts of anti-TfR1 antibody (e.g., Fab), the values can vary by up to 45% (e.g., ±up to 45%, ±up to 40%, ±up to 35%, ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 36.8 mg to 58.9 mg (e.g., 36.8 mg to 58.9 mg, 36.8 mg to 51.5 mg, 36.8 mg to 44.2 mg, 44.2 mg to 58.9 mg, 44.2 mg to 51.5 mg, or 51.5 mg to 58.9 mg) of the anti-TfR1 antibodies of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 36.8 mg of the anti-TfR1 antibodies of the complexes per kg of the subject. In some embodiments, with respect to any of the preceding amounts of anti-TfR1 antibodies, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 1 mg to 14 mg (e.g., 1 mg to 14 mg, 1 mg to 13 mg, 1 mg to 12 mg, 1 mg to 11 mg, 1 mg to 10 mg, 1 mg to 9 mg, 1 mg to 8 mg, 1 mg to 7 mg, 1 mg to 6 mg, 1 mg to 5 mg, 1 mg to 4 mg, 1 mg to 3 mg, 1 mg to 2 mg, 2 mg to 14 mg, 2 mg to 13 mg, 2 mg to 12 mg, 2 mg to 11 mg, 2 mg to 10 mg, 2 mg to 9 mg, 2 mg to 8 mg, 2 mg to 7 mg, 2 mg to 6 mg, 2 mg to 5 mg, 2 mg to 4 mg, 2 mg to 3 mg, 3 mg to 14 mg, 3 mg to 13 mg, 3 mg to 12 mg, 3 mg to 11 mg, 3 mg to 10 mg, 3 mg to 9 mg, 3 mg to 8 mg, 3 mg to 7 mg, 3 mg to 6 mg, 3 mg to 5 mg, 3 mg to 4 mg, 4 mg to 14 mg, 4 mg to 13 mg, 4 mg to 12 mg, 4 mg to 11 mg, 4 mg to 10 mg, 4 mg to 9 mg, 4 mg to 8 mg, 4 mg to 7 mg, 4 mg to 6 mg, 4 mg to 5 mg, 5 mg to 14 mg, 5 mg to 13 mg, 5 mg to 12 mg, 5 mg to 11 mg, 5 mg to 10 mg, 5 mg to 9 mg, 5 mg to 8 mg, 5 mg to 7 mg, 5 mg to 6 mg, 6 mg to 14 mg, 6 mg to 13 mg, 6 mg to 12 mg, 6 mg to 11 mg, 6 mg to 10 mg, 6 mg to 9 mg, 6 mg to 8 mg, 6 mg to 7 mg, 7 mg to 14 mg, 7 mg to 13 mg, 7 mg to 12 mg, 7 mg to 11 mg, 7 mg to 10 mg, 7 mg to 9 mg, 7 mg to 8 mg, 8 mg to 14 mg, 8 mg to 13 mg, 8 mg to 12 mg, 8 mg to 11 mg, 8 mg to 10 mg, 8 mg to 9 mg, 9 mg to 14 mg, 9 mg to 13 mg, 9 mg to 12 mg, 9 mg to 11 mg, 9 mg to 10 mg, 10 mg to 14 mg, 10 mg to 13 mg, 10 mg to 12 mg, 10 mg to 11 mg, 11 mg to 14 mg, 11 mg to 13 mg, 11 mg to 12 mg, 12 mg to 14 mg, 12 mg to 13 mg, or 13 mg to 14 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 1.8 mg, about 3.4 mg, about 6.8 mg, or about 10.2 mg of the oligonucleotides of the complexes per kg of the subject. In some embodiments, the effective amount (e.g., of each administration) provides to the subject about 4 mg or about 5 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]l-R² such as one comprising a group of the formula (Ia), (Ib), (Ic), or (Id)), wherein the effective amount (e.g., of each administration) provides to the subject 1.2 mg to 2.4 mg (e.g., 1.2 mg to 2.4 mg, 1.1 mg to 2.3 mg, 1.2 mg to 2.2 mg, 1.3 mg to 2.1 mg, 1.4 mg to 2 mg, 1.5 mg to 1.9 mg, or 1.6 mg to 1.85 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 1.8 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 2.2 mg to 4.5 mg (e.g., 2.2 mg to 4.5 mg, 2.4 mg to 4.4 mg, 2.5 mg to 4.3 mg, 2.6 mg to 4.2 mg, 2.7 mg to 4.1 mg, 2.8 mg to 4 mg, 2.9 mg to 3.9 mg, 3 mg to 3.8 mg, 3.1 mg to 3.7 mg, 3.2 mg to 3.6 mg, or 3.3 mg to 3.5 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 3.4 mg of the oligonucleotides of the complexes per kg of the subject. In some embodiments, with respect to any of the preceding amounts of oligonucleotides, the values can vary by up to 30% (e.g., ±up to 30%. ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 2.7 mg to 5.3 mg (e.g., 2.7 mg to 5.3 mg, 2.8 mg to 5.8 mg, 3 mg to 5.5 mg, 3.2 mg to 5 mg, or 3.8 mg to 4.2 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 4 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 3 mg to 7 mg (e.g., 3 mg to 7 mg, 3.5 mg to 6.6 mg, 4 mg to 6 mg, 4.2 mg to 5.8 mg, or 4.8 mg to 5.2 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 5 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 4.5 mg to 9 mg (e.g., 4.5 mg to 9 mg, 4.7 mg to 8.8 mg, 4.9 mg to 8.6 mg, 5.1 mg to 8.4 mg, 5.3 mg to 8.2 mg, 5.5 mg to 8 mg, 5.7 mg to 7.8 mg, 5.9 mg to 7.6 mg, 6.1 mg to 7.4 mg, 6.3 mg to 7.2 mg, 6.5 mg to 7 mg, or 6.6 mg to 6.8 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 6.8 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 7 mg to 14 mg (e.g., 7 mg to 14 mg, 7.5 mg to 13.5 mg, 8 mg to 13 mg, 8.5 mg to 12.5 mg, 9 mg to 12 mg, 9.5 mg to 11.5 mg, or 10 mg to 11 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 10.2 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 0.5 mg to 12 mg (e.g., 0.5 mg to 12 mg, 0.5 mg to 12 mg, 0.5 mg to 11 mg, 0.5 mg to 10 mg, 0.5 mg to 9 mg, 0.5 mg to 8 mg, 0.5 mg to 7 mg, 0.5 mg to 6 mg, 0.5 mg to 5 mg, 0.5 mg to 4 mg, 0.5 mg to 3 mg, 0.5 mg to 2 mg, 0.5 mg to 1 mg, 1 mg to 12 mg, 1 mg to 11 mg, 1 mg to 10 mg, 1 mg to 9 mg, 1 mg to 8 mg, 1 mg to 7 mg, 1 mg to 6 mg, 1 mg to 5 mg, 1 mg to 4 mg, 1 mg to 3 mg, 1 mg to 2 mg, 2 mg to 12 mg, 2 mg to 11 mg, 2 mg to 10 mg, 2 mg to 9 mg, 2 mg to 8 mg, 2 mg to 7 mg, 2 mg to 6 mg, 2 mg to 5 mg, 2 mg to 4 mg, 2 mg to 3 mg, 3 mg to 12 mg, 3 mg to 11 mg, 3 mg to 10 mg, 3 mg to 9 mg, 3 mg to 8 mg, 3 mg to 7 mg, 3 mg to 6 mg, 3 mg to 5 mg, 3 mg to 4 mg, 4 mg to 12 mg, 4 mg to 11 mg, 4 mg to 10 mg, 4 mg to 9 mg, 4 mg to 8 mg, 4 mg to 7 mg, 4 mg to 6 mg, 4 mg to 5 mg, 5 mg to 12 mg, 5 mg to 11 mg, 5 mg to 10 mg, 5 mg to 9 mg, 5 mg to 8 mg, 5 mg to 7 mg, 5 mg to 6 mg, 6 mg to 12 mg, 6 mg to 11 mg, 6 mg to 10 mg, 6 mg to 9 mg, 6 mg to 8 mg, 6 mg to 7 mg, 7 mg to 12 mg, 7 mg to 11 mg, 7 mg to 10 mg, 7 mg to 9 mg, 7 mg to 8 mg, 8 mg to 12 mg, 8 mg to 11 mg, 8 mg to 10 mg, 8 mg to 9 mg, 9 mg to 12 mg, 9 mg to 11 mg, 9 mg to 10 mg, 10 mg to 12 mg, 10 mg to 11 mg, or 11 mg to 12 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 1.1 mg, about 2 mg, about 4.1 mg, or about 8.2 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 0.7 mg to 1.5 mg (e.g., 0.7 mg to 1.5 mg, 0.8 mg to 1.4 mg, 0.9 mg to 1.3 mg, or 1 mg to 1.2 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 1.1 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 1.4 mg to 2.7 mg (e.g., 1.4 mg to 2.7 mg, 1.5 mg to 2.6 mg, 1.6 mg to 2.5 mg, 1.7 mg to 2.4 mg, 1.8 mg to 2.3 mg, 1.9 mg to 2.2 mg, or 1.95 mg to 2.1 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 2 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 2.7 mg 5.4 mg (e.g., 2.7 mg to 5.4 mg, 2.9 mg to 5.2 mg, 3.1 mg to 5 mg, 3.3 mg to 4.8 mg, 3.5 mg to 4.6 mg, 3.7 mg to 4.3 mg, 3.9 mg to 4.2 mg, or 4 mg to 4.15 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 4.1 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering to a subject a composition an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 5 mg to 11 mg (e.g., 5 mg to 11 mg, 5.5 mg to 10.5 mg, 6 mg to 10 mg, 6.5 mg to 9.5 mg, 7 mg to 9 mg, 7.5 mg to 8.5 mg, or 8 mg to 8.3 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 8.2 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3% or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

It should be understood that once every 4 weeks is substantially similar to once a month, once every 8 weeks is substantially similar to once every two months, once every 12 weeks is substantially similar to once every three months, and once every 16 weeks is substantially similar to once every four months. As such, in some embodiments, once every 4 weeks can mean once a month; once every 8 weeks can mean once every two months; once every 12 weeks can mean once every three months; and once every 16 weeks can mean once every four months. Similarly, in some embodiments, once every 4 weeks can mean 12 times per year; once every 8 weeks can mean 6 times per year; and once every 12 weeks can mean 4 times per year.

In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject 5 mg to 8 mg (e.g., 5 mg to 8 mg, 5 mg to 7 mg, 5 mg to 6 mg, 6 mg to 8 mg, 6 mg to 7 mg, or 7 mg to 8 mg) of the oligonucleotides of the complexes per kg of the subject. In some embodiments, a method described herein comprises administering to a subject a composition comprising an effective amount of complexes described herein (e.g., complexes comprising a structure of formula (I): [R¹]ₙ₁—R² such as one comprising a group of the formula (Ia), (Ib), (Ic), or (Id)), wherein the effective amount (e.g., of each administration) provides to the subject about 5 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, the composition is administered once every 4 weeks (e.g., for a period of time such as the remainder of the subject's lifetime). In some embodiments, the composition is administered once every 8 weeks (e.g., for a period of time such as the remainder of the subject's lifetime). In some embodiments, the composition is administered once every 12 weeks (e.g., for a period of time such as the remainder of the subject's lifetime). In some embodiments, the composition is administered once every 4 weeks during a first period of administration (e.g., 8 weeks, 12 weeks, or 16 weeks), and subsequently administered once every 8 weeks during a second period of administration (e.g., 16 weeks to the remainder of the subject's lifetime).

In some embodiments, a method described herein comprises administering intravenously (e.g., via intravenous infusion) an effective amount of a composition comprising complexes (e.g., in an aqueous solution), wherein each complex comprises a structure of formula (I): [R¹]ₙ₁—R², wherein each R¹ comprises a group of the formula (Ia):

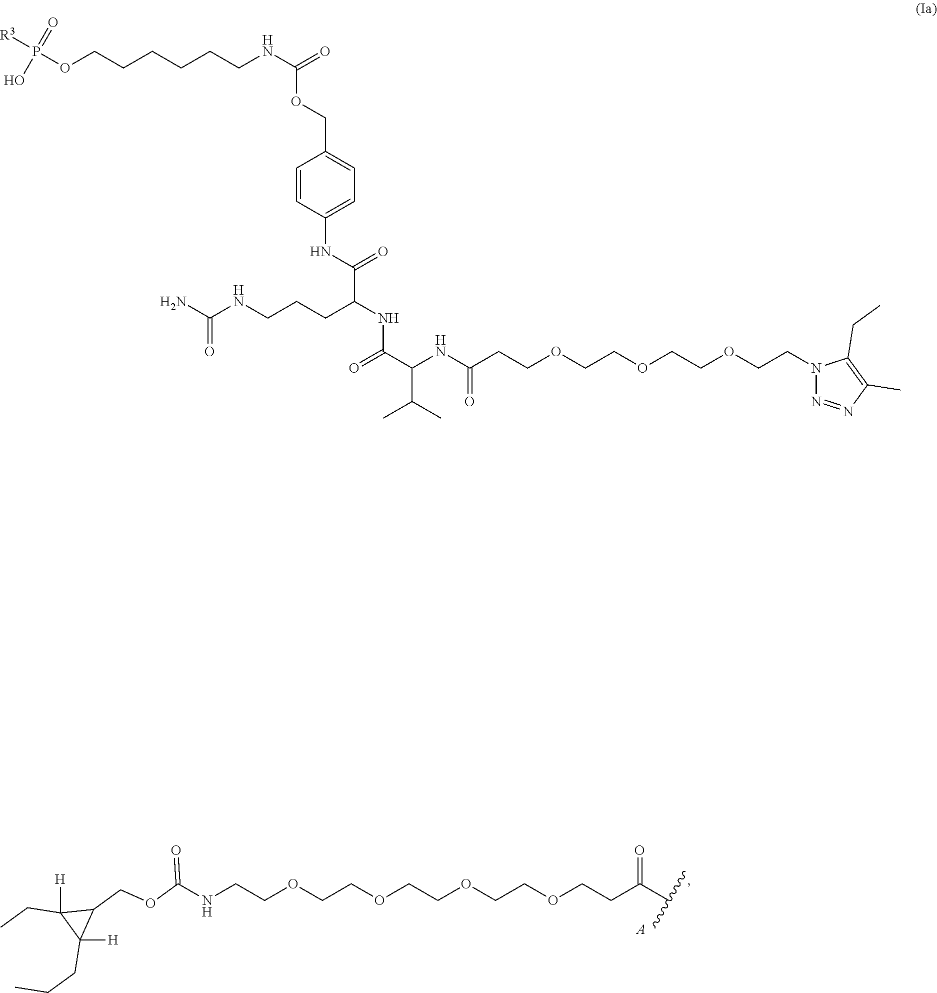

in which R³ comprises an oligonucleotide comprising a nucleobase sequence of CAGCGCCCACCAGUCA (SEQ ID NO: 21), optionally wherein R³ comprises an oligonucleotide comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5; and
wherein the effective amount of each administration provides to the subject 5 mg to 110 mg (e.g., 8 mg to 60 mg or 13 mg to 75 mg) of the anti-TfR1 antibody (e.g., Fab) of the complexes per kg of the subject. In some embodiments, the effective amount of each administration provides to the subject about 13 mg, about 25 mg, about 50 mg, or about 75 mg of the anti-TfR1 antibody (e.g., Fab) of the complexes per kg of the subject. In some embodiments, the effective amount of each administration provides to the subject about 5 mg, about 15 mg, about 30 mg, or about 60 mg of the anti-TfR1 antibody (e.g., Fab) of the complexes per kg of the subject. In some embodiments, the subject is administered the composition once every four weeks or once every eight weeks. In some embodiments, the subject is administered the composition once every 4 weeks during a first period of administration (e.g., 8 weeks, 12 weeks, or 16 weeks), and subsequently administered the composition once every 8 weeks during a second period of administration (e.g., 16 weeks to the remainder of the subject's lifetime). In some embodiments, a composition comprising complexes for administration to a subject in the methods described herein further comprises complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, a method described herein comprises administering intravenously (e.g., via intravenous infusion) an effective amount of a composition comprising complexes (e.g., in an aqueous solution), wherein each complex comprises a structure of formula (I): [R¹]ₙ₁—R², wherein each R¹ comprises a group of the formula (Ib):

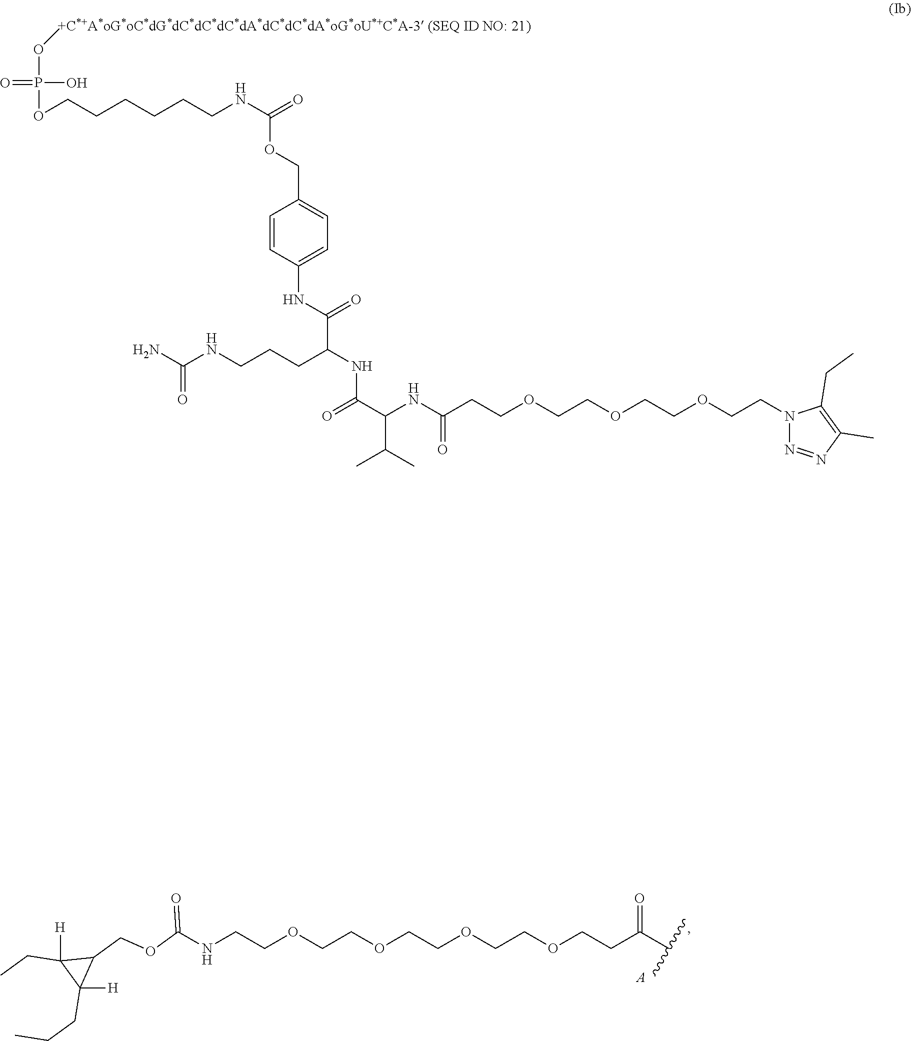

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21);
wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5;
wherein the effective amount of each administration provides to the subject 5 mg to 110 mg (e.g., 8 mg to 60 mg or 13 mg to 75 mg) of the anti-TfR1 antibody (e.g., Fab) of the complexes per kg of the subject. In some embodiments, the effective amount of each administration provides to the subject about 13 mg, about 25 mg, about 50 mg, or about 75 mg of the anti-TfR1 antibody (e.g., Fab) of the complexes per kg of the subject. In some embodiments, the effective amount of each administration provides to the subject about 5 mg, about 15 mg, about 30 mg, or about 60 mg of the anti-TfR1 antibody (e.g., Fab) of the complexes per kg of the subject. In some embodiments, the subject is administered the composition once every four weeks or once every eight weeks. In some embodiments, the subject is administered the composition once every 4 weeks during a first period of administration (e.g., 8 weeks, 12 weeks, or 16 weeks), and subsequently administered the composition once every 8 weeks during a second period of administration (e.g., 16 weeks to the remainder of the subject's lifetime). In some embodiments, a composition comprising complexes for administration to a subject in the methods described herein further comprises complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, a method described herein comprises administering intravenously (e.g., via intravenous infusion) an effective amount of a composition comprising complexes (e.g., in an aqueous solution), wherein each complex comprises a structure of formula (I): [R¹]ₙ₁—R², wherein each R¹ of the complexes comprises a group of the formula (Ic):

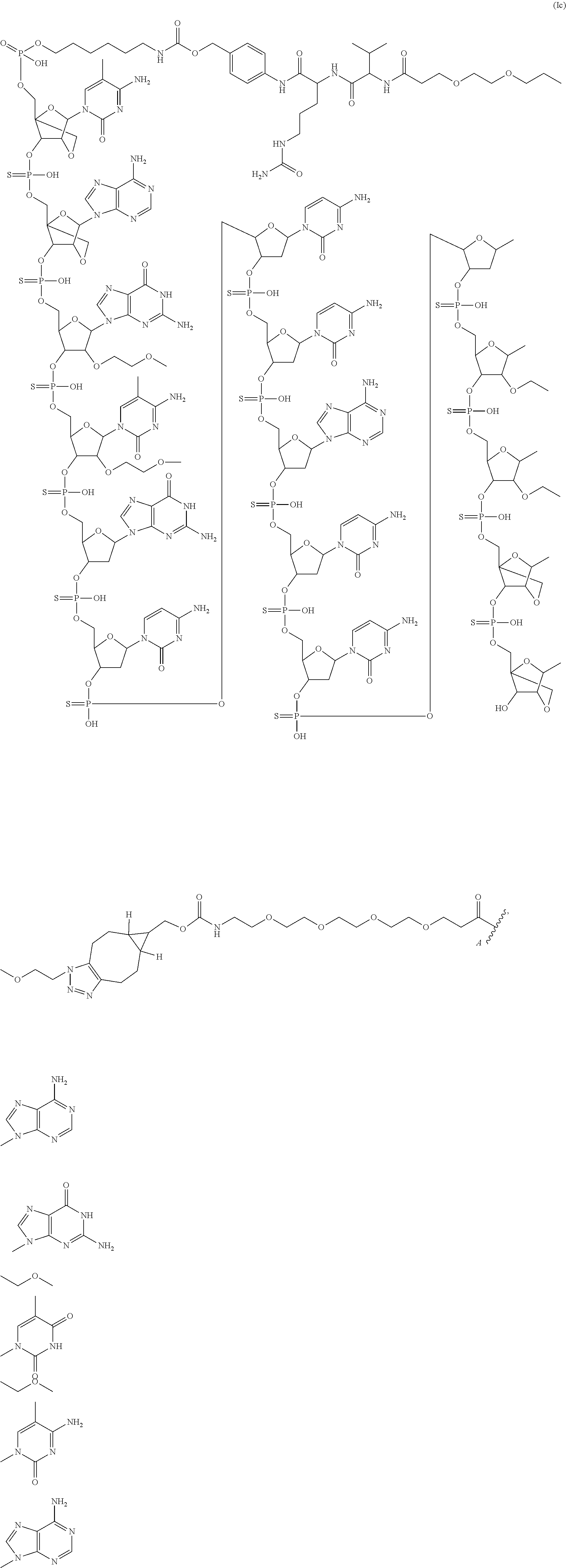

wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEO ID NO: 20;
wherein each R¹ is covalently linked to R² at attachment point A, optionally wherein each R¹ is covalently linked at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5;
wherein the effective amount of each administration provides to the subject 5 mg to 110 mg (e.g., 8 mg to 60 mg or 13 mg to 75 mg) of the anti-TfR1 antibody (e.g., Fab) of the complexes per kg of the subject. In some embodiments, the effective amount of each administration provides to the subject about 13 mg, about 25 mg, about 50 mg, or about 75 mg of the anti-TfR1 antibody (e.g., Fab) of the complexes per kg of the subject. In some embodiments, the effective amount of each administration provides to the subject about 5 mg, about 15 mg, about 30 mg, or about 60 mg of the anti-TfR1 antibody (e.g., Fab) of the complexes per kg of the subject. In some embodiments, the subject is administered the composition once every four weeks or once every eight weeks. In some embodiments, the subject is administered the composition once every 4 weeks during a first period of administration (e.g., 8 weeks, 12 weeks, or 16 weeks), and subsequently administered the composition once every 8 weeks during a second period of administration (e.g., 16 weeks to the remainder of the subject's lifetime). In some embodiments, a composition comprising complexes for administration to a subject in the methods described herein further comprises complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, a method described herein comprises administering intravenously (e.g., via intravenous infusion) an effective amount of a composition comprising complexes (e.g., in an aqueous solution), wherein each complex comprises a group of formula (Id):

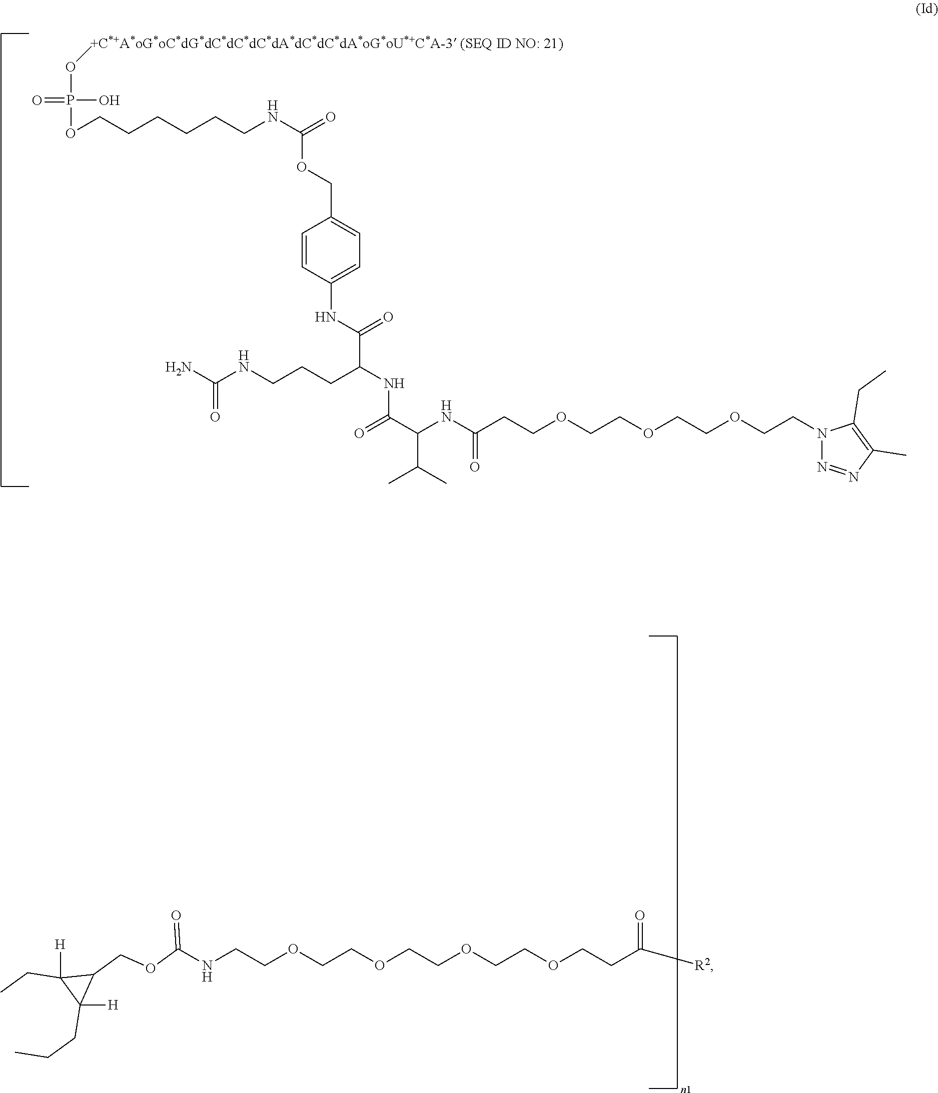

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21);
wherein R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20; and
wherein in each complex n1 is independently an integer of one or greater, optionally wherein the average value of n1 of the complexes of the composition is in the range of 1 to 5;
wherein the effective amount of each administration provides to the subject 5 mg to 110 mg (e.g., 8 mg to 60 mg or 13 mg to 75 mg) of the anti-TfR1 antibody (e.g., Fab) of the complexes per kg of the subject. In some embodiments, the effective amount of each administration provides to the subject about 13 mg, about 25 mg, about 50 mg, or about 75 mg of the anti-TfR1 antibody (e.g., Fab) of the complexes per kg of the subject. In some embodiments, the effective amount of each administration provides to the subject about 5 mg, about 15 mg, about 30 mg, or about 60 mg of the anti-TfR1 antibody (e.g., Fab) of the complexes per kg of the subject. In some embodiments, the subject is administered the composition once every four weeks or once every eight weeks. In some embodiments, the subject is administered the composition once every 4 weeks during a first period of administration (e.g., 8 weeks, 12 weeks, or 16 weeks), and subsequently administered the composition once every 8 weeks during a second period of administration (e.g., 16 weeks to the remainder of the subject's lifetime). In some embodiments, a composition comprising complexes for administration to a subject in the methods described herein further comprises complexes that comprise a structure of formula (I): [R¹]ₙ₁—R², wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, a method described herein comprises administering intravenously (e.g., via intravenous infusion) an effective amount of a composition comprising complexes (e.g., in an aqueous solution), wherein each complex comprises a group of the formula (Ia), (Ib), (Ic), or (Id), wherein the effective amount of each administration provides to the subject 0.5 mg to 20 mg (e.g., 1 mg to 14 mg or 0.5 mg to 12 mg) of the oligonucleotides of the complexes per kg of the subject. In some embodiments, the effective amount of each administration provides to the subject about 1.8 mg, about 3.4 mg, about 6.8 mg, or about 10.2 mg of the oligonucleotides of the complexes per kg of the subject. In some embodiments, the effective amount of each administration provides to the subject about 1.1 mg, about 2 mg, about 4.1 mg, or about 8.2 mg of the oligonucleotides of the complexes per kg of the subject. In some embodiments, the subject is administered the composition once every four weeks or once every eight weeks. In some embodiments, a composition comprising complexes (e.g., in aqueous solutions) for administration to a subject in the methods described herein further comprises complexes wherein n1 is 0. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, administration of a composition comprising an effective amount of complexes according to any one of the methods described herein reduces expression or activity of DMPK (e.g., reduces the level of a mutant or wild-type DMPK RNA, or reduces the activity of a DMPK gene product) in a subject (e.g., a subject having DM1). In some embodiments, administration of a composition comprising an effective amount of complexes according to any one of the methods described herein results in a reduction in expression or activity of DMPK (e.g., the level of a mutant or wild-type DMPK RNA, or the activity of a DMPK gene product) of at least 1% (e.g., at least 1%, at least 2%, at least 3%, at least 4%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, at least 50%, at least 60%, at least 70%, at least 80%, at least 90%) in the subject relative to a control. In some embodiments, the control is DMPK expression or activity level in a healthy subject (e.g., a subject that does not have myotonic dystrophy). In some embodiments, the control is DMPK expression or activity level in a subject that does not have a mutated DMPK RNA. In some embodiments, the control is DMPK expression or activity level in the subject prior to administration of the composition comprising the effective amount of complexes to the subject.

In some embodiments, any one of the methods described here may further comprise additional steps of administering compositions comprising the complexes described herein to the subject.

### EXAMPLES

### Example 1. In Vivo Tissue Distribution of Conjugates Containing Anti-TfR1 Fab Conjugated to a DMPK-Targeting Oligonucleotide in DM1 Mouse Model

Conjugates comprising an anti-TfR1 Fab conjugated to a DMPK-targeting oligonucleotide (ASO) were tested in a mouse model that expresses both human TfR1 and a human DMPK mutant that harbors expanded CUG repeats. The anti-TfR1 Fab used has the VH/VL sequences provided in Table 2. The Fab was covalently linked (through lysine conjugation) via a linker comprising a valine-citrulline sequence to a DMPK targeting oligonucleotide comprising a nucleobase sequence of SEQ ID NO: 21. The conjugate comprises a structure of formula (Id):

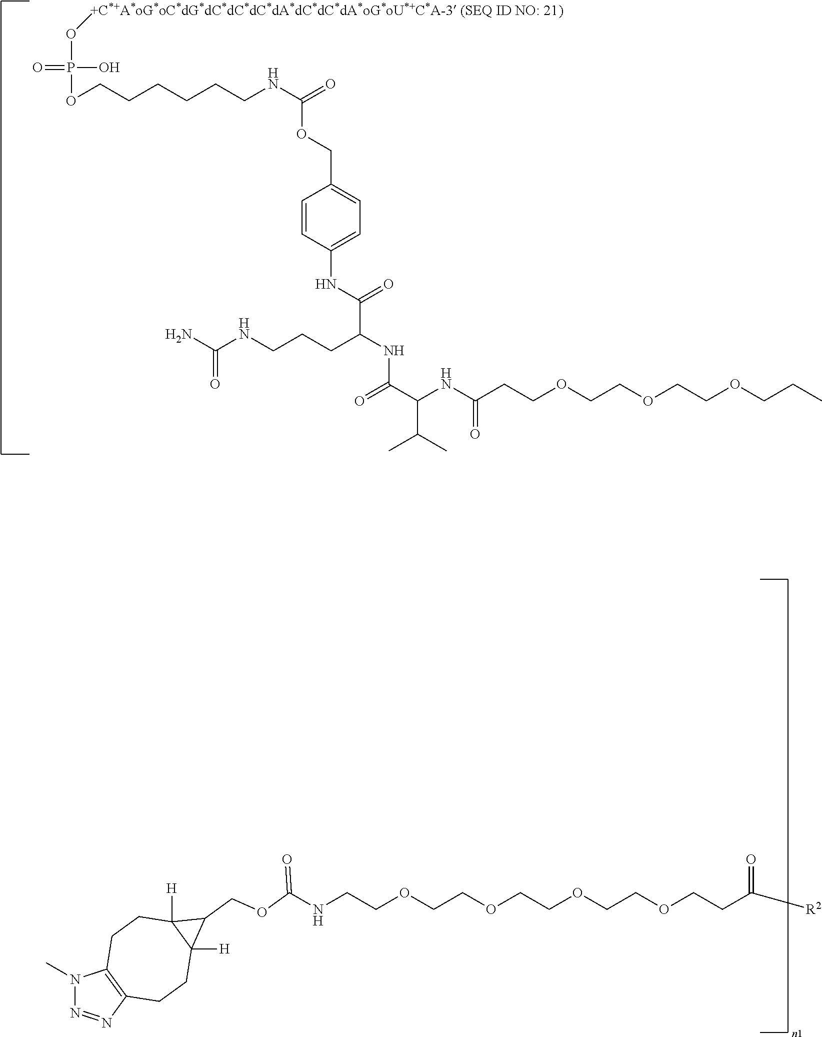

(Id), wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21), and in which R² comprises the anti-TfR1 antibody provided in Table 2, and wherein in each conjugate n1 is independently an integer of 1-3.

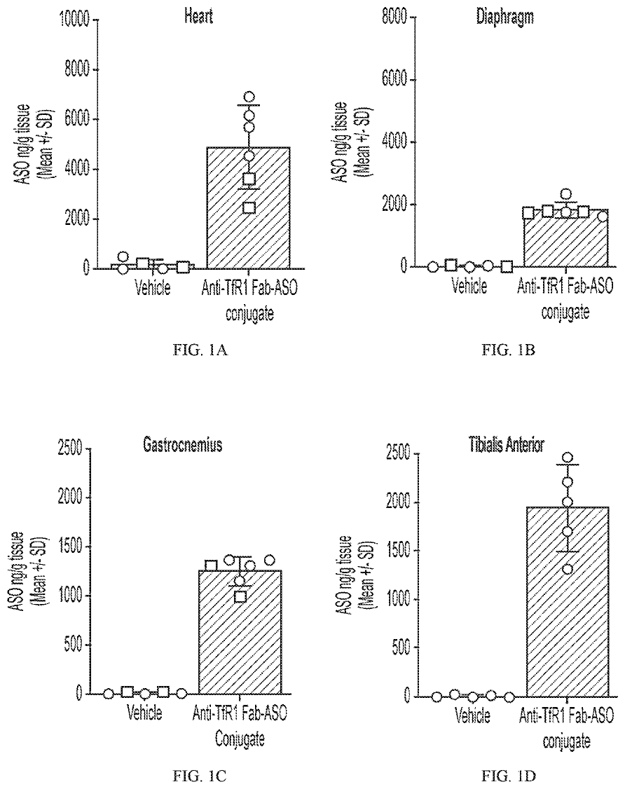

The conjugates were administered intravenously to the mice at day 0 and day 7, each time at a dose equivalent to 9.7 mg/kg of the ASO. The tissue exposure of the ASO was tested by hybridization ELISA (Burki et al., Nucleic Acid Ther. 2015 October; 25(5):275-84, incorporated herein by reference), and the levels of ASO in the tissue were graphed. FIGS. 1A, 1B, 1C, and 1D show the amount of ASO in the heart, diaphragm, gastrocnemius, or tibialis anterior, respectively, two weeks after the first injection. These results demonstrate that conjugates comprising an anti-TfR1 antibody (e.g., an anti-TfR1 Fab having the VH and VL sequences provided in Table 2) are capable of delivering an oligonucleotide (e.g., a DMPK-targeting ASO) to various muscle tissues following intravenous administration.

### Example 2. Sustained Knockdown of Toxic Human DMPK in hTfR1/DMSXL Homozygous Mice at 4 Weeks after Repeat Dosing of Anti-TfR1 Fab-ASO Conjugates

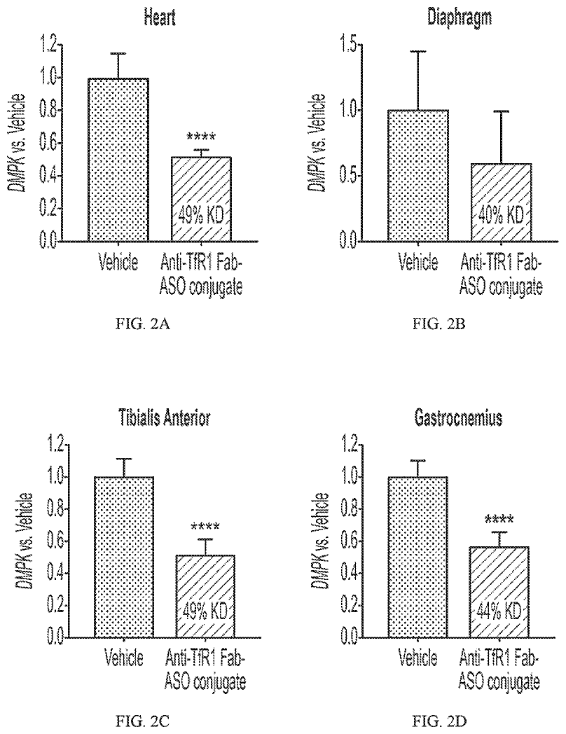

Conjugates (labeled in this Example as “Anti-TfR1 Fab-ASO conjugate”) as described in Example 1, containing an anti-TfR1 Fab covalently linked to a DMPK-targeting oligonucleotide (ASO) were tested in a mouse model that expresses both human TfR1 and two copies of a mutant human DMPK transgene that harbors expanded CUG repeats (hTfR1/DMSXL mice). Mice were administered either vehicle control (PBS) or 10 mg/kg ASO-equivalent dose of anti-TfR1 Fab-ASO conjugate at days 0 and 7. Mice were sacrificed at day 28 (four weeks following administration of the first dose of anti-TfR1 Fab-ASO conjugate), and tissues were collected. RNA was extracted and selected tissue samples were fixed, paraffin embedded and sectioned, then subjected to in situ hybridization. Reverse transcription-quantitative polymerase chain reaction (RT-qPCR) of the RNA samples was performed to measure human DMPK and mouse Ppib (peptidylprolyl isomerase) as an internal control. DMPK expression is shown in FIGS. 2A-2D as geometric means+/−standard deviation (n=6-9). Significance was assessed by Student's t-test (****P<0.0001).

FIG. 2A shows that anti-TfR1 Fab-ASO conjugate knocked down DMPK expression in heart by 49% relative to PBS-treated mice. FIG. 2B shows that anti-TfR1 Fab-ASO conjugate knocked down DMPK expression in diaphragm by 40% relative to PBS-treated mice. FIG. 2C shows that anti-TfR1 Fab-ASO conjugate knocked down DMPK expression in tibialis anterior by 49% relative to PBS-treated mice. FIG. 2D shows that anti-TfR1 Fab-ASO conjugate knocked down DMPK expression in gastrocnemius by 44% relative to PBS-treated mice.

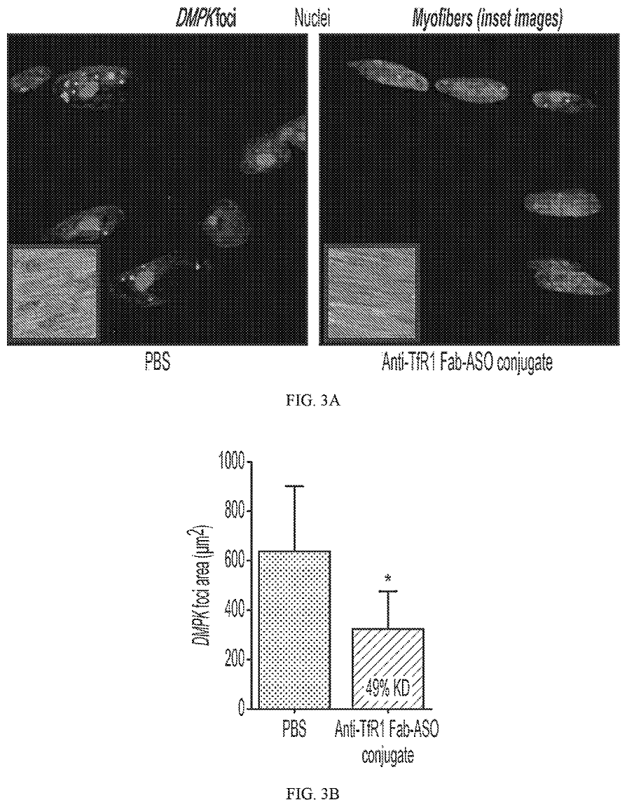

FIGS. 3A and 3B show that anti-TfR1 Fab-ASO conjugate reduced DMPK foci within nuclei of myofibers. FIG. 3A shows reduced DMPK foci by in situ hybridization, and FIG. 3B shows quantification of DMPK foci in fluorescent microscopy images, demonstrating the conjugate reduced foci area by 49%. Data are presented as mean+/−standard deviation (n=7). Significance was assessed by t-test (*P<0.05).

These results demonstrate that administration of anti-TfR1 Fab-ASO conjugate leads to robust, sustained knockdown of human toxic DMPK in cardiac and skeletal muscle.

### Example 3. Correction of Splicing Defects in hTfR1/DMSXL Homozygous Mice by Anti-TfR1 Fab-ASO Conjugates

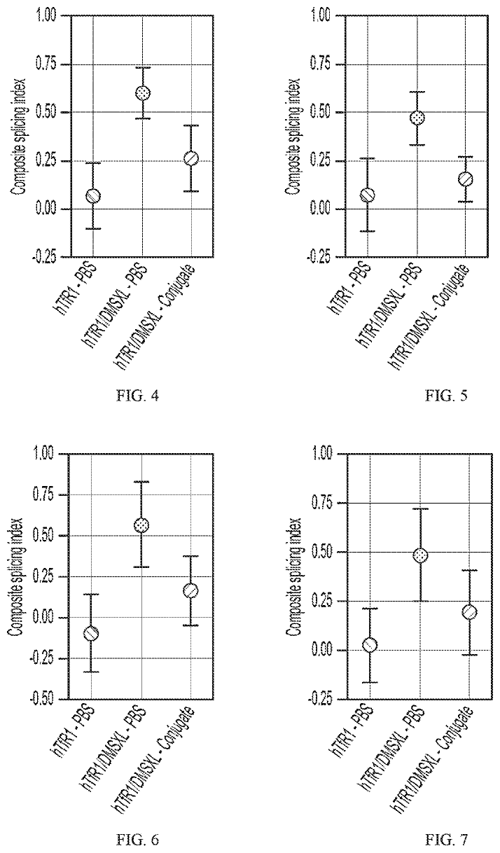

Conjugates (labeled in this Example as “Anti-TfR1 Fab-ASO conjugate”) as described in Example 1, containing an anti-TfR1 Fab covalently linked to a DMPK-targeting oligonucleotide (ASO) were tested in a mouse model (“hTfR1/DMSXL”) that expresses both human TfR1 and two copies of a mutant human DMPK transgene that harbors expanded CUG repeats. These mice are known to display splicing defects that are consistent with those observed in patients afflicted with DM1 (Huguet, et al. (2012) PLOS Genetics 8(11): e1003043). Mice were administered either vehicle control (“hTfR1/DMSXL—PBS”) or 10 mg/kg ASO-equivalent dose of anti-TfR1 Fab-ASO conjugate (“hTfR1/DMSXL—Conjugate”) on days 0 and 7. Mice expressing only the human TfR1 but not the mutant human DMPK transgene (hTfR1 mice) and treated with PBS (“hTfR1—PBS”) were used as another control to define the extent of the splicing phenotype in hTfR1/DMSXL mice and assess the magnitude of the effect of the conjugate on splicing. Mice were sacrificed on day 28 (four weeks following administration of the first dose of anti-TfR1 Fab-ASO conjugate), tissues were collected, and RNA was extracted. Reverse transcription-quantitative polymerase chain reaction (RT-qPCR) was performed to measure exon inclusion in a set of RNAs known to be mis-spliced during DM1 progression in humans and mice (Nakamori, et al. (2013) Ann. Neurol. 74(6): 862-872; Huguet, et al. (2012) PLOS Genetics 8(11): e1003043). Exon inclusion was calculated as normalized percent spliced in (PSI) for each splicing RNA marker, and composite splicing indices were calculated using the normalized PSI values from splicing markers in heart (FIG. 4), diaphragm (FIG. 5), tibialis anterior (FIG. 6), and gastrocnemius (FIG. 7). Composite splicing indices were calculated as previously described (Tanner M K, et al. (2021) Nucleic Acids Res. 49:2240-2254), and are shown as mean+/−standard deviation.

FIG. 4 shows that anti-TfR1 Fab-ASO conjugate corrected splicing in heart tissue of hTfR1/DMSXL mice, as demonstrated by composite splicing index data. The normalized PSI values used to generate the composite splicing index data showed correction of Mbnl2 exon 6 (E6) and Nfix E7 splicing in heart tissue of hTfR1/DMSXL mice by treatment with anti-TfR1 Fab-ASO conjugate, but did not show correction of Ldb3 E11 splicing. Composite splicing index data shown in FIG. 4 were based on Ldb3 E11, Mbnl2 E6, and Nfix E7 splicing data; Bin1E11, Dtna E12, Insr E11, and Mbnl2 E5 were not included because their normalized PSI values in heart tissue were not changed in hTfR1/DMSXL mice relative to hTfR1 mice under the experimental conditions tested.

FIG. 5 shows that anti-TfR1 Fab-ASO conjugate corrected splicing in diaphragm tissue of hTfR1/DMSXL mice, as demonstrated by composite splicing index data. The normalized PSI values used to generate the composite splicing index data showed correction of Bin1E11, Insr E11, Ldb3 E11 and Nfix E7 splicing in diaphragm tissue of hTfR1/DMSXL mice by treatment with anti-TfR1 Fab-ASO conjugate. Composite splicing index data shown in FIG. 5 were based on Bin1E11, Insr E11, Ldb3 E11 and Nfix E7 splicing data; Dtna E12, Mbnl2 E5, Mbnl2 E6, and Ttn E313 were not included because their normalized PSI values in diaphragm tissue were not changed in hTfR1/DMSXL mice relative to hTfR1 mice under the experimental conditions tested.

FIG. 6 shows that anti-TfR1 Fab-ASO conjugate corrected splicing in tibialis anterior tissue of hTfR1/DMSXL mice, as demonstrated by composite splicing index data. The normalized PSI values used to generate the composite splicing index data showed correction of Bin1E11, Ldb3 E11, and Nfix E7 splicing in tibialis anterior tissue of hTfR1/DMSXL mice by treatment with anti-TfR1 Fab-ASO conjugate, but did not show correction of Mbnl2 E6 splicing. Composite splicing index data shown in FIG. 6 were based on Bin1E11, Ldb3 E11, Mbnl2 E6, and Nfix E7 splicing data; Dtna E12, Insr E11, Mbnl2 E5, and Ttn E313 were not included because their normalized PSI values in tibialis anterior tissue were not changed in hTfR1/DMSXL mice relative to hTfR1 mice under the experimental conditions tested.

FIG. 7 shows that anti-TfR1 Fab-ASO conjugate corrected splicing in gastrocnemius tissue of hTfR1/DMSXL mice, as demonstrated by composite splicing index data. The normalized PSI values used to generate the composite splicing index data showed correction of Mbnl2 E6, Nfix E7, and Ttn E313 splicing in gastrocnemius tissue of hTfR1/DMSXL mice by treatment with anti-TfR1 Fab-ASO conjugate. Composite splicing index data shown in FIG. 7 were based on Mbnl2 E6, Nfix E7, and Ttn E313 splicing data; Bin1E11, Dtna E12, Insr E11, Ldb3 E11, and Mbnl2 E5 were not included because their normalized PSI values in gastrocnemius tissue were not changed in hTfR1/DMSXL mice relative to hTfR1 mice under the experimental conditions tested.

These results demonstrate that administration of anti-TfR1 Fab-ASO conjugate facilitates correction of DM1 splicing defects in cardiac and skeletal muscle.

### Example 4. DMPK Knockdown in Non-Human Primate and DM1 Patient Myotubes

Conjugates (labeled in this Example as “Anti-TfR1 Fab-ASO conjugate”) as described in Example 1, containing an anti-TfR1 Fab covalently linked to a DMPK-targeting oligonucleotide (ASO) were tested in human DM1 patient myotubes (32F cells) and in non-human primate (NHP) myotubes. The DM1 patient myotubes used express both a mutant DMPK mRNA containing 380 CUG repeats and a wild-type DMPK mRNA. The NHP myotubes used express only wild-type DMPK.

DM1 patient cells or NHP cells were seeded at a density of 50,000 cells per well in 96 well plates in growth medium and were allowed to recover overnight. The following day, the growth medium was changed to a low-serum differentiation medium and the cells were treated with conjugates at a concentration equivalent to 125 nM, 250 nM, or 500 nM ASO. The cells were incubated for ten days, then cDNA was synthesized using the Cells-to-Ct kit with crude cell lysates as the source of total RNA.

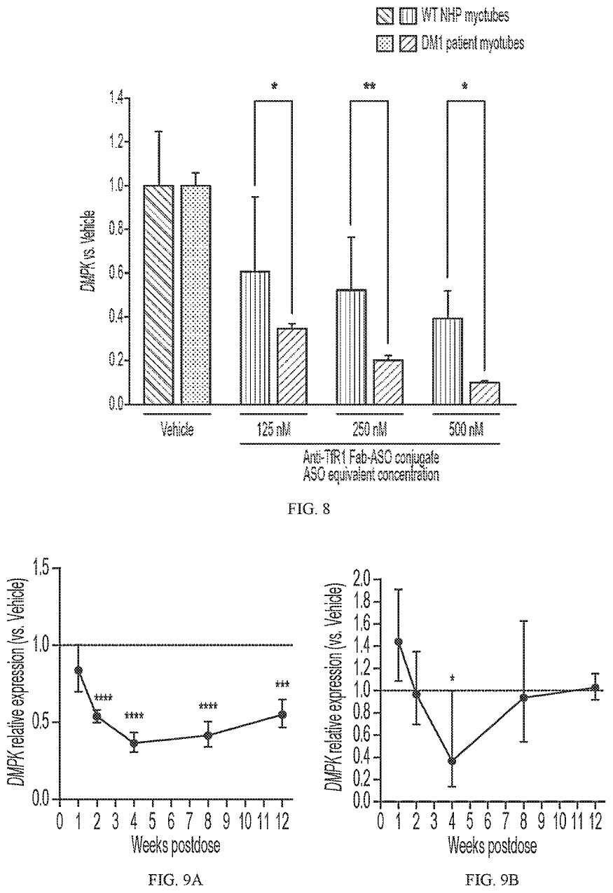

cDNA was used to assess total DMPK knockdown using Taqman PCR. The data was normalized to PPIB expression and the 2^{-ΔΔCt} method was used to determine DMPK knock down compared to a PBS-treated control (“Vehicle”). Data shown in FIG. 8 are presented as mean DMPK expression relative to species-matched vehicle control+standard deviation (n=4 replicates per condition).

The results show that the anti-TfR1 Fab-ASO conjugates achieved knockdown of DMPK expression in both normal NHP myotubes and DM1 patient myotubes, with greater knockdown of DMPK expression in DM1 patient cells (expressing both DMPK mRNA containing 380 CUG repeats and wild-type DMPK mRNA) compared to NHP cells (expressing only wild-type DMPK mRNA) when treated at physiologically relevant concentrations (FIG. 8). At an ASO-equivalent concentration of 125 nM, the conjugates achieved approximately 40% DMPK knockdown relative to vehicle-only control in NHP myotubes, and approximately 65% DMPK knockdown in DM1 patient myotubes. At an ASO-equivalent concentration of 250 nM, the conjugates achieved approximately 45% DMPK knockdown relative to vehicle-only control in NHP myotubes, and approximately 80% DMPK knockdown in DM1 patient myotubes. At an ASO-equivalent concentration of 500 nM, the conjugates achieved approximately 60% DMPK knockdown relative to vehicle-only control in NHP myotubes, and approximately 90% DMPK knockdown in DM1 patient myotubes.

These results indicate that conjugates containing anti-TfR1 Fab covalently linked to a DMPK-targeting oligonucleotide can achieve greater knockdown of DMPK in human myotubes expressing both wild-type DMPK mRNA and mutant DMPK mRNA (with expanded CUG repeats) relative to cynomolgus monkey myotubes expressing wild-type DMPK.

### Example 5. DMPK Knockdown in hTfR1/DMSXL Hemizygous Mice at 12 Weeks after Administration of Anti-TfR1 Fab-ASO Conjugates

Conjugates as described in Example 1, containing an anti-TfR1 Fab covalently linked to a DMPK-targeting oligonucleotide (ASO), were tested in a hTfR1/DMSXL hemizygous mouse model. Mutant human DMPK expression was monitored in various tissues for twelve weeks after intravenous administration via tail vein to the mice. Mice were administered conjugates at a 20 mg/kg ASO-equivalent dose. Heart, diaphragm, gastrocnemius, and tibialis anterior tissues were collected. Mutant human DMPK knockdown was measured by quantitative PCR (qPCR) for expression of DMPK mRNA. DMPK knockdown in treated mice was assayed 1 week (7 days), 2 weeks (14 days), 4 weeks (28 days), 8 weeks (56 days), and 12 weeks (84 days) after administration of the anti-TfR1 Fab-ASO conjugates.

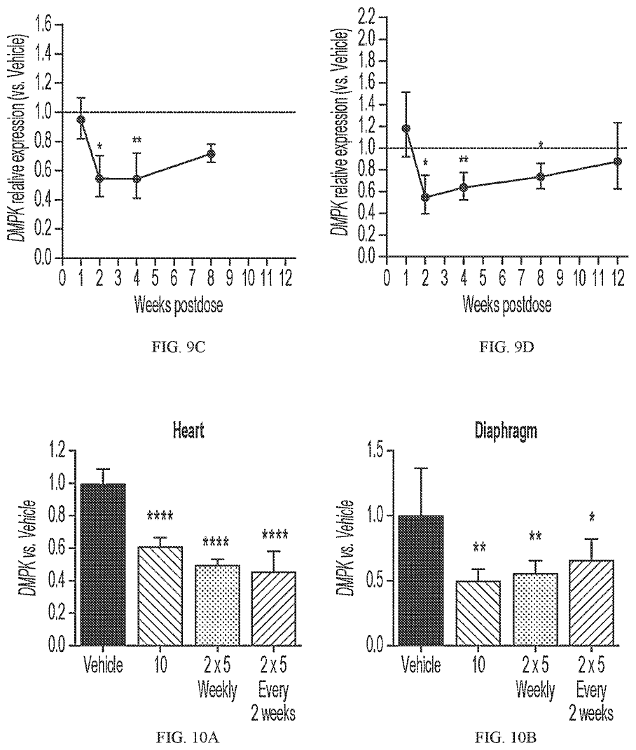

FIGS. 9A, 9B, 9C, and 9D show mutant human DMPK expression in heart, diaphragm, tibialis anterior, and gastrocnemius tissues, respectively, of treated mice, relative to control (PBS-treated) mice. Maximal knockdown of DMPK was generally attained 2-4 weeks after treatment, after which the DMPK knockdown diminished. Expression of mutant human DMPK in the heart remained suppressed up to 3 months after administration of the conjugates. Data are presented as means+/−standard deviation (n=5-12 replicates per tissue).

These data indicate the efficacy of administered anti-TfR1 Fab-ASO conjugates for at least two weeks after treatment.

### Example 6. DMPK Knockdown in hTfR1/DMSXL Hemizygous Mice after Administration of Anti-TfR1 Fab-ASO Conjugates

Conjugates as described in Example 1, containing an anti-TfR1 Fab covalently linked to a DMPK-targeting oligonucleotide (ASO), were further tested in a hTfR1/DMSXL hemizygous mouse model. DMPK expression in various tissues was assessed four weeks after treatment with the same dosage of anti-TfR1 Fab-ASO conjugates, administered either in a single dose or in two separate doses. Mice received either a single dose of the conjugates equivalent to 10 mg/kg of ASO, or two doses of the conjugates, each dose equivalent to 5 mg/kg of ASO. Mice receiving two 5 mg/kg doses received the doses either one week apart or two weeks apart. Four weeks (28 days) after administration of the final dose (either the 10 mg/kg dose or the second of two 5 mg/kg doses), DMPK knockdown and ASO levels were assessed in various muscle tissues.

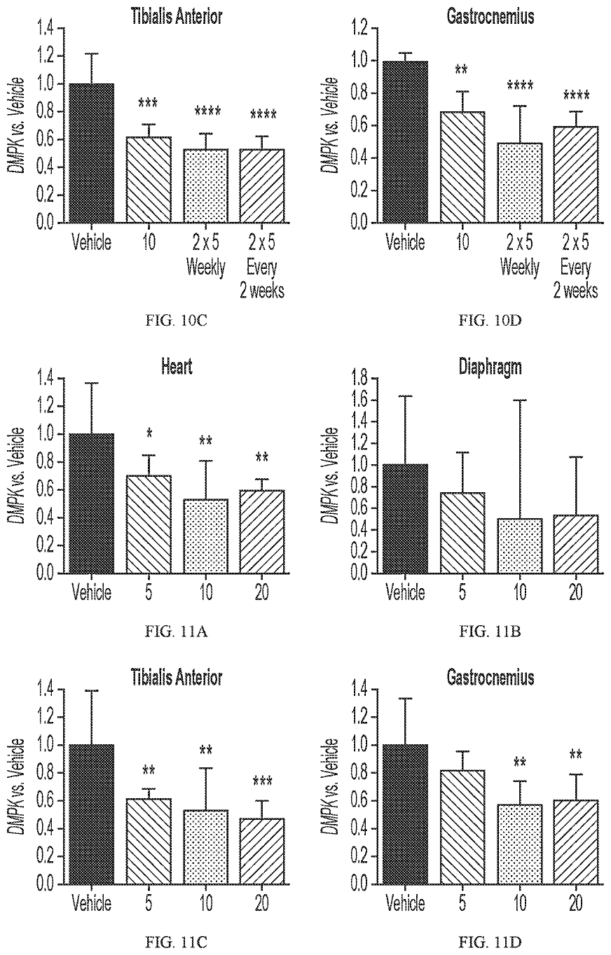

FIGS. 10A, 10B, 10C, and 10D show DMPK expression in heart, diaphragm, tibialis anterior, and gastrocnemius tissues, respectively, of treated mice, relative to control (vehicle-treated) mice. Mice treated with all dosing regimens sustained knockdown of toxic human DMPK for up to 4 weeks. Splitting the 10 mg/kg dose into two separate 5 mg/kg doses improved DMPK knockdown in some tested muscle tissues. The single 10 mg/kg dose resulted in 39% knockdown in heart, 50% knockdown in diaphragm, 38% knockdown in tibialis anterior, and 31% knockdown in gastrocnemius; the two 5 mg/kg doses administered one week apart resulted in 51% knockdown in heart, 45% knockdown in diaphragm, 46% knockdown in tibialis anterior, and 50% knockdown in gastrocnemius; and the two 5 mg/kg doses administered two weeks apart resulted in 55% knockdown in heart, 35% knockdown in diaphragm, 47% knockdown in tibialis anterior, and 40% knockdown in gastrocnemius, as measured 28 days after administration of the final dose of complexes. Data are presented as means+standard deviation (n=6 replicates per tissue). Significance was determined by a one-way ANOVA test.

These results indicate that anti-TfR1 Fab-ASO conjugates can be administered to subjects in doses that are temporally spaced to enhance the efficacy of DMPK knockdown without increasing the total dosage of the ASO administered to a subject.

### Example 7. DMPK Knockdown in hTfR1/DMSXL Hemizygous Mice after Administration of Anti-TfR1 Fab-ASO Conjugates

Conjugates as described in Example 1, containing an anti-TfR1 Fab covalently linked to a DMPK-targeting oligonucleotide (ASO), were further tested in a hTfR1/DMSXL hemizygous mouse model administered either in a single dose or in two doses spaced one week apart. DMPK expression was assessed in various tissues of the mice after treatment with the conjugates, relative to expression in the tissues of control (vehicle-treated) mice.

FIGS. 11A, 11B, 11C, and 11D show mutant human DMPK expression in heart, diaphragm, tibialis anterior, and gastrocnemius tissues, respectively, of mice treated with either one 5 mg/kg ASO-equivalent dose, one 10 mg/kg ASO-equivalent dose, or one 20 mg/kg ASO-equivalent dose of the conjugates. DMPK knockdown was assessed 28 days after administration of the conjugates. A dose-dependent trend of toxic human DMPK knockdown was observed, with approximately the same effect at the 10 mg/kg and 20 mg/kg doses tested at the 28-day timepoint. The 5 mg/kg dose resulted in 30% knockdown in heart, 26% knockdown in diaphragm, 39% knockdown in tibialis anterior, and 19% knockdown in gastrocnemius; the 10 mg/kg dose resulted in 46% knockdown in heart, 51% knockdown in diaphragm, 46% knockdown in tibialis anterior, and 42% knockdown in gastrocnemius; and the 20 mg/kg dose resulted in 41% knockdown in heart, 47% knockdown in diaphragm, 53% knockdown in tibialis anterior, and 40% knockdown in gastrocnemius, as measured 28 days after administration of the complexes. Data are presented as means+standard deviation. Significance was determined by a one-way ANOVA test.

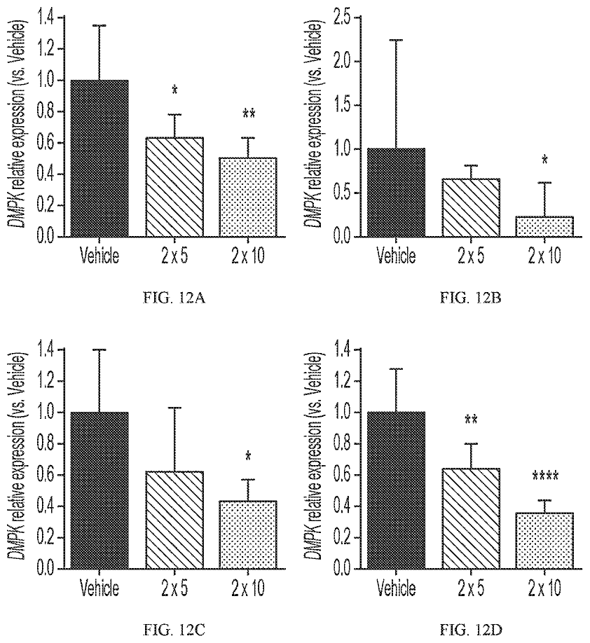

FIGS. 12A, 12B, 12C, and 12D show human mutant DMPK expression in heart, diaphragm, tibialis anterior, and gastrocnemius tissues, respectively, of mice treated with two 5 mg/kg ASO-equivalent doses (10 mg/kg total) or with two 10 mg/kg ASO-equivalent doses (20 mg/kg total) of the conjugates. The two 5 mg/kg ASO-equivalent doses and the two 10 mg/kg ASO-equivalent doses were administered one week apart. DMPK knockdown was assessed 28 days after administration of the first dose to mice (i.e., 21 days after the second dose was administered). A dose-dependent effect on DMPK knockdown was observed in all assayed tissues. Two 5 mg/kg doses of complexes resulted in 36% knockdown in heart, 34% knockdown in diaphragm, 36% knockdown in gastrocnemius, and 38% in tibialis anterior; two 10 mg/kg doses of complexes resulted in 51% knockdown in heart, 78% knockdown in diaphragm, 64% knockdown in gastrocnemius, and 57% in tibialis anterior, as measured 28 days after administration of the first dose of complexes. Data are presented as means+standard deviation (n=5-6 replicates per tissue). Significance was determined by Brown-Forsythe and Welch ANOVA test with Dunnett's T3 multiple comparisons test.

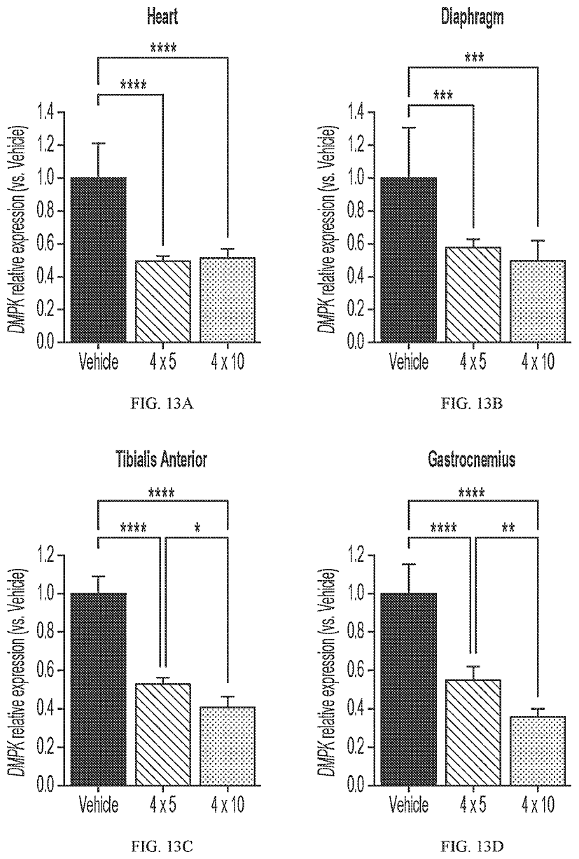

The anti-TfR1 Fab-ASO conjugates were also tested in hTfR1/DMSXL hemizygous mice after administration of four monthly doses of the conjugates to the mice. Conjugates were administered four 5 mg/kg ASO-equivalent doses or four 10 mg/kg ASO-equivalent doses of the conjugates, each administration 4 weeks apart (on days 0, 28, 56, and 84, respectively). DMPK knockdown was assessed in various tissues 112 days (16 weeks) after administration of the first dose to mice. FIGS. 13A, 13B, 13C, and 13D show human mutant DMPK expression in heart, diaphragm, tibialis anterior, and gastrocnemius tissues, respectively, of mice treated with the conjugates. DMPK knockdown was observed in all tissues tested, and both tibialis anterior and gastrocnemius showed statistically significant increase in knockdown in the mice administered four 10 mg/kg ASO-equivalent doses relative to those that received four 5 mg/kg doses. Four 5 mg/kg monthly doses of complexes resulted in 50% knockdown in heart, 43% knockdown in diaphragm, 45% knockdown in gastrocnemius, and 47% knockdown in tibialis anterior; and four 10 mg/kg monthly doses of complexes resulted in 48% knockdown in heart, 51% knockdown in diaphragm, 64% knockdown in gastrocnemius, and 59% knockdown in tibialis anterior, as measured 112 days after administration of the first dose of complexes. Data are presented as means+standard deviation (n=5-6 replicates per tissue). Significance was determined by ANOVA test followed by uncorrected Fisher's least significant difference test.

These results show the effect of various doses of anti-TfR1 Fab-ASO conjugates on expression of toxic human DMPK and suggest that efficacy of the conjugates may be enhanced by separating dosages of the conjugates.

### Example 8. DMPK Knockdown in Non-Human Primates Following Administration of Anti-TfR1 Fab-ASO Conjugates

Conjugates as described in Example 1, containing an anti-TfR1 Fab covalently linked to a DMPK-targeting oligonucleotide (ASO), were tested in a non-human primate (NHP) model (cynomolgus monkey; Macaca fascicularis) at dosages informed by the murine studies described in Examples 5-7. DMPK expression was assessed in various tissues after treatment with the conjugates, as measured using a quantitative PCR assay (qPCR) for expression of DMPK mRNA.

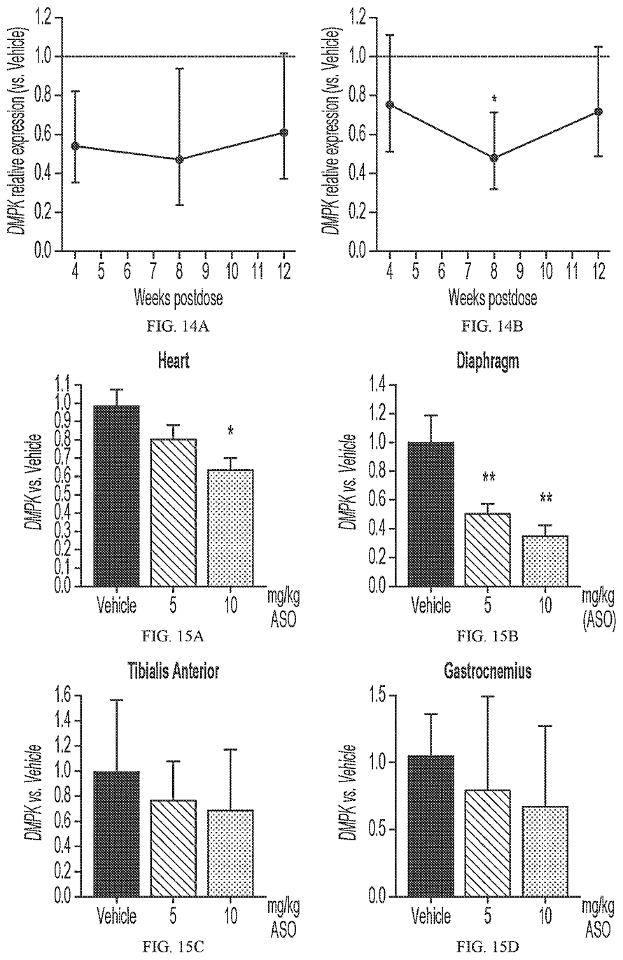

FIGS. 14A and 14B show DMPK expression in gastrocnemius and tibialis anterior tissues, respectively, of NHPs treated with an approximately 10 mg/kg ASO-equivalent dose of the conjugates, measured 4, 8, and 12 weeks after intravenous infusion of the conjugates. Expression is shown relative to that in vehicle-only control animals. Expression in animals administered the conjugates was decreased relative to the controls at each time point tested (relative expression in gastrocnemius of 0.54, 0.47, and 0.61 at 4, 8, and 12 weeks post-dose, respectively; relative expression in tibialis anterior of 0.75, 0.48, and 0.72 at 4, 8, and 12 weeks post-dose, respectively). Data are presented as means+/−standard deviation. Significance was determined by unpaired t-test.

FIGS. 15A, 15B, 15C, and 15D show DMPK expression in heart, diaphragm, tibialis anterior, and gastrocnemius tissues, respectively, of NHPs treated with either a 5 mg/kg or a 10 mg/kg ASO-equivalent dose of the conjugates, relative to control (vehicle-treated) animals. DMPK knockdown was assessed four weeks after administration of the conjugates. A dose-dependent effect of the conjugates on DMPK knockdown in cardiac and skeletal muscle was observed. The 5 mg/kg dose resulted in 21% knockdown in heart, 43% knockdown in diaphragm, 22% knockdown in tibialis anterior, and 25% knockdown in gastrocnemius; and the 10 mg/kg dose resulted in 38% knockdown in heart, 60% knockdown in diaphragm, 30% knockdown in tibialis anterior, and 35% knockdown in gastrocnemius at the assayed time. These results were consistent with the dosage effects observed in a hTfR1/DMSXL mouse model previously. Data are presented as means+standard deviation. Significance was determined by a one-way ANOVA test, using Dunnet's post-hoc analysis.

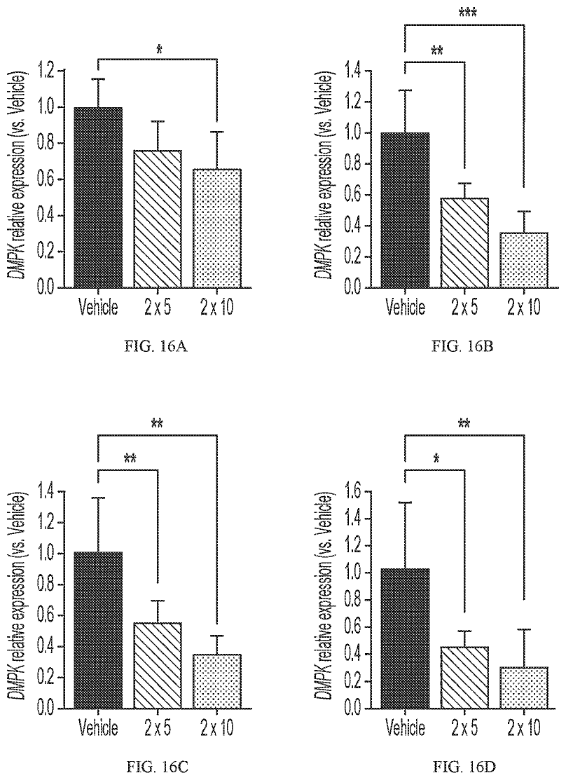

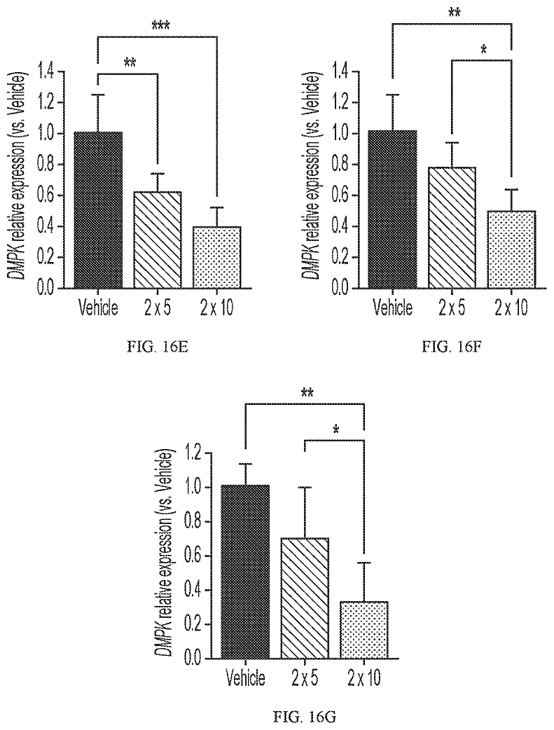

FIGS. 16A, 16B, 16C, 16D, 16E, 16F, and 16G show DMPK expression in heart, diaphragm, tibialis anterior, gastrocnemius, masseter, esophagus, and duodenum tissues, respectively, of NHPs treated with either two 5 mg/kg or two 10 mg/kg ASO-equivalent doses of the conjugates, with the two doses administered four weeks (28 days) apart. DMPK knockdown was assessed 8 weeks after administration of the first dose of the conjugates. Monthly administration of anti-TfR1 Fab-ASO conjugates led to sustained DMPK knockdown across all assayed tissues. Monthly administration of 10 mg/kg ASO-equivalent doses led to the greatest and most significant knockdown of WT DMPK in tissues relevant for DM1 pathology. Two 5 mg/kg ASO-equivalent doses of complexes resulted in 24% knockdown in heart, 41% knockdown in diaphragm, 56% knockdown in gastrocnemius, and 45% knockdown in tibialis anterior, 39% knockdown in masseter, 21% knockdown in esophagus, and 28% knockdown in duodenum; two 10 mg/kg ASO-equivalent doses of complexes resulted in 34% knockdown in heart, 64% knockdown in diaphragm, 70% knockdown in gastrocnemius, and 65% knockdown in tibialis anterior, 60% knockdown in masseter, 49% knockdown in esophagus, and 67% knockdown in duodenum, as measured 8 weeks after administration of the first dose. Data are presented as means+standard deviation. Significance was determined by a one-way ANOVA followed by an uncorrected Fisher's Least Significant Difference (LSD) test.

These results show that the anti-TfR1 Fab-ASO conjugates are effective in a NHP model. These results further indicate the prolonged efficacy of the conjugates when administered monthly and are consistent with results previously obtained in a mouse model that recapitulates the pathological development of DM1.

### Additional Embodiments

1. A method of reducing DMPK expression in a subject, comprising administering to the subject a composition comprising an effective amount of complexes comprising an anti-transferrin receptor 1 (TfR1) antibody covalently linked to one or more oligonucleotides, wherein the effective amount provides to the subject 5 mg to 110 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotides of the complexes comprise the nucleobase sequence of CAGCGCCCACCAGUCA (SEQ ID NO: 21).

2. A method of treating myotonic dystrophy in a subject, comprising administering to the subject a composition comprising an effective amount of complexes comprising an anti-transferrin receptor 1 (TfR1) antibody covalently linked to one or more oligonucleotides, wherein the effective amount provides to the subject 5 mg to 110 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotides of the complexes comprise the nucleobase sequence of CAGCGCCCACCAGUCA (SEQ ID NO: 21).

3. A method of reducing DMPK expression in a subject, comprising administering to the subject a composition comprising an effective amount of complexes comprising an anti-transferrin receptor 1 (TfR1) antibody covalently linked to one or more oligonucleotides, wherein the effective amount provides to the subject 1 mg to 12 mg of the oligonucleotides of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotides of the complexes comprise the nucleobase sequence of CAGCGCCCACCAGUCA (SEQ ID NO: 21).

4. A method of treating myotonic dystrophy in a subject, comprising administering to the subject a composition comprising an effective amount of complexes comprising an anti-transferrin receptor 1 (TfR1) antibody covalently linked to one or more oligonucleotides, wherein the effective amount provides to the subject 1 mg to 12 mg of the oligonucleotides of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotides of the complexes comprise the nucleobase sequence of CAGCGCCCACCAGUCA (SEQ ID NO: 21).

5. The method of any one of embodiments 1-4, wherein the oligonucleotides of the complexes comprise a 5′-X—Y—Z-3′ configuration, wherein X and Z are flanking regions comprising one or more modified nucleosides and Y is a gap region comprising one or more 2′-deoxyribonucleosides.

6. The method of any one of embodiments 1-5, wherein each complex comprises a structure of formula (I): [R¹]ₙ₁—R², wherein:
each R¹ comprises a group of the formula (Ib):

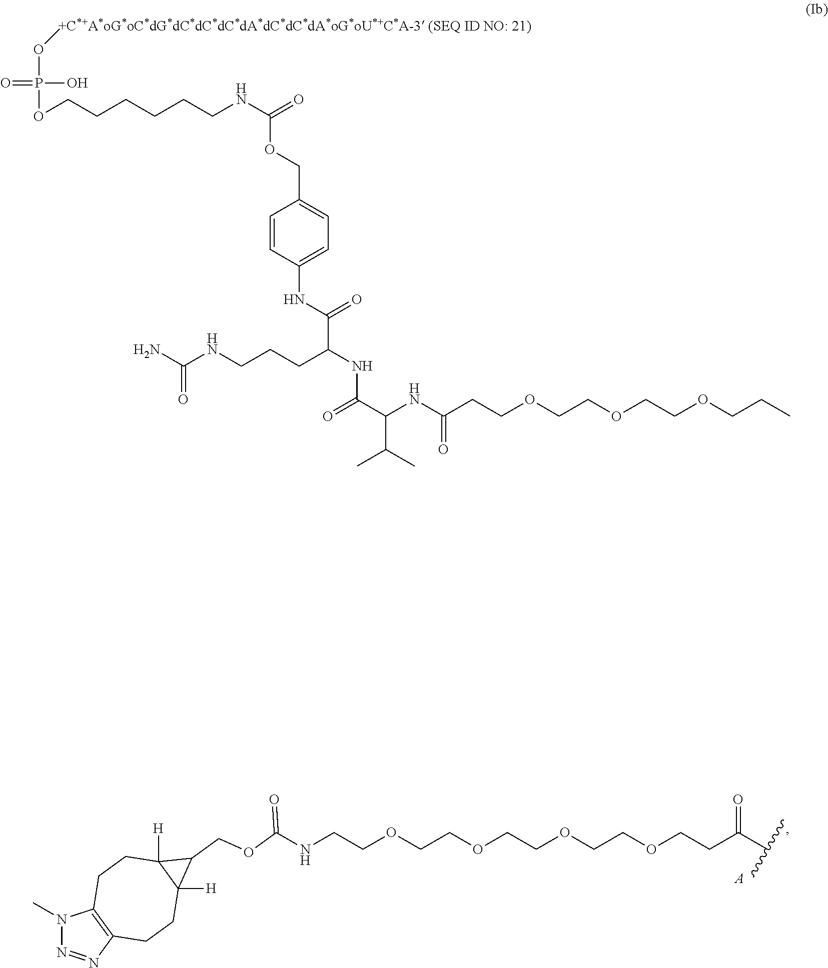

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5.

7. The method of any one of embodiments 1-5, wherein each complex comprises a structure of formula (I): [R¹]ₙ₁—R², wherein:
each R¹ comprises a group of the formula (Ic):

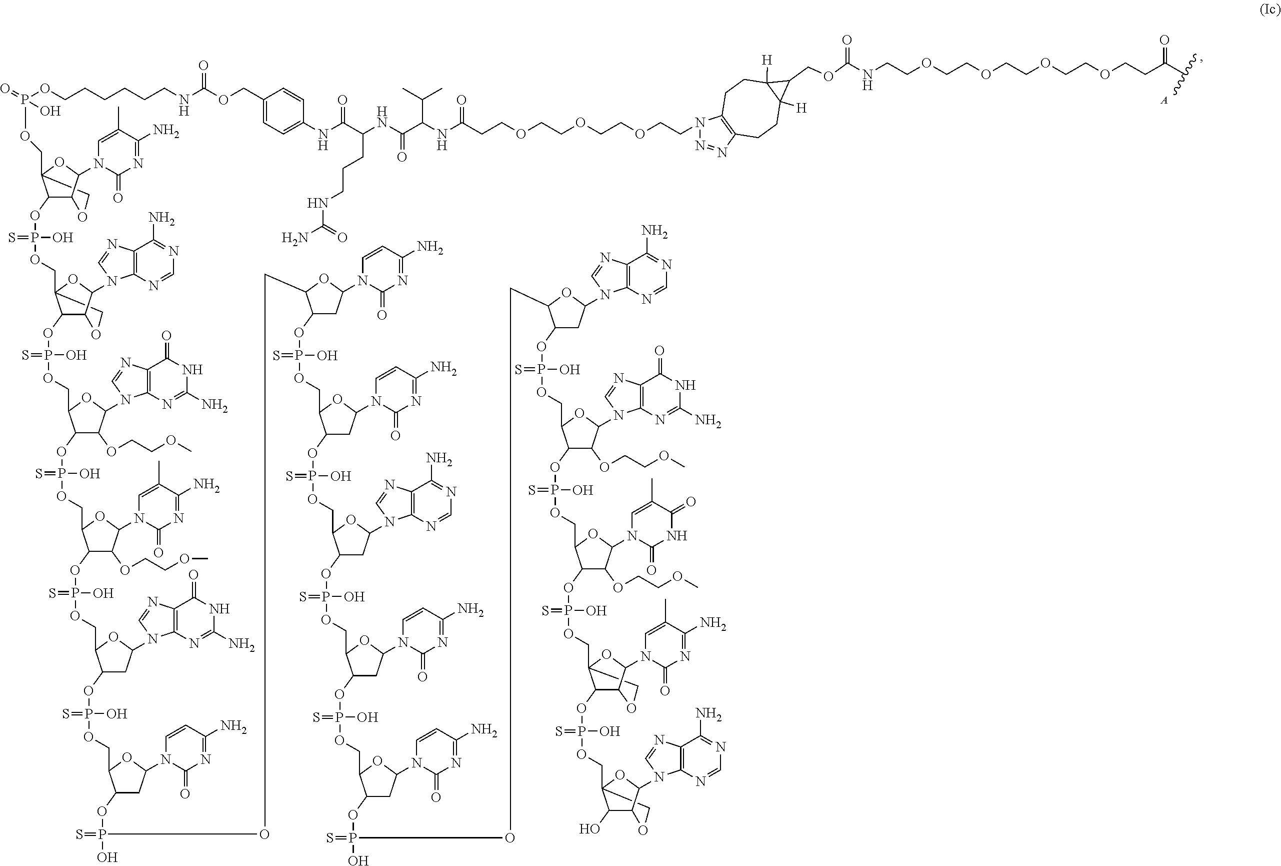

wherein R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5.

8. The method of any one of embodiments 1-7, wherein the anti-TfR1 antibody is a Fab fragment.

9. The method of any one of embodiments 1-8, wherein the anti-TfR1 antibody comprises a heavy chain variable region (VH) comprising the amino acid sequence of SEQ ID NO: 17 and a light chain variable region (VL) comprising the amino acid sequence of SEQ ID NO: 18.

10. The method of any one of embodiments 1-9, wherein the anti-TfR1 antibody comprises a heavy chain comprising the amino acid sequence of SEQ ID NO: 19 and a light chain comprising the amino acid sequence of SEQ ID NO: 20.

11. The method of any one of embodiments 1-10, wherein the administration occurs one or more times.

12. The method of embodiment 11, wherein the effective amount of each administration provides to the subject 10 mg to 110 mg of the anti-TfR1 antibodies of the complexes per kg of the subject.

13. The method of embodiment 11, wherein the effective amount of each administration provides to the subject 5 mg to 90 mg of the anti-TfR1 antibodies of the complexes per kg of the subject.

14. The method of embodiment 11, wherein the effective amount of each administration provides to the subject 10 mg to 20 mg of the anti-TfR1 antibodies of the complexes per kg of the subject, optionally wherein the effective amount of each administration provides to the subject 13 mg of the anti-TfR1 antibodies of the complexes per kg of the subject.

15. The method of embodiment 11, wherein the effective amount of each administration provides to the subject 18 mg to 36 mg of the anti-TfR1 antibodies of the complexes per kg of the subject, optionally wherein the effective amount of each administration provides to the subject 25 mg of the anti-TfR1 antibodies of the complexes per kg of the subject.

16. The method of embodiment 11, wherein the effective amount of each administration provides to the subject 36 mg to 72 mg of the anti-TfR1 antibodies of the complexes per kg of the subject, optionally wherein the effective amount of each administration provides to the subject 50 mg of the anti-TfR1 antibodies of the complexes per kg of the subject.

17. The method of embodiment 11, wherein the effective amount of each administration provides to the subject 55 mg to 110 mg of the anti-TfR1 antibodies of the complexes per kg of the subject, optionally wherein the effective amount of each administration provides to the subject 75 mg of the anti-TfR1 antibodies of the complexes per kg of the subject.

18. The method of embodiment 11, wherein the effective amount of each administration provides to the subject 6 mg to 12 mg of the anti-TfR1 antibodies of the complexes per kg of the subject, optionally wherein the effective amount of each administration provides to the subject 8 mg of the anti-TfR1 antibodies of the complexes per kg of the subject.

19. The method of embodiment 11, wherein the effective amount of each administration provides to the subject 11 mg to 22 mg of the anti-TfR1 antibodies of the complexes per kg of the subject, optionally wherein the effective amount of each administration provides to the subject 15 mg of the anti-TfR1 antibodies of the complexes per kg of the subject.

20. The method of embodiment 11, wherein the effective amount of each administration provides to the subject 22 mg to 44 mg of the anti-TfR1 antibodies of the complexes per kg of the subject, optionally wherein the effective amount of each administration provides to the subject 30 mg of the anti-TfR1 antibodies of the complexes per kg of the subject.

21. The method of embodiment 11, wherein the effective amount of each administration provides to the subject 44 mg to 88 mg of the anti-TfR1 antibodies of the complexes per kg of the subject, optionally wherein the effective amount of each administration provides to the subject 60 mg of the anti-TfR1 antibodies of the complexes per kg of the subject.

22. The method of embodiment 11, wherein the effective amount of each administration provides to the subject 1 mg to 14 mg of the oligonucleotides of the complexes per kg of the subject.

23. The method of embodiment 11, wherein the effective amount of each administration provides to the subject 0.5 mg to 12 mg of the oligonucleotides of the complexes per kg of the subject.

24. The method of embodiment 11, wherein the effective amount of each administration provides to the subject 1.2 mg to 2.4 mg of the oligonucleotides of the complexes per kg of the subject, optionally wherein the effective amount of each administration provides to the subject 1.8 mg of the oligonucleotides of the complexes per kg of the subject.

25. The method of embodiment 11, wherein the effective amount of each administration provides to the subject 2.2 mg to 4.5 mg of the oligonucleotides of the complexes per kg of the subject, optionally wherein the effective amount of each administration provides to the subject 3.4 mg of the oligonucleotides of the complexes per kg of the subject.

26. The method of embodiment 11, wherein the effective amount of each administration provides to the subject 4.5 mg to 9 mg of the oligonucleotides of the complexes per kg of the subject, optionally wherein the effective amount of each administration provides to the subject 6.8 mg of the oligonucleotides of the complexes per kg of the subject.

27. The method of embodiment 11, wherein the effective amount of each administration provides to the subject 7 mg to 14 mg of the oligonucleotides of the complexes per kg of the subject, optionally wherein the effective amount of each administration provides to the subject 10.2 mg of the oligonucleotides of the complexes per kg of the subject.

28. The method of embodiment 11, wherein the effective amount of each administration provides to the subject 0.7 mg to 1.5 mg of the oligonucleotides of the complexes per kg of the subject, optionally wherein the effective amount of each administration provides to the subject 1.1 mg of the oligonucleotides of the complexes per kg of the subject.

29. The method of embodiment 11, wherein the effective amount of each administration provides to the subject 1.4 mg to 2.7 mg of the oligonucleotides of the complexes per kg of the subject, optionally wherein the effective amount of each administration provides to the subject 2 mg of the oligonucleotides of the complexes per kg of the subject.

30. The method of embodiment 11, wherein the effective amount of each administration provides to the subject 2.7 mg to 5.4 mg of the oligonucleotides of the complexes per kg of the subject, optionally wherein the effective amount of each administration provides to the subject 4.1 mg of the oligonucleotides of the complexes per kg of the subject.

31. The method of embodiment 11, wherein the effective amount of each administration provides to the subject 5 mg to 11 mg of the oligonucleotides of the complexes per kg of the subject, optionally wherein the effective amount of each administration provides to the subject 8.2 mg of the oligonucleotides of the complexes per kg of the subject.

32. The method of embodiment 11, wherein the effective amount of each administration provides to the subject 5 mg to 8 mg of the oligonucleotides of the complexes per kg of the subject, optionally wherein the effective amount of each administration provides to the subject 5 mg of the oligonucleotides of the complexes per kg of the subject.

33. The method of any one of embodiments 1-32, wherein during a period of administration, the composition is administered once every 4 weeks.

34. The method of any one of embodiments 1-32, wherein during a period of administration, the composition is administered once every 8 weeks.

35. The method of any one of embodiments 1-32, wherein during a period of administration, the composition is administered once every 12 weeks.

36. The method of any one of embodiments 33-35, wherein the period of administration is less than 10 years.

37. The method of any one of embodiments 33-35, wherein the period of administration is the remainder of the subject's lifetime.

38. The method of any one of embodiments 1-32, wherein the composition is administered once every 4 weeks during a first period of administration, and subsequently administered once every 8 weeks during a second period of administration.

39. The method of embodiment 38, wherein the first period of administration is 8-16 weeks, and/or wherein the second period of administration is 16 weeks to the remainder of the subject's lifetime.

40. The method of any one of embodiments 1-39, wherein the composition is in the form of an aqueous solution and further comprises tris(hydroxymethyl)aminomethane and sucrose.

41. The method of embodiment 40, wherein the tris(hydroxymethyl)aminomethane is present in the aqueous solution at a concentration of 25 mM, the sucrose is present in the aqueous solution at a concentration of 10 w/v %, and the aqueous solution is at a pH of 7.5.

42. The method of embodiment 40 or embodiment 41, wherein the complexes are present in the composition at a concentration in the range of 10 mg/mL to 50 mg/mL.

43. The method of any one of embodiments 1-42, wherein the administering reduces DMPK expression in a muscle cell of the subject, optionally wherein reducing DMPK expression comprises reducing the amount of DMPK RNA in the muscle cell, optionally wherein the DMPK RNA amount is reduced in the nucleus of the muscle cell.

44. The method of embodiment 43, wherein reducing DMPK expression in the muscle cell comprises reducing the amount of DMPK protein in the muscle cell.

45. The method of any one of embodiments 1-44, wherein the subject has a mutant DMPK allele comprising disease-associated CTG repeats.

46. The method of embodiment 45, wherein the administering results in a reduction of DMPK mRNA in a muscle cell in the subject by at least 30%, optionally wherein the administering results in a reduction of a DMPK mRNA in the nucleus of a muscle cell in the subject.

47. The method of any one of embodiments 1-46, wherein the subject is human.

48. The method of any one of embodiments 1-46, wherein the subject is a rodent. 49. The method of any one of embodiments 1-46, wherein the subject is a non-human primate.

50. The method of any one of embodiments 1-49, wherein the complex is administered systemically, optionally wherein the complex is administered intravenously, further optionally wherein the complex is administered by infusion.

51. A method of reducing DMPK expression or treating myotonic dystrophy in a subject, comprising administering intravenously an effective amount of a composition comprising complexes comprising a structure of formula (I): [R¹]ₙ₁—R², wherein:
each R¹ comprises a group of the formula (Ia):

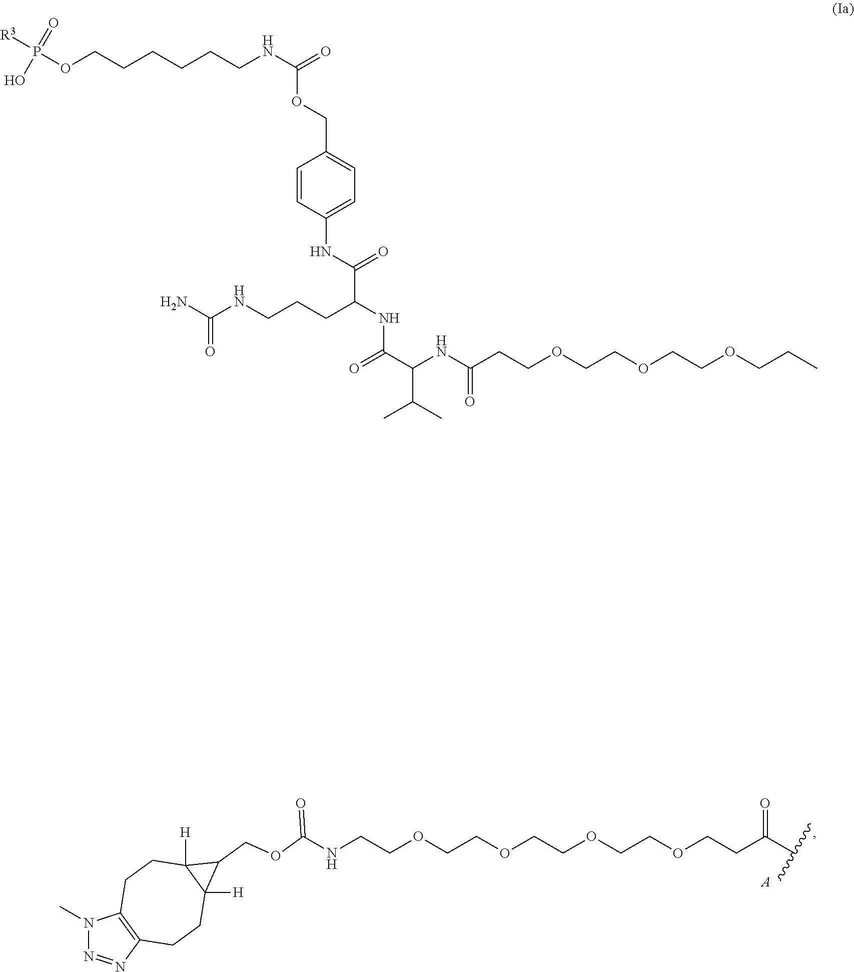

in which R³ comprises an oligonucleotide comprising a nucleobase sequence of CAGCGCCCACCAGUCA (SEQ ID NO: 21) and comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 5 mg to 110 mg of the anti-TfR1 antibody of the complexes per kg of the subject, optionally wherein the effective amount provides to the subject 8 mg to 60 mg or 13 mg to 75 mg of the anti-TfR1 antibody of the complexes per kg of the subject.

52. A method of reducing DMPK expression or treating myotonic dystrophy in a subject, comprising administering intravenously an effective amount of a composition comprising complexes comprising a structure of formula (I): [R¹]ₙ₁—R², wherein:
each R¹ comprises a group of the formula (Ib):

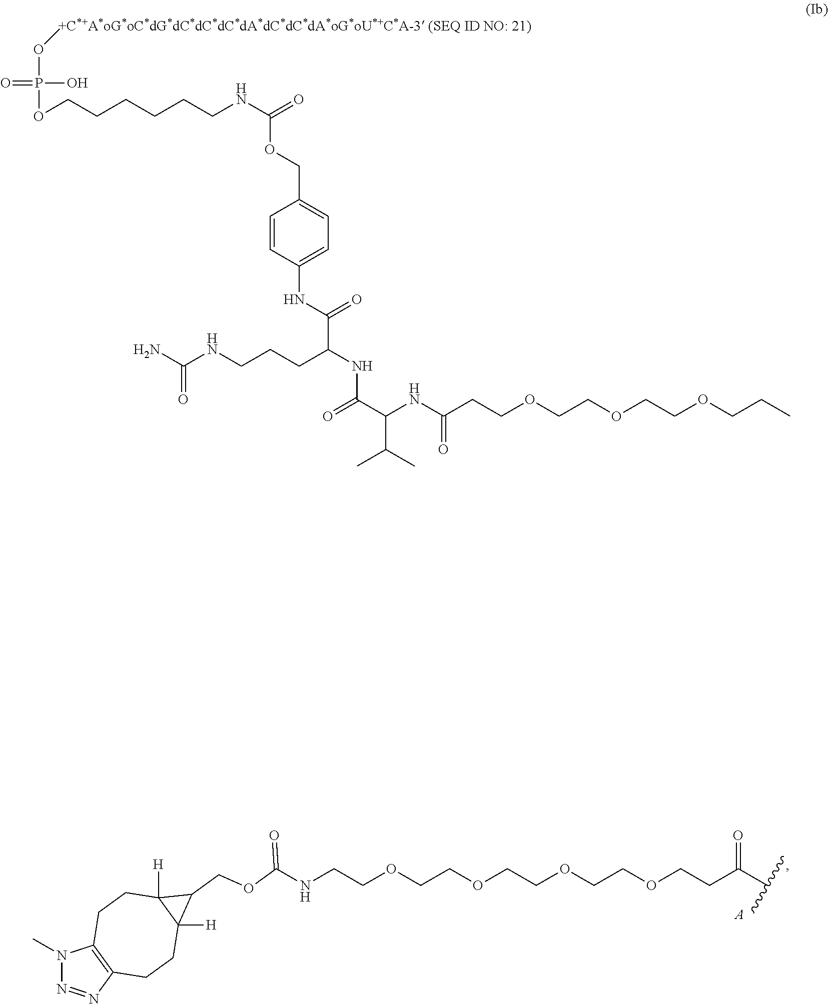

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 5 mg to 110 mg of the anti-TfR1 antibody of the complexes per kg of the subject, optionally wherein the effective amount provides to the subject 8 mg to 60 mg or 13 mg to 75 mg of the anti-TfR1 antibody of the complexes per kg of the subject.

53. A method of reducing DMPK expression or treating myotonic dystrophy in a subject, comprising administering intravenously an effective amount of a composition comprising complexes comprising a structure of formula (I): [R¹]ₙ₁—R², wherein:
each R¹ comprises a group of the formula (Ic):

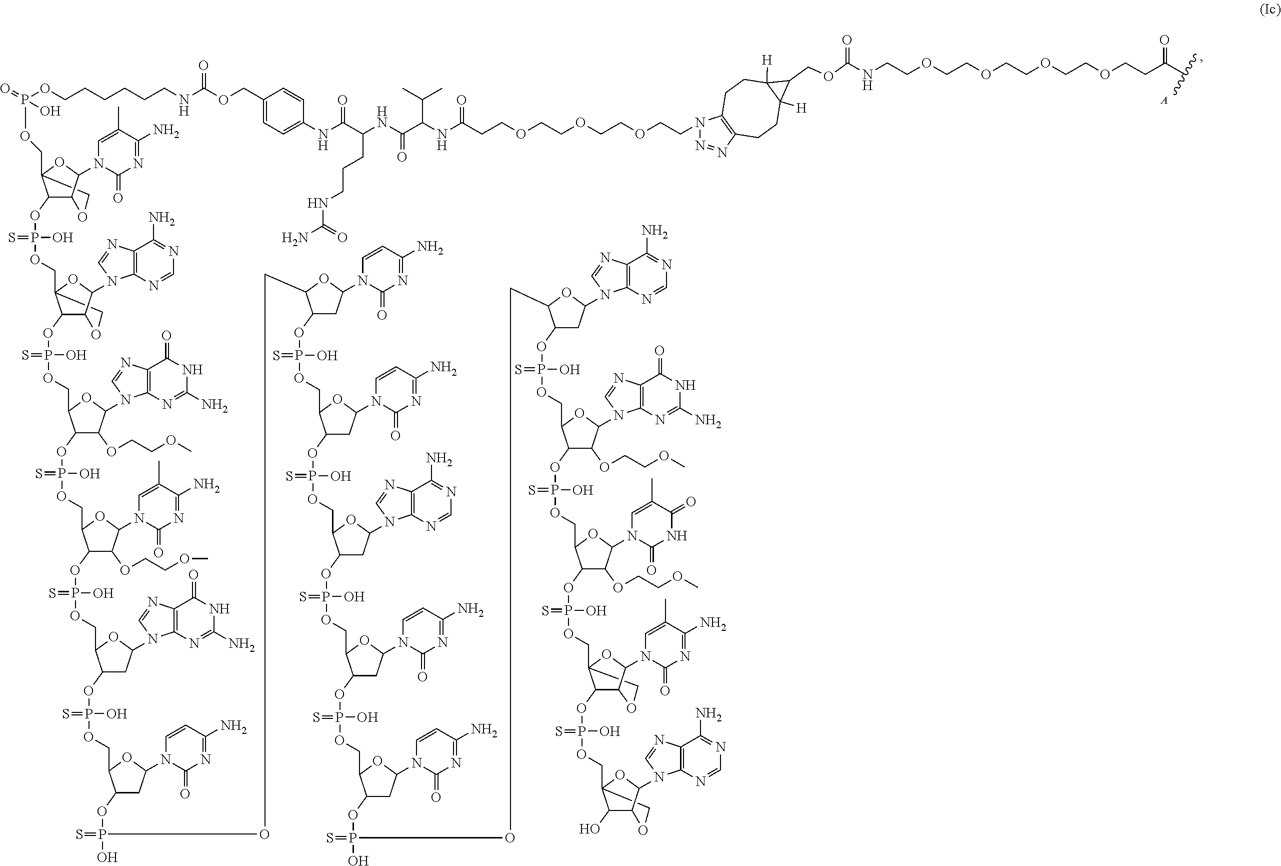

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 5 mg to 110 mg of the anti-TfR1 antibody of the complexes per kg of the subject, optionally wherein the effective amount provides to the subject 8 mg to 60 mg or 13 mg to 75 mg of the anti-TfR1 antibody of the complexes per kg of the subject.

54. A method of reducing DMPK expression or treating myotonic dystrophy in a subject, comprising administering intravenously an effective amount of a composition comprising complexes comprising a structure of the formula (Id):

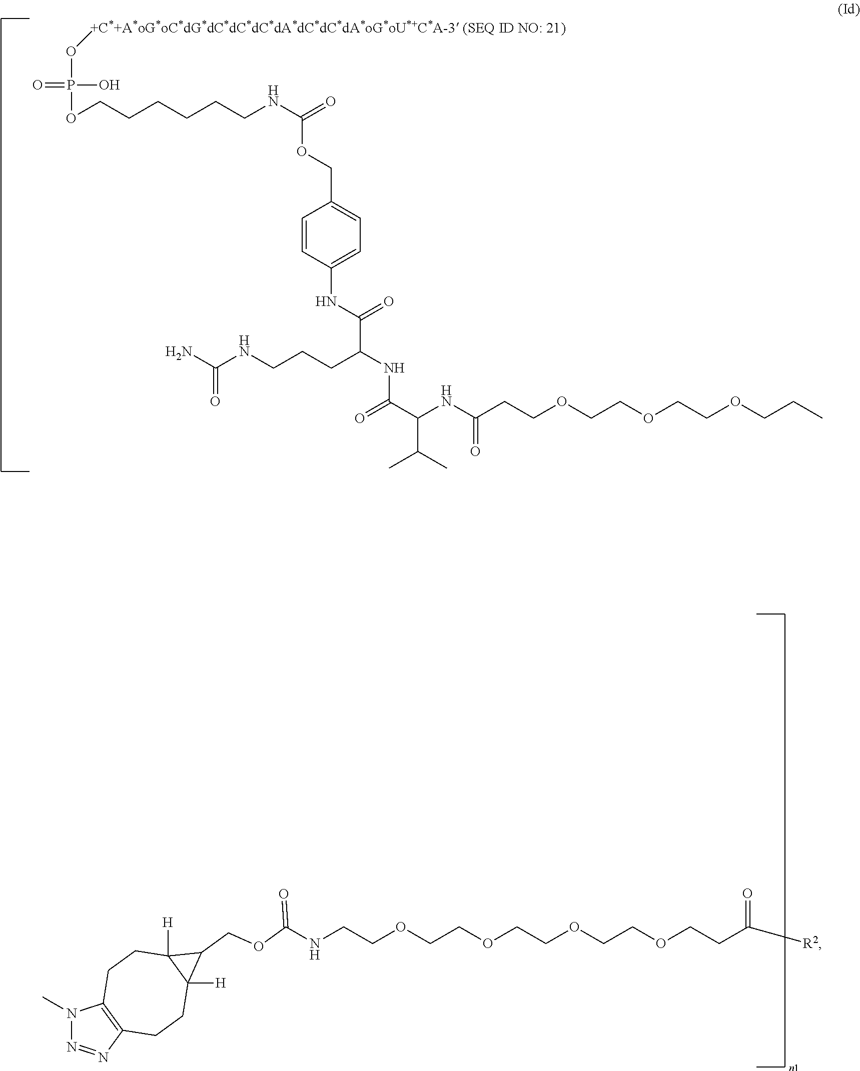

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 5 mg to 110 mg of the anti-TfR1 antibody of the complexes per kg of the subject, optionally wherein the effective amount provides to the subject 8 mg to 60 mg or 13 mg to 75 mg of the anti-TfR1 antibody of the complexes per kg of the subject.

55. A method of reducing DMPK expression or treating myotonic dystrophy in a subject, comprising administering intravenously an effective amount of a composition comprising complexes comprising a structure of formula (I): [R¹]ₙ₁—R², wherein:
each R¹ comprises a group of the formula (Ia):

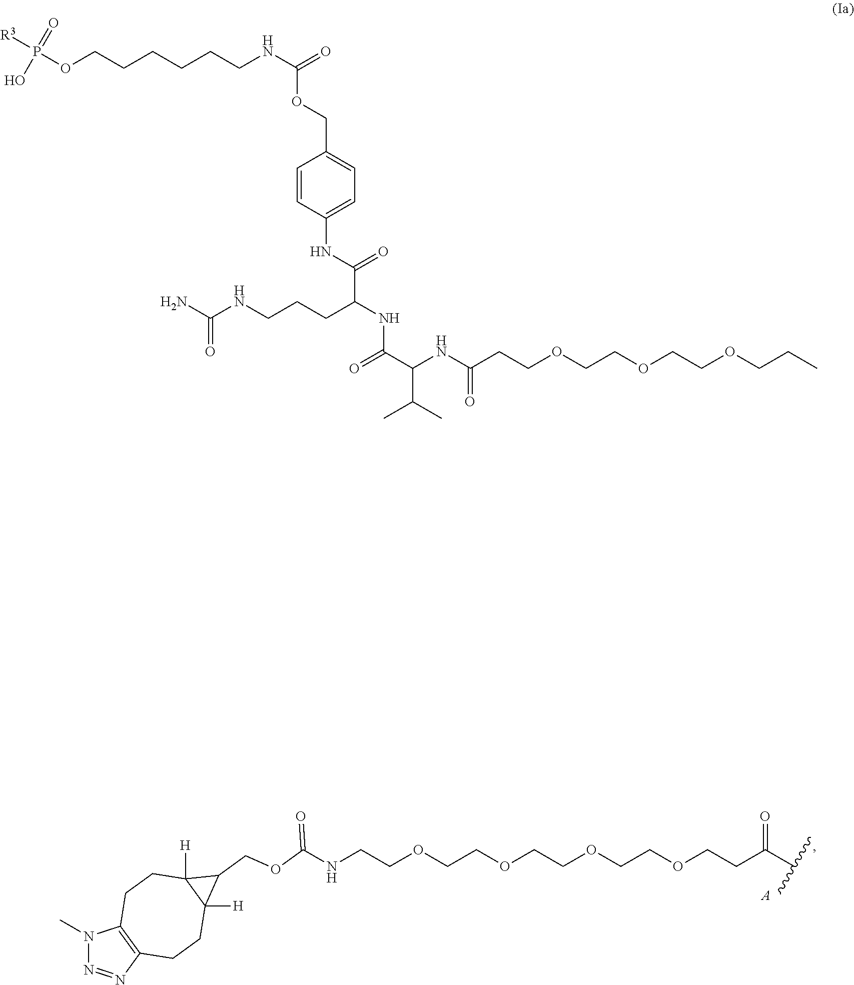

in which R³ comprises an oligonucleotide comprising a nucleobase sequence of CAGCGCCCACCAGUCA (SEQ ID NO: 21) and comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 1 mg to 12 mg of the oligonucleotides of the complexes per kg of the subject, optionally wherein the effective amount provides to the subject 1.8 mg to 10.2 mg or 1.1 mg to 8.2 mg of the oligonucleotides of the complexes per kg of the subject.

56. A method of reducing DMPK expression or treating myotonic dystrophy in a subject, comprising administering intravenously an effective amount of a composition comprising complexes comprising a structure of formula (I): [R¹]ₙ₁—R², wherein:
each R¹ comprises a group of the formula (Ib):

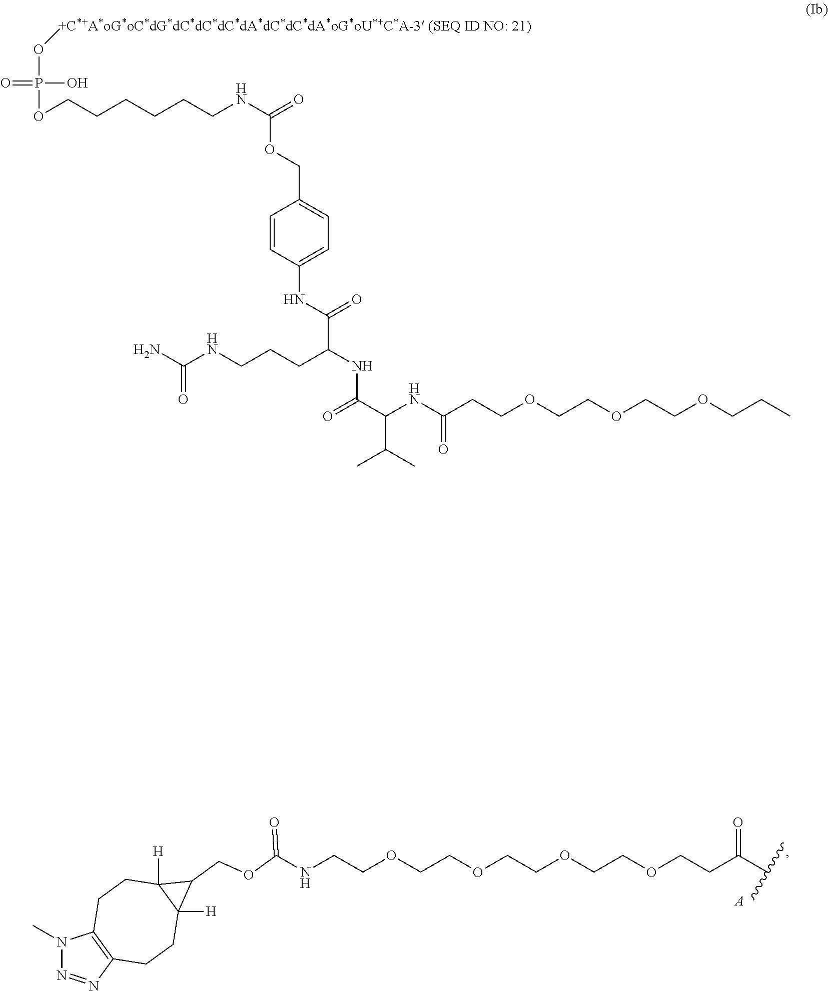

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 1 mg to 12 mg of the oligonucleotides of the complexes per kg of the subject, optionally wherein the effective amount provides to the subject 1.8 mg to 10.2 mg or 1.1 mg to 8.2 mg of the oligonucleotides of the complexes per kg of the subject.

57. A method of reducing DMPK expression or treating myotonic dystrophy in a subject, comprising administering intravenously an effective amount of a composition comprising complexes comprising a structure of formula (I): [R¹]ₙ₁—R², wherein:
each R¹ comprises a group of the formula (Ic):

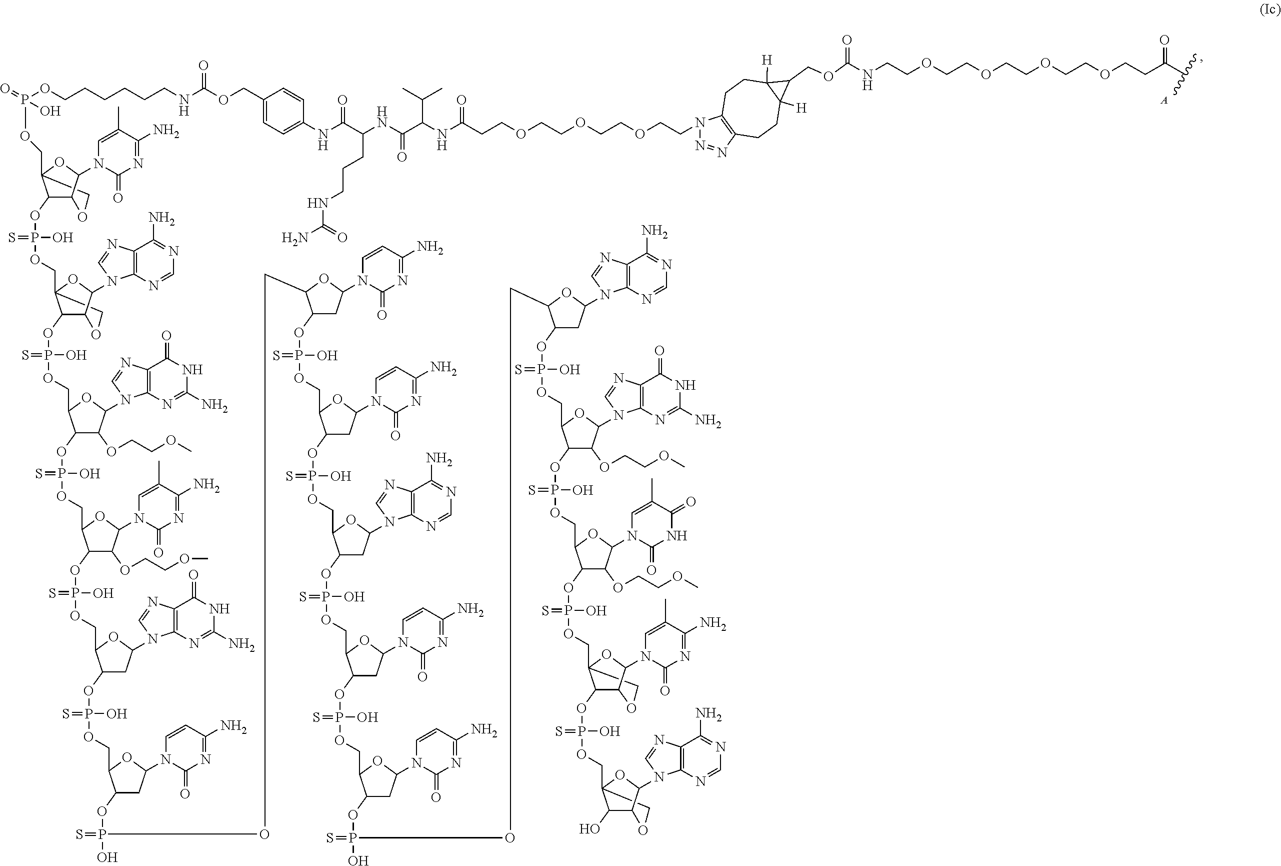

in which the oligonucleotide of R¹ comprises a nucleobase sequence of CAGCGCCCACCAGUCA (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 1 mg to 12 mg of the oligonucleotides of the complexes per kg of the subject, optionally wherein the effective amount provides to the subject 1.8 mg to 10.2 mg or 1.1 mg to 8.2 mg of the oligonucleotides of the complexes per kg of the subject.

58. A method of reducing DMPK expression or treating myotonic dystrophy in a subject, comprising administering intravenously an effective amount of a composition comprising complexes comprising a structure of the formula (Id):

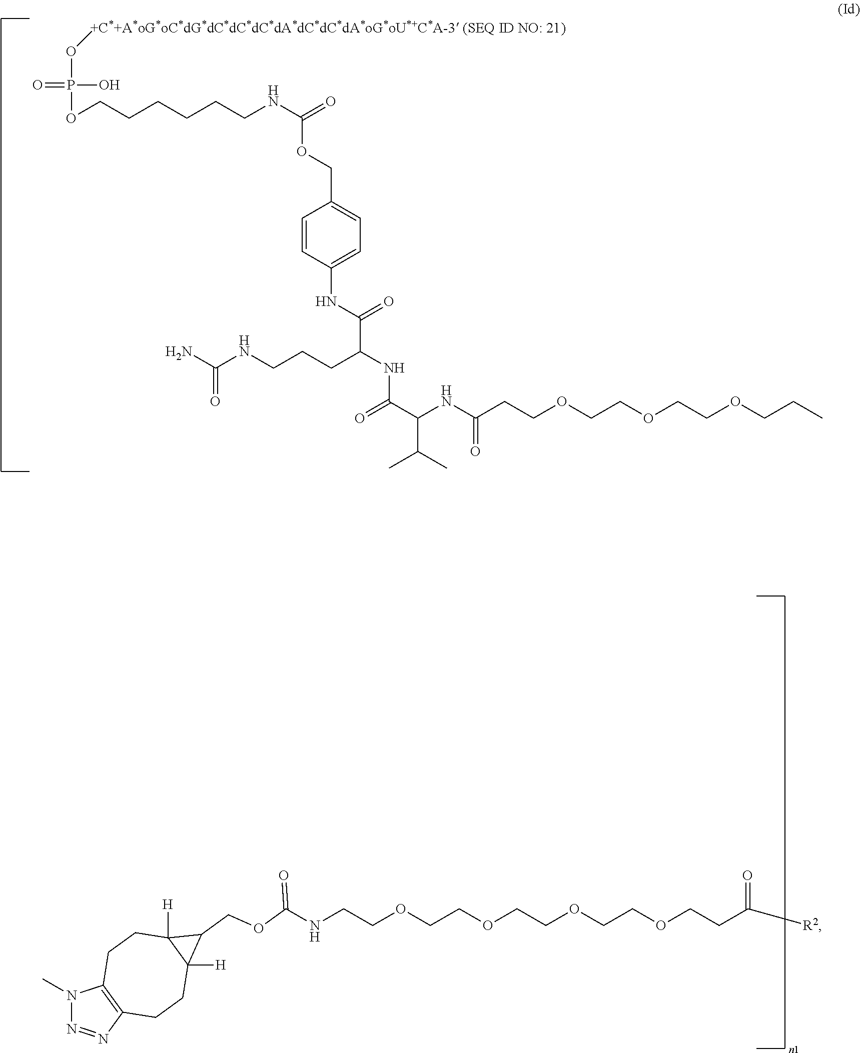

in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5;
wherein the effective amount provides to the subject 1 mg to 12 mg of the oligonucleotides of the complexes per kg of the subject, optionally wherein the effective amount provides to the subject 1.8 mg to 10.2 mg or 1.1 mg to 8.2 mg of the oligonucleotides of the complexes per kg of the subject.

59. The method of any one of embodiments 51-58, wherein the composition is in the form of an aqueous solution and wherein the aqueous solution is at a pH of 7.5 and comprises tris(hydroxymethyl)aminomethane at a concentration of 25 mM, sucrose at a concentration of 10 w/v %, and the complexes at a concentration in the range of 10 mg/mL to 50 mg/mL.

60. The method of any one of embodiments 51-59, wherein the composition is administered once every 4 weeks, once every 8 weeks, or once every 12 weeks for a period of time.

61. The method of any one of embodiments 51-59, wherein the composition is administered once every 4 weeks during a first period of administration, and subsequently administered once every 8 weeks during a second period of administration.

62. The method of any one of embodiments 49-58.1, wherein the subject is human.

63. A method comprising administering to a subject a composition comprising complexes comprising an anti-transferrin receptor 1 (TfR1) antibody covalently linked to one or more oligonucleotides, wherein composition provides to the subject the anti-TfR1 antibody of the complexes in an amount in the range of 5 mg to 110 mg antibody per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, and wherein the oligonucleotides of the complexes comprise the nucleobase sequence of CAGCGCCCACCAGUCA (SEQ ID NO: 21).

64. A method comprising administering to the subject a composition comprising complexes comprising an anti-transferrin receptor 1 (TfR1) antibody covalently linked to one or more oligonucleotides, wherein the composition provides to the subject the oligonucleotides of the complexes in an amount in the range of 1 mg to 12 mg oligonucleotide per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, and wherein the oligonucleotides of the complexes comprise the nucleobase sequence of CAGCGCCCACCAGUCA (SEQ ID NO: 21).

65. The method of any one of embodiments 1-64, wherein the composition further comprises one or more anti-TfR1 antibodies that are not covalently linked to an oligonucleotide.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. Unless otherwise indicated, ranges of values herein are inclusive of their endpoints (e.g., a range of X to Y is inclusive of the values X and Y). It should be understood that recitations herein of a value from X to Y indicates that the specified value falls in the range of X to Y. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A method of reducing DMPK expression and/or treating myotonic dystrophy in a subject, comprising administering to the subject a composition comprising an effective amount of complexes comprising an anti-transferrin receptor 1 (TfR1) antibody covalently linked to one or more oligonucleotides, wherein the effective amount provides to the subject 5 mg to 110 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotides of the complexes comprise the nucleobase sequence of CAGCGCCCACCAGUCA (SEQ ID NO: 21).

2. The method of claim 1, wherein the oligonucleotides of the complexes comprise a 5′-X—Y—Z-3′ configuration, wherein X and Z are flanking regions comprising one or more modified nucleosides and Y is a gap region comprising one or more 2′-deoxyribonucleosides.

3. The method of claim 1 or claim 2, wherein each complex comprises a structure of formula (I): [R¹]ₙ₁—R², wherein:
each R¹ comprises a group of the formula (Ia): 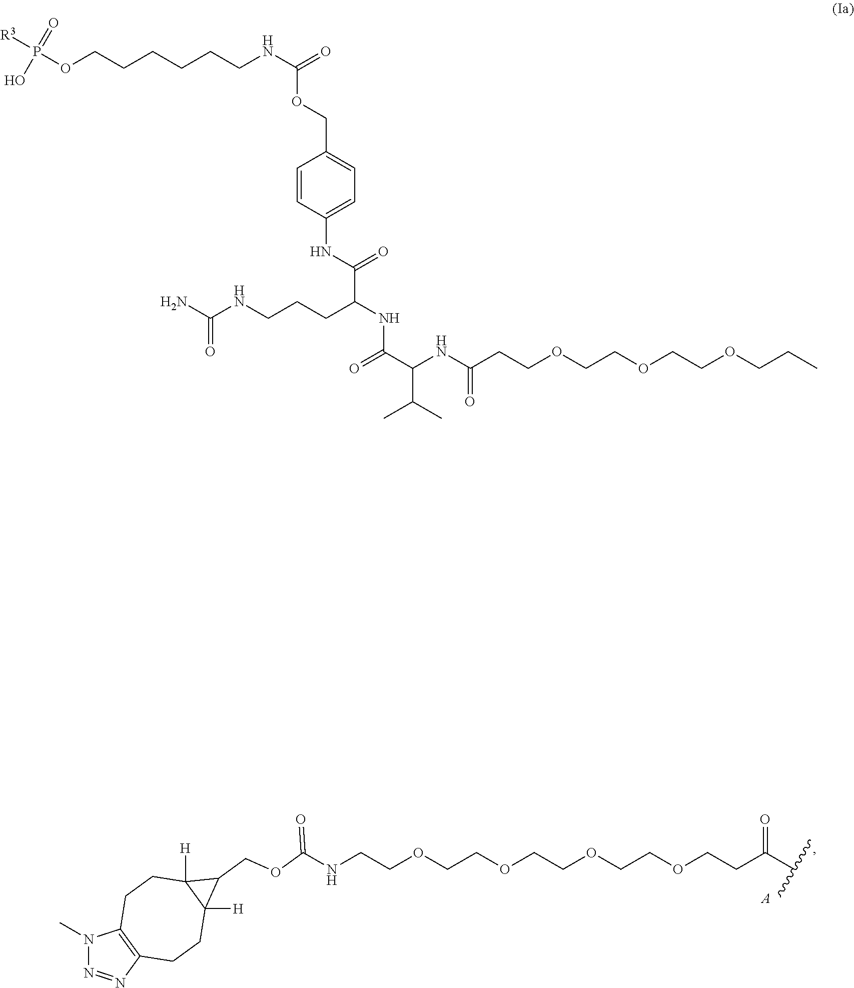
in which R³ comprises an oligonucleotide comprising a nucleobase sequence of CAGCGCCCACCAGUCA (SEQ ID NO: 21) and comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5.

4. The method of claim 1 or claim 2, wherein each complex comprises a structure of formula (I): [R¹]ₙ₁—R², wherein:
each R¹ comprises a group of the formula (Ib): 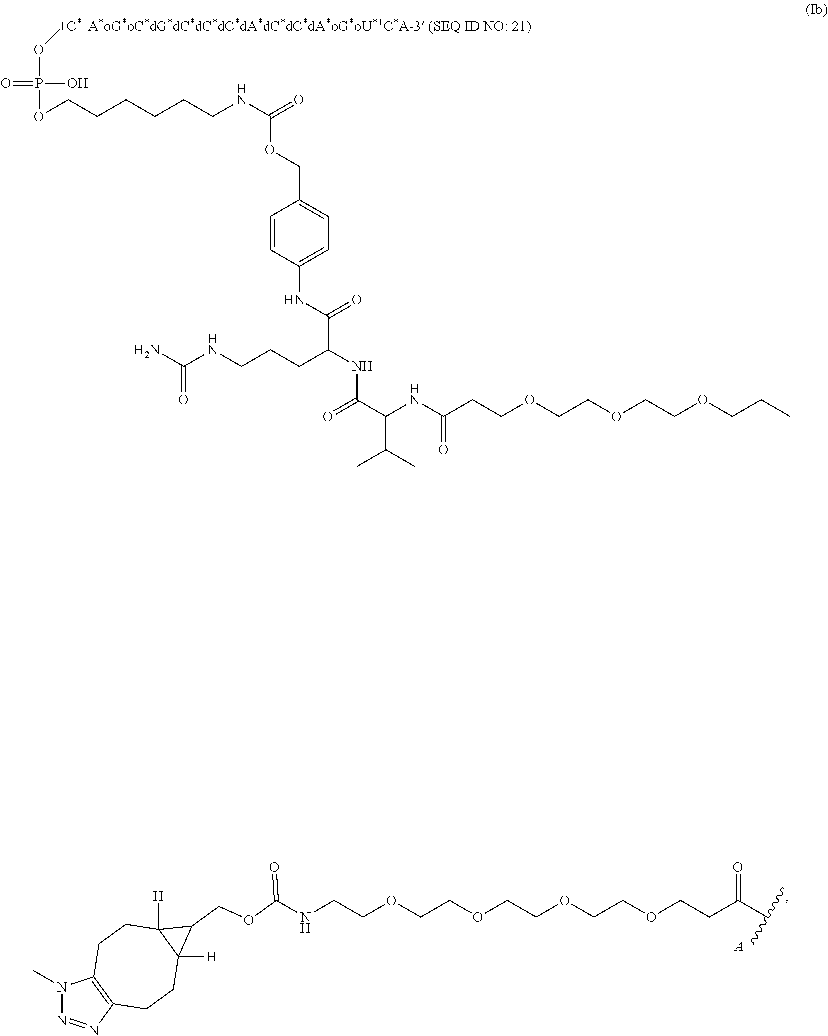
in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5.

5. The method of claim 1 or claim 2, wherein each complex comprises a structure of formula (I): [R¹]ₙ₁—R², wherein:
each R¹ comprises a group of the formula (Ic): 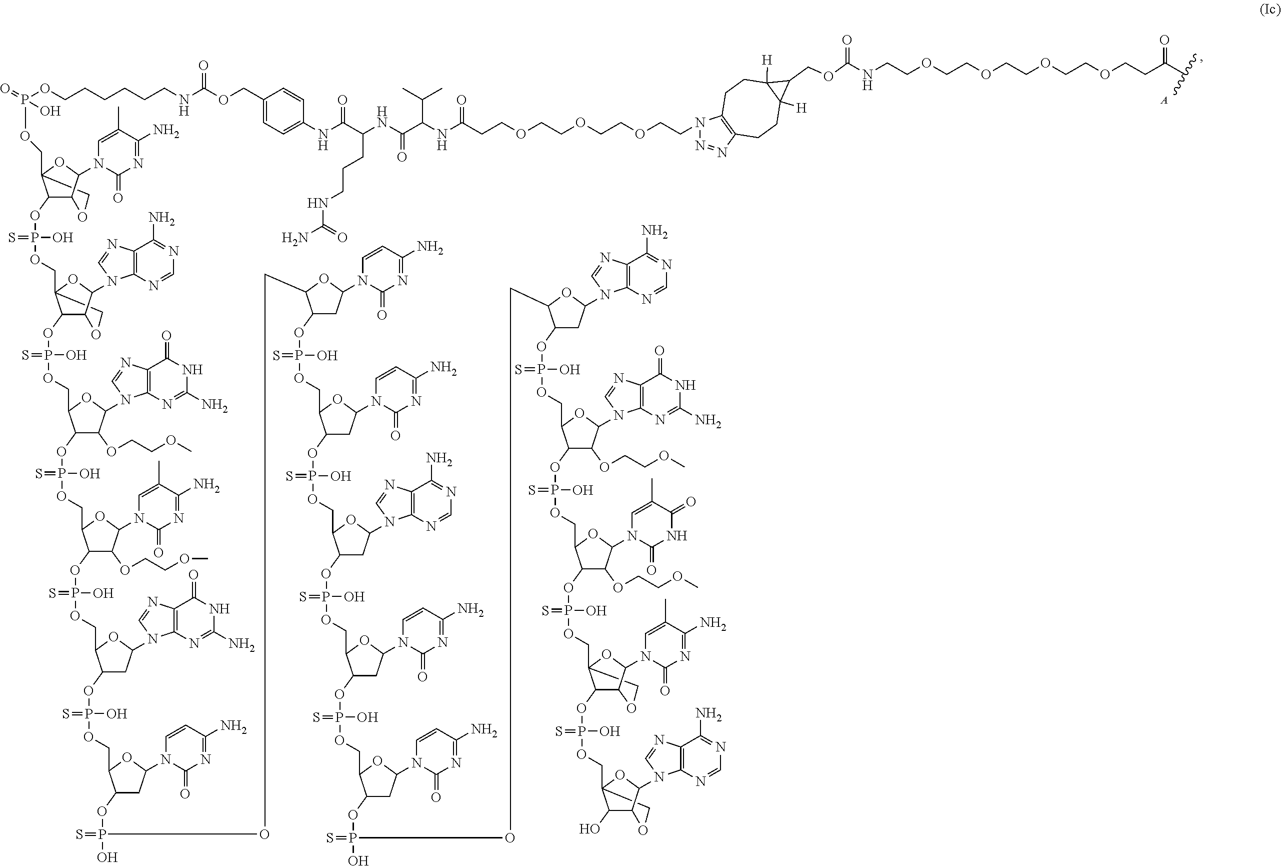
wherein R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5.

6. The method of claim 1 or claim 2, wherein each complex comprises a structure of the formula (Id): 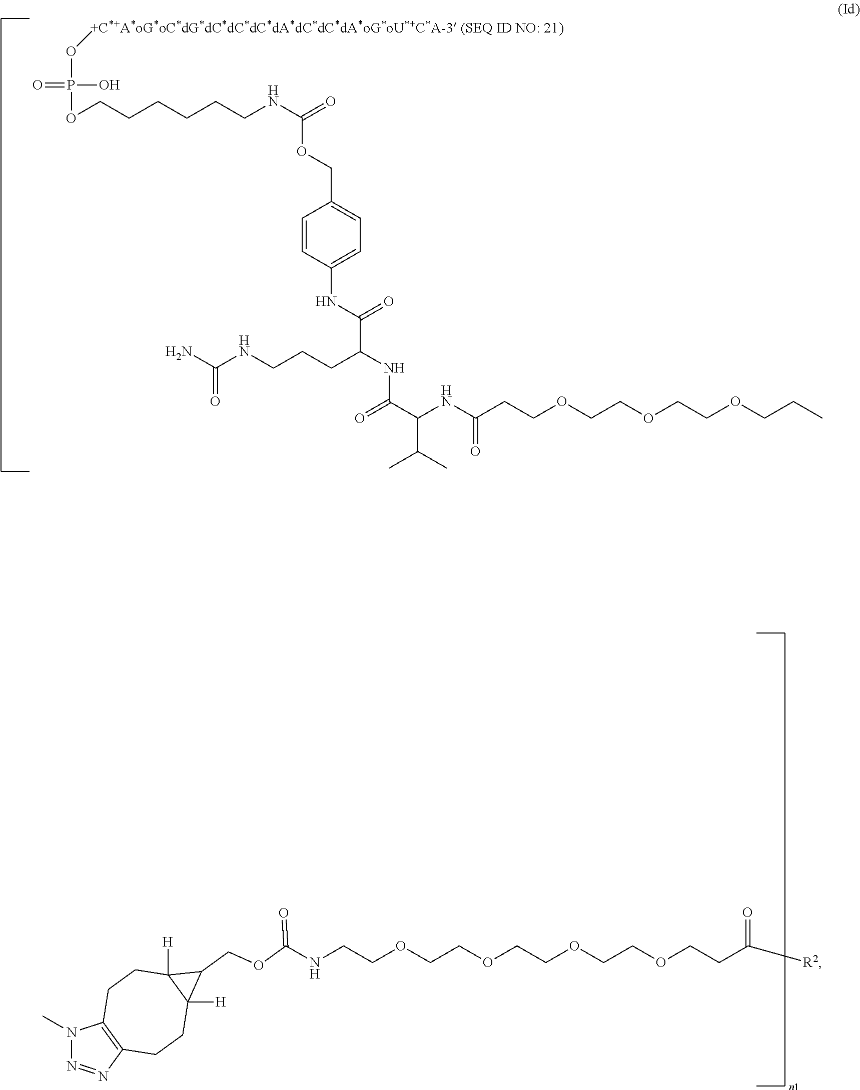
in which +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage, and the oligonucleotide of R¹ comprises a nucleobase sequence of CAGCGCCCACCAGUCA (SEQ ID NO: 21);
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 0.5-5.

7. The method of any one of claims 1-6, wherein the anti-TfR1 antibody is a Fab fragment.

8. The method of any one of claims 1-7, wherein the anti-TfR1 antibody comprises a heavy chain variable region (VH) comprising the amino acid sequence of SEQ ID NO: 17 and a light chain variable region (VL) comprising the amino acid sequence of SEQ ID NO: 18,
optionally wherein the anti-TfR1 antibody comprises a heavy chain comprising the amino acid sequence of SEQ ID NO: 19 and a light chain comprising the amino acid sequence of SEQ ID NO: 20.

9. The method of any one of claims 1-8, wherein the administration occurs one or more times.

10. The method of claim 9, wherein the effective amount of each administration provides to the subject:
(a) 10 mg to 110 mg of the anti-TfR1 antibodies of the complexes per kg of the subject;
(b) 5 mg to 90 mg of the anti-TfR1 antibodies of the complexes per kg of the subject;
(c) 10 mg to 20 mg of the anti-TfR1 antibodies of the complexes per kg of the subject, optionally wherein the effective amount of each administration provides to the subject 13 mg of the anti-TfR1 antibodies of the complexes per kg of the subject;
(d) 18 mg to 36 mg of the anti-TfR1 antibodies of the complexes per kg of the subject, optionally wherein the effective amount of each administration provides to the subject 25 mg of the anti-TfR1 antibodies of the complexes per kg of the subject;
(e) 36 mg to 72 mg of the anti-TfR1 antibodies of the complexes per kg of the subject, optionally wherein the effective amount of each administration provides to the subject 50 mg of the anti-TfR1 antibodies of the complexes per kg of the subject;
(f) 55 mg to 110 mg of the anti-TfR1 antibodies of the complexes per kg of the subject, optionally wherein the effective amount of each administration provides to the subject 75 mg of the anti-TfR1 antibodies of the complexes per kg of the subject;
(g) 6 mg to 12 mg of the anti-TfR1 antibodies of the complexes per kg of the subject, optionally wherein the effective amount of each administration provides to the subject 8 mg of the anti-TfR1 antibodies of the complexes per kg of the subject;
(h) 11 mg to 22 mg of the anti-TfR1 antibodies of the complexes per kg of the subject, optionally wherein the effective amount of each administration provides to the subject 15 mg of the anti-TfR1 antibodies of the complexes per kg of the subject;
(i) 22 mg to 44 mg of the anti-TfR1 antibodies of the complexes per kg of the subject, optionally wherein the effective amount of each administration provides to the subject 30 mg of the anti-TfR1 antibodies of the complexes per kg of the subject; or
(j) 44 mg to 88 mg of the anti-TfR1 antibodies of the complexes per kg of the subject, optionally wherein the effective amount of each administration provides to the subject 60 mg of the anti-TfR1 antibodies of the complexes per kg of the subject.

11. The method of claim 9, wherein the effective amount of each administration provides to the subject:
(a) 20 mg to 43 mg of the anti-TfR1 antibodies of the complexes per kg of the subject, optionally wherein the effective amount of each administration provides to the subject 29 mg of the anti-TfR1 antibodies of the complexes per kg of the subject; or
(b) 26 mg to 53 mg of the anti-TfR1 antibodies of the complexes per kg of the subject, optionally wherein the effective amount of each administration provides to the subject 37 mg of the anti-TfR1 antibodies of the complexes per kg of the subject.

12. The method of any one of claims 1-11, wherein during a period of administration, the composition is administered once every 4 weeks, once every 8 weeks, or once every 12 weeks.

13. The method of claim 12, wherein the period of administration is less than 10 years.

14. The method of claim 12, wherein the period of administration is the remainder of the subject's lifetime.

15. The method of any one of claims 1-11, wherein the composition is administered once every 4 weeks during a first period of administration, and subsequently administered once every 8 weeks during a second period of administration.

16. The method of claim 15, wherein the first period of administration is 8-16 weeks, and/or wherein the second period of administration is 16 weeks to the remainder of the subject's lifetime.

17. The method of any one of claims 1-16, wherein the composition is in the form of an aqueous solution and further comprises tris(hydroxymethyl)aminomethane and sucrose.

18. The method of claim 17, wherein the tris(hydroxymethyl)aminomethane is present in the aqueous solution at a concentration of 25 mM, the sucrose is present in the aqueous solution at a concentration of 10 w/v %, and the aqueous solution is at a pH of 7.5.

19. The method of claim 17 or claim 18, wherein the complexes are present in the composition at a concentration in the range of 10 mg/mL to 50 mg/mL.

20. The method of any one of claims 1-19, wherein the administering reduces DMPK expression in a muscle cell of the subject, optionally wherein reducing DMPK expression comprises reducing the amount of DMPK RNA in the muscle cell, optionally wherein the DMPK RNA amount is reduced in the nucleus of the muscle cell.

21. The method of claim 20, wherein reducing DMPK expression in the muscle cell comprises reducing the amount of DMPK protein in the muscle cell.

22. The method of any one of claims 1-21, wherein the subject is human.

23. The method of any one of claims 1-22, wherein the complex is administered systemically, optionally wherein the complex is administered intravenously, further optionally wherein the complex is administered by infusion.

24. The method of any one of claims 1-23, wherein the composition further comprises one or more anti-TfR1 antibodies that are not covalently linked to an oligonucleotide.
